(19) United States
(12) Patent Application Publication
(10) Pub. No.: US 2025/0312351 A1
(43) Pub. Date: Oct. 9, 2025

(54) COMBINATION THERAPIES USING PRMT5 INHIBITORS AND SOS1 INHIBITORS FOR THE TREATMENT OF CANCER

(71) Applicant: MIRATI THERAPEUTICS, INC., PRINCETON, NJ (US)

(72) Inventors: LARS DANIEL ENGSTROM, ESCONDIDO, CA (US); PETER OLSON, SAN DIEGO, CA (US); JILL HALLIN, SAN DIEGO, CA (US)

(73) Assignee: MIRATI THERAPEUTICS, INC., PRINCETON, NJ (US)

(21) Appl. No.: 19/171,416

(22) Filed: Apr. 7, 2025

Related U.S. Application Data
(60) Provisional application No. 63/631,029, filed on Apr. 8, 2024.

(51) Int. Cl. A61K 31/5377; A61K 31/502

(52) U.S. Cl. CPC A61K 31/5377; A61K 31/502

(57) ABSTRACT

This disclosure relates to methods of treating cancer. This disclosure further relates to treating cancer in a subject with compounds that are methylthioadenosine (MTA)-cooperative PRMT5 inhibitors, particularly in combination with SOS1 checkpoint inhibitors.

### CROSS REFERENCE TO RELATED APPLICATIONS

This application claims the benefit of U.S. Provisional Application No. 63/631,029, filed Apr. 8, 2024, the entire content of which is hereby incorporated herein by reference.

### BACKGROUND OF THE DISCLOSURE

### Field of the Disclosure

This disclosure relates to methods of treating cancer. This disclosure further relates to treating cancer in a subject with compounds that are inhibitors of protein arginine N-methyl transferase 5 (PRMT5), particularly in combination with SOS1 inhibitors.

### Description of Related Art

PRMT5 is a type II arginine methyltransferase that catalyzes the transfer of a methyl group from S-adenosyl-L-methionine (SAM) to an omega-nitrogen of the guanidino function of protein L-arginine residues (omega-monomethylation) and the transfer of a second methyl group to the other omega-nitrogen, yielding symmetric dimethylarginine (sDMA). PRMT5 forms a complex with methylosome protein 50 (MEP50), which is required for substrate recognition and orientation and is also required for PRMT5-catalyzed histone 2A and histone 4 methyltransferase activity (e.g., see Ho et al. (2013) PLoS ONE 8(2): e57008).

Homozygous deletions of p16/CDKN2a are prevalent in cancer and these mutations commonly involve the co-deletion of adjacent genes, including the gene encoding methylthioadenosine phosphorylase (MTAP). It is estimated that approximately 15% of all human cancers have a homozygous deletion of the MTAP gene (e.g., see Firestone & Schramm (2017) J. Am. Chem Soc. 139(39):13754-13760).

Cells lacking MTAP activity have elevated levels of the MTAP substrate, methylthioadenosine (MTA), which is a potent inhibitor of PRMT5. Inhibition of PRMT5 activity results in reduced methylation activity and increased sensitivity of cellular proliferation to PRMT5 depletion or loss of activity. Hence, the loss of MTAP activity reduces methylation activity of PRMT5 making the cells selectively dependent on PRMT5 activity.

The Ras family comprises v-Ki-ras2 Kirsten rat sarcoma viral oncogene homolog (KRAS), neuroblastoma RAS viral oncogene homolog (NRAS), and Harvey murine sarcoma virus oncogene (HRAS) and critically regulates cellular division, growth and function in normal and altered states including cancer (see e.g., Simanshu et al. Cell, 2017. 170(1): p. 17-33; Matikas et al., Crit Rev Oncol Hematol, 2017. 110: p. 1-12). RAS proteins are activated by upstream signals, including receptor tyrosine kinases (RTKs), and transduce signals to several downstream signaling pathways such as the mitogen-activated protein kinase (MAPK)/extracellular signal-regulated kinases (ERK) pathway. Hyperactivation of RAS signaling is frequently observed in cancer as a result of mutations or alterations in RAS genes or other genes in the RAS pathway. The identification of strategies to inhibit RAS and RAS signaling are predicted to be useful for the treatment of cancer and RAS-regulated disease states.

RAS proteins are guanosine triphosphatases (GTPases) that cycle between an inactive, guanosine diphosphate (GDP)-bound state and an active guanosine triphosphate (GTP)-bound state. Son of sevenless homolog 1 (SOS1) is a guanine nucleotide exchange factor (GEF) that mediates the exchange of GDP for GTP, thereby activating RAS proteins. RAS proteins hydrolyze GTP to GDP through their intrinsic GTPase activity which is greatly enhanced by GTPase-activating proteins (GAPs). This regulation through GAPs and GEFs is the mechanism whereby activation and deactivation are tightly regulated under normal conditions. Mutations at several residues in all three RAS proteins are frequently observed in cancer and result in RAS remaining predominantly in the activated state (Sanchez-Vega et al., Cell, 2018. 173: p. 321-337 Li et al., Nature Reviews Cancer, 2018. 18: p. 767-777). Mutations at codon 12 and 13 are the most frequently mutated RAS residues and prevent GAP-stimulated GTP hydrolysis by blocking the interaction of GAP proteins and RAS. Recent biochemical analyses however, demonstrated these mutated proteins still require nucleotide cycling for activation based on their intrinsic GTPase activity and/or partial sensitivity to extrinsic GTPases. As such, mutant RAS proteins are sensitive to inhibition of upstream factors such as SOS1 or SHP2, another upstream signaling molecule required for RAS activation (Hillig, 2019; Patricelli, 2016; Lito, 2016; Nichols, 2018).

The three main RAS-GEF families that have been identified in mammalian cells are SOS, RAS-GRF and RAS-GRP (Rojas, 2011). RAS-GRF and RAS-GRP are expressed in the cells of the central nervous system and hematopoietic cells, respectively, while the SOS family is ubiquitously expressed and is responsible for transducing RTK signaling. The SOS family comprises SOS1 and SOS2 and these proteins share approximately 70% sequence identity. SOS1 appears to be much more active than SOS2 due to the rapid degradation of SOS2. The mouse SOS2 knockout is viable whereas the SOS1 knockout is embryonic lethal. A tamoxifen-inducible SOS1 knockout mouse model was used to interrogate the role of SOS1 and SOS2 in adult mice and demonstrated the SOS1 knockout was viable but the SOS1/2 double knockout was not viable (Baltanas, 2013) suggesting functional redundancy and that selective inhibition of SOS1 may have a sufficient therapeutic index for the treatment of SOS1—RAS activated diseases.

SOS proteins are recruited to phosphorylated RTKs through an interaction with growth factor receptor bound protein 2 (GRB2). Recruitment to the plasma membrane places SOS in close proximity to RAS and enables SOS-mediated RAS activation. SOS proteins bind to RAS through a binding site that promotes nucleotide exchange as well as through an allosteric site that binds GTP-bound RAS-family proteins and increases the function of SOS (Freedman et al., Proc. Natl. Acad. Sci, USA 2006. 103(45): p. 16692-97). Binding to the allosteric site relieves steric occlusion of the RAS substrate binding site and is therefore required for nucleotide exchange. Retention of the active conformation at the catalytic site following interaction with the allosteric site is maintained in isolation due to strengthened interactions of key domains in the activated state. SOS1 mutations are found in Noonan syndrome and several cancers including lung adenocarcinoma, embryonal rhabdomyosarcoma, Sertoli cell testis tumor and granular cell tumors of the skin (see e.g., Denayer, E., et al, Genes Chromosomes Cancer, 2010. 49(3): p. 242-52).

GTPase-activating proteins (GAPs) are proteins that stimulate the low intrinsic GTPase activity of RAS family members and therefore converts active GTP-bound RAS proteins into inactive, GDP-bound RAS proteins (e.g., see Simanshu, D. K., Cell, 2017, Ras Proteins and their Regulators in Human Disease). While activating alterations in the GEF SOS1 occur in cancers, inactivating mutations and loss-of-function alterations in the GAPs neurofibromin 1 (NF-1) or neurofibromin 2 (NF-2) also occur creating a state where SOS1 activity is unopposed and activity downstream of the pathway through RAS proteins is elevated.

For all the foregoing reasons, there is a need to develop combination therapies using PRMT5 inhibitors and SOS1 inhibitors to treat a wide range of cancers.

### SUMMARY OF THE DISCLOSURE

One aspect of the disclosure provides methods for treating cancer in a subject. Such methods include administering to the subject a therapeutically effective amount of SOS1 inhibitor and a therapeutically effective amount of a PRMT5 inhibitor.

Also provided herein is a method for treating cancer in a subject in need thereof. Such methods may include determining that the cancer is associated with MTAP homozygous deletion (e.g., an MTAP-associated cancer). Such methods further include administering to the subject a therapeutically effective amount of a SOS1 inhibitor and a therapeutically effective amount of a PRMT5 inhibitor.

These and other features and advantages of the present invention will be more fully understood from the following detailed description taken together with the accompanying claims. It is noted that the scope of the claims is defined by the recitations therein and not by the specific discussion of features and advantages set forth in the present description.

### DETAILED DESCRIPTION OF THE DISCLOSURE

Before the disclosed processes and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

As describe above, both MTAP^{del} and NF1 gene mutations are prevalent in many cancers. The present inventors have advantageously found a combination therapy to target cancers with both of these characteristics. In one aspect, the present disclosure provides a method for treating cancer in a subject, the method includes administering to the subject a therapeutically effective amount of a SOS1 inhibitor and a therapeutically effective amount of a protein arginine N-methyl transferase 5 (PRMT5) inhibitor, wherein the PRMT5 inhibitor is methylthioadenosine (MTA)-cooperative PRMT5 inhibitor.

### Combination Therapy

In view of the present disclosure, the methods and compositions described herein can be configured by the person of ordinary skill in the art to meet the desired need. The present disclosure provides improvements in treating cancer in a subject. As used herein, the terms “subject” or “patient” are used interchangeably, refers to any animal, including mammals, and most preferably humans.

The methods provided herein may be used for the treatment of a wide variety of cancer including tumors such as lung, prostate, breast, brain, skin, cervical carcinomas, testicular carcinomas, etc. More particularly, cancers that may be treated by the compositions and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

In certain embodiments of the methods of the disclosure, the cancer is a MTAP-associated cancer. For example, in certain embodiments, the cancer comprises MTAP gene homozygous deletion (MTAP^{DEL}). The subject may be identified or diagnosed as having MTAP-associated cancer where, for example, MTAPD^{EL} is determined using a suitable assay or a kit. Alternatively, the subject is suspected of having MTAP-associated cancer or the subject has a clinical record indicating that the subject has MTAP-associated cancer.

In certain embodiments of the methods of the disclosure, the cancer is a NF1 gene mutation cancer. For example, in certain embodiments, the cancer comprises a NF1 gene mutation. The subject may be identified or diagnosed as having NF1 cancer where, for example, NF1 gene mutation is determined using a suitable assay or a kit. Alternatively, the subject is suspected of having NF1 gene-associated cancer or the subject has a clinical record indicating that the subject has NF1-associated cancer.

In some embodiments of any of the methods or uses described herein, an assay is used to determine whether the patient has MTAP^{DEL} and/of NF1 gene mutation using a sample (e.g., a biological sample or a biopsy sample such as a paraffin-embedded biopsy sample) from a subject. Such assay includes, but is not limited to, next generation sequencing, immunohistochemistry, fluorescence microscopy, break apart FISFI analysis, Southern blotting. Western blotting, FACS analysis, Northern blotting, and PCR-based amplification (e.g., RT-PCR and quantitative real-time RT-PCR). As is well known in the art, the assays are typically performed, e.g., with at least one labelled nucleic acid probe or at least one labelled antibody or antigen-binding fragment thereof.

In certain embodiments, the cancer in the methods of the disclosure is selected from lung cancer, pancreatic cancer, colon cancer, head and neck cancer, bladder cancer, esophageal cancer, lymphoma, stomach cancer, skin cancer, breast cancer, and brain cancer.

In certain embodiments, the cancer in the methods of the disclosure is a malignant peripheral nerve sheath tumor (MPNST). Malignant peripheral nerve sheath tumors are a form of cancer of the connective tissue or sheath that surrounds and protects peripheral nerves. Malignant peripheral nerve sheath tumors were previously named neurofibrosarcomas. Malignant peripheral nerve sheath tumors grow in any of the soft tissues of the body, such as muscle, fat, tendons, ligaments, lymph and blood vessels, nerves, and other tissue that connects and supports the body. MPNST grows quickly and can spread to other parts of the body.

The PRMT5 inhibitor of the disclosure and/or the SOS1 inhibitor of the disclosure may be provided as a pharmaceutical composition comprising a therapeutically effective amount of such inhibitor and a pharmaceutically acceptable carrier, excipient, and/or diluents. The PRMT5 inhibitor of the disclosure and/or the SOS1 inhibitor of the disclosure may be formulated by any method well known in the art and may be prepared for administration by any route, including, without limitation, parenteral, oral, sublingual, transdermal, topical, intranasal, intratracheal, or intrarectal. In certain embodiments, the PRMT5 inhibitor of the disclosure and/or the SOS1 inhibitor of the disclosure are administered intravenously in a hospital setting. In certain other embodiments, administration may preferably be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, pharmaceutical compositions of the disclosure may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18^{th} Edition, ed. A. Gennaro, Mack Publishing Co., Easton, Pa., 1990.

The PRMT5 inhibitor and the SOS1 inhibitor of the disclosure are administered in a therapeutically effective amount. As used herein, the phrase “therapeutically effective amount” or “effective amount” refers to the amount of active agent that elicits the biological or medicinal response that is being sought in a tissue, system, subject or human by a researcher, medical doctor or other clinician. In general, the therapeutically effective amount is sufficient to deliver the biological or medicinal response to the subject without causing serious toxic effects. A dose of the active agent may be in the range from about 0.01 to 300 mg/kg per day, such as 0.1 to 100 mg/kg per day, more generally 0.5 to about 25 mg/kg body weight of the recipient per day. A typical topical dosage will range from 0.01 to 3% wt/wt in a suitable carrier.

In certain embodiments of the methods of the disclosure, the therapeutically effective amount of the PRMT5 inhibitor is in the range of about 0.01 to 300 mg/kg per day. For example, in certain embodiments, the therapeutically effective amount of the PRMT5 inhibitor is in the range of about 0.1 to 100 mg/kg per day, or 25 to 100 mg/kg per day, or 50 to 100 mg/kg per day.

In certain embodiments, the therapeutically effective amount of the PRMT5 inhibitor is less than 1% of, e.g., less than 10%, or less than 25%, or less than 50% of the clinically-established therapeutic amount (e.g., such as the amount required when the PRMT5 inhibitor is administered by itself).

In certain embodiments, the therapeutically effective amount of the PRMT5 inhibitor is administered once daily.

In certain embodiments of the methods of the disclosure, the therapeutically effective amount of the SOS1 inhibitor is in the range of about 0.01 to 300 mg/kg per day. For example, in certain embodiments, the therapeutically effective amount of the SOS1 inhibitor is in the range of about 0.1 to 100 mg/kg per day, or 0.1 to 50 mg/kg per day, or 10 to 100 mg/kg per day, or 10 to 50 mg/kg per day.

In certain embodiments, the therapeutically effective amount of the SOS1 inhibitor is less than 1% of, e.g., less than 10%, or less than 25%, or less than 50% of the clinically-established therapeutic amount (e.g., such as the amount required when the SOS1 inhibitor is administered by itself).

Combination therapy, in defining use of PRMT5 inhibitor and the SOS1 inhibitor of the present disclosure, is intended to embrace administration of each agent in a sequential manner in a regimen that will provide beneficial effects of the drug combination (e.g., the PRMT5 inhibitor and the SOS1 inhibitor of the disclosure can be formulated as separate compositions that are given sequentially), and is intended as well to embrace co-administration of these agents in a substantially simultaneous manner, such as in a single dosage form having a fixed ratio of these active agents or in multiple or a separate dosage forms for each agent. The disclosure is not limited in the sequence of administration: the PRMT5 inhibitor of the disclosure may be administered either prior to or after (i.e., sequentially), or at the same time (i.e., simultaneously) as administration of the SOS1 inhibitor of the disclosure.

The methods of disclosure are useful as a first-line treatment. Thus, in certain embodiments of the methods of the disclosure, the subject has not previously received another first-line of therapy.

The methods of disclosure are also useful as a first-line maintenance or a second-line or later treatment. Thus, in certain embodiments of the methods of the disclosure, the subject has previously completed another first-line of therapy. For example, the methods of the disclosure, in certain embodiments, may provide a delay in progression and relapse of cancer in subjects that have previously completed another first-line chemotherapy. For example, in certain embodiments, the subject has previously completed a platinum- and/or taxane-based chemotherapy (e.g., carboplatin, cisplatin, oxaliplatin, paclitaxel, docetaxel, and the like). In certain embodiments of the methods of the disclosure, the subject has previously completed another first-line chemotherapy and is in partial response to such chemotherapy.

### SOS1 Inhibitors

As described above, the methods of the disclosure include administering a SOS1 inhibitor.

As used herein, “SOS1” refers to a mammalian Son of sevenless homolog 1 (SOS1) enzyme.

A “SOS1-associated disease or disorder” as used herein refers to diseases or disorders associated with or mediated by or having an activating SOS1 mutation. Examples of activating SOS1 mutations include SOS1 N233S and SOS1 N233Y mutations.

As used herein, “SOS1 N233S” refers to a mutant form of a mammalian SOS1 protein that contains an amino acid substitution of a serine for a glutamine at amino acid position 233. The assignment of amino acid codon and residue positions for human SOS1 is based on the amino acid sequence identified by UniProtKB/Swiss-Prot Q07889: Variant p.Gln233Ser.

As used herein, “SOS1 N233Y” refers to a mutant form of a mammalian SOS1 protein that contains an amino acid substitution of a tyrosine for a glutamine at amino acid position 233. The assignment of amino acid codon and residue positions for human SOS1 is based on the amino acid sequence identified by UniProtKB/Swiss-Prot Q07889: Variant p.Gln233Tyr.

As used herein, an “SOS1 inhibitor” refers to compounds of the present invention that are represented by Formula (I) as described herein. These compounds are capable of negatively inhibiting all or a portion of the interaction of SOS1 with Ras family mutant or SOS1 activating mutation thereby reducing and/or modulating the nucleotide exchange activity of Ras family member—SOS1 complex.

As used herein, a “NF-1/NF-2-associated disease or disorder” refers to diseases or disorders associated with or mediated by or having a loss-of-function mutation in the neurofibromin (NF-1) gene or neurofibromin 2 (NF-2) gene.

In various embodiments of the methods of the disclosure, the SOS1 inhibitor is selected from BI 1701963, BTX-B01, RGT-018, HM99462, RMC-5845, and combinations thereof.

A “SOS1 inhibitor” as used herein refers to compounds of the disclosure as described herein. These compounds are capable of negatively modulating or inhibiting all or a portion of the enzymatic activity of the SOS1.

In certain embodiments, the SOS1 inhibitor of the disclosure is any one of the SOS1 inhibitors disclosed in International patent publication No. WO 2021/1127429, published 24 Jun. 2021, incorporated by reference in its entirety.

In certain embodiments, the SOS1 inhibitor of the disclosure is any one of the SOS1 inhibitors disclosed in International patent publication No. WO2021/173524, published 2 Sep. 2021, incorporated by reference in its entirety.

In certain embodiments, the SOS1 inhibitor of the disclosure is any one of the SOS1 inhibitors disclosed in International patent publication No. WO2022/26465, published 3 Feb. 2022, incorporated by reference in its entirety.

In certain embodiments, the SOS1 inhibitor of the disclosure is any one of the SOS1 inhibitors disclosed in International patent publication No. WO2023/059597, published 13 Apr. 2023, incorporated by reference in its entirety.

In other embodiments of the methods of the disclosure, the SOS1 inhibitor is a compound of

### Formula 1:

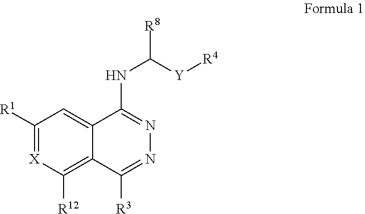

or a pharmaceutically acceptable salt thereof,
wherein:
R¹ is hydrogen, hydroxyl, C1-C6 alkyl, alkoxy, —N(R⁶)₂, —NR⁶C(O)R⁶, —C(O)N(R⁶)₂, —SO₂alkyl, —SO₂NR⁶alkyl, cycloalkyl, -Q-heterocyclyl, aryl, or heteroaryl, wherein the cycloalkyl, the heterocyclyl, the aryl, and the heteroaryl are each optionally substituted with one or more R² or L-R²;
each Q is independently a bond, O or NR⁶;
X is N or CR⁷;
each R² is independently C1-C3 alkyl, oxo (i.e., (C═O), hydroxy, halogen, cyano, hydroxyalkyl, haloalkyl, alkoxy, —C(O)N(R⁶)₂, —N(R⁶)₂, —SO₂alkyl, —NR⁶C(O)C1-C3 alkyl, —C(O)cycloalkyl, —C(O)C1-C3 alkyl, —C(O)heterocyclyl, aryl, heteroaryl or heterocyclyl, wherein the cycloalkyl, the heterocyclyl, the aryl, the heteroaryl or the heterocyclyl are each optionally substituted with one or more R¹¹;
R³ is hydrogen, C1-C6 alkyl, alkoxy, —N(R¹⁰)₂, -L-N(R¹⁰)₂, cycloalkyl, haloalkyl or heterocyclyl, wherein the C1-C6 alkyl, the cycloalkyl and the heterocyclyl are each optionally substituted with one or more R⁹;
Y is a bond or heteroarylene;
R⁴ is aryl or heteroaryl, each optionally substituted with one or more R⁵;
each R⁵ is independently hydroxy, halogen, cyano, hydroxyalkyl, alkoxy, C1-C3 alkyl, haloalkyl, haloalkyl-OH, —N(R⁶)₂, -L-N(R⁶)₂ or —SO₂alkyl;
L is C1-C3 alkylene;
each R⁶ is independently hydrogen, C1-C3 alkyl, haloalkyl, or cycloalkyl;
R⁷ is hydrogen, cyano, or alkoxy;
R⁸ is C₁-C2 alkyl or haloC1-C2 alkyl;
each R⁹ is independently hydroxy, halogen, amino, cyano, alkoxy, or C1-C3 alkyl;
each R¹⁰ is independently hydrogen, C1-C3 alkyl or cycloalkyl;
each R^{II} is independently C1-C3 alkyl, halogen or haloalkyl; and
R¹² is hydrogen, halogen or C1-C3 alkyl.

In one embodiment for compounds of Formula (I), X is N. In certain embodiments wherein X is N, R¹ is alkoxy. In one embodiment, the alkoxy is methoxy.

In one embodiment for compounds of Formula (I), X is N. In certain embodiments wherein X is N, R¹ is -Q-heterocyclyl optionally substituted with one or more R². In certain embodiments, R¹ is -Q-heterocyclyl, and wherein Q is a bond and the heterocyclyl is morpholinyl, piperazinyl, or piperazinone optionally substituted with one or more R². In certain embodiments, the heterocyclyl is morpholinyl or piperazinyl, Y is a bond, and R⁴ is aryl optionally substituted with one or more R⁵. In one embodiment, the heterocyclyl is morpholinyl, piperazinyl, or piperazinone, Y is heteroarylene, and R⁴ is aryl optionally substituted with one or more R⁵.

In certain embodiments of the invention, R¹ is -Q-heterocyclyl, and wherein the heterocyclyl is bridged morpholinyl, bridged piperazinyl, or bridged piperazinone.

In certain embodiments of the invention, R¹ is -Q-heterocyclyl, and wherein the heterocyclyl is spirocyclic ring system containing two or more rings. In certain of these embodiments, the spirocyclic ring system comprises two rings each containing a heteroatom. In certain other of these embodiments, the spirocyclic ring system contains a ring with no heteroatom (i.e., one ring with a heteroatom, and one ring without a heteroatom).

In certain embodiments of the invention, R¹ is heteroaryl, wherein the heterocyclyl is optionally substituted with one or more R² or L-R². In certain of these embodiments, the heteroaryl is a bicyclic or tricyclic ring system comprising, in additional to one or more aromatic ring, a non-aromatic ring, for example a bicyclic or tricyclic ring system such as 5,6,7,8-tetrahydro-[1,2,4]triazolopyrazinyl, 5,6,7,8-tetrahydroimidazopyrazinyl, 2,4,5,6-tetrahydropyrrolopyrazolyl, 1,2,3,4-tetrahydrobenzo[4,5]imidazopyrazinyl or 4,5,6,7-tetrahydropyrazolopyrazinyl.

In one embodiment for compounds of Formula (I), X is CR⁷. In one embodiment when X is CR⁷, R⁷ is cyano.

In one embodiment for compounds of Formula (I), X is CR⁷. In one embodiment when X is CR⁷, R⁷ is hydrogen.

In one embodiment for compounds of Formula (I), X is CR⁷, R⁷ is hydrogen, R¹ is hydrogen.

In another embodiment, R¹ is hydroxyl. In certain embodiments, R¹ is —N(R⁶)₂. In one embodiment, wherein R¹ is —N(R⁶)₂ and each R⁶ is C1-C3 alkyl. In one embodiment, each C1-C3 alkyl group is methyl. In other embodiments R¹ is —NR⁶C(O)R⁶. In one embodiment, each C1-C3 alkyl is methyl. In one embodiment, the R⁶ of the NR⁶ is hydrogen and R⁶ of the C(O)R⁶ is C1-C3 alkyl.

In another embodiment when X is CR⁷ and R⁷ is hydrogen, R¹ is —C(O)N(R⁶)₂. In one embodiment, each C1-C3 alkyl is methyl. In one embodiment, each C1-C3 alkyl is hydrogen. In certain embodiments, R¹ is —SO₂alkyl or —SO₂NR⁶alkyl. In one embodiment, R1 is —SO₂NR⁶alkyl and R⁶ is hydrogen. In other embodiments, R¹ is cycloalkyl optionally substituted with one or more R². In one embodiment, the cycloalkyl is cyclobutyl, cyclopentyl or cyclohexyl, each optionally substituted with one or more R². In one embodiment, the cyclobutyl, cyclopentyl or the cyclohexyl are substituted with one R², wherein R² is C1-C3 alkyl, alkoxy, hydroxyl or —N(R⁶)₂. In one embodiment, R² is —N(R⁶)₂ and each R⁶ is C1-C3 alkyl. In one embodiment, each C1-C3 alkyl is methyl.

In another embodiment when X is CR⁷ and R⁷ is hydrogen, R¹ is -Q-heterocyclyl optionally substituted with one or more R². In one embodiment, Q is a bond and the heterocyclyl is morpholinyl, piperdinyl, piperazinyl, N-methyl piperazinyl, piperazinone, 1-methyl-piperazin-2-one, diazepanyl, 6,6-difluoro-1,4-diazepan-1-yl or 4-methylthiomorpholine 1,1-dioxide. In another embodiment, Q is a bond and the heterocyclyl is pyrrolidinyl or tetrahydropyranyl, each optionally substituted with one or more R². In one embodiment, the pyrrolidinyl or the tetrahydropyranyl are substituted with one R², wherein R² is C1-C3 alkyl, alkoxy, hydroxyl or —N(R⁶)₂.

In another embodiment when X is CR⁷ and R⁷ is hydrogen, R¹ is -Q-heterocyclyl, Q is a bond and the heterocyclyl is piperazinyl substituted with one R², wherein R² is heteroaryl optionally substituted with one or more R¹¹. In one embodiment, the heteroaryl is pyrazolyl substituted with two R¹¹, wherein each R¹¹ is C1-C3 alkyl.

In another embodiment when X is CR⁷ and R⁷ is hydrogen, R¹ is -Q-heterocyclyl, Q is a bond and the heterocyclyl is piperazinyl substituted with one R², wherein R² is —C(O)cycloalkyl or —C(O)heterocyclyl, wherein the cycloalkyl or heterocyclyl portion of the —C(O)cycloalkyl or —C(O)heterocyclyl are each optionally substituted with one or more R¹¹. In one embodiment, R² is —C(O)cycloalkyl and the cycloalkyl is cyclopropyl substituted with one R¹¹, wherein R¹¹ is C1-C3 alkyl or haloalkyl. In one embodiment, R² is —C(O)heterocyclyl, wherein the heterocyclyl is oxetanyl, tetrahydrofuranyl or tetrahydropyranyl.

In one embodiment, Q is a bond and the heterocyclyl is a bicyclic heterocyclyl. In certain embodiments, the bicyclic heterocyclyl is diazabicyclo[3.2.0]heptan-2-yl, (1R,5R)-2,6-diazabicyclo[3.2.0]heptan-2-yl, diazabicyclo[3.2.0]heptan-6-yl, (1R,5R)-2,6-diazabicyclo[3.2.0]heptan-6-yl, 6,7-dihydropyrazolo[1,5-a]pyrazin-5(4H)-yl, 5,6-dihydroimidazo[1,5-a]pyrazin-7(8H)-yl, 1,3-dimethyl-5,6-dihydroimidazo[1,5-a]pyrazin-7(8H)-yl or (R)-2-methylhexahydropyrrolo[1,2-a]pyrazin-6(2H)-one.

In yet another embodiment, Q is O and the heterocyclyl is azetidinyl, tetrahydrofuranyl, pyrrolidinyl, or piperdinyl.

In another embodiment when X is CR⁷ and R⁷ is hydrogen, R¹ is aryl optionally substituted with one or more R². In one embodiment, the aryl is phenyl optionally substituted with one or more R². In certain embodiments, the phenyl is substituted with one R², wherein R² is C1-C3 alkyl, alkoxy, hydroxyl or —N(R⁶)₂. In one embodiment, R² is —N(R⁶)₂ and each R⁶ is C1-C3 alkyl. In one embodiment, each C1-C3 alkyl is methyl. In other embodiments, R¹ is heteroaryl optionally substituted with one or more R². In one embodiment, the heteroaryl is pyrazolyl optionally substituted with one or more R². In one embodiment, the pyrazolyl is substituted with one R², wherein R² is C1-C3 alkyl, alkoxy, hydroxyl or —N(R⁶)₂. In one embodiment, R² is —N(R⁶)₂and each R⁶ is C1-C3 alkyl. In one embodiment, each C1-C3 alkyl is methyl.

In one embodiment for compounds of Formula (I), X is CR⁷ and R⁷ is alkoxy. In one embodiment, the alkoxy is methoxy. In certain embodiments wherein X is CR⁷ and R⁷ is alkoxy, R¹ is alkoxy. In one embodiment, the R⁷ alkoxy is methoxy and the R¹ alkoxy is methoxy.

In certain embodiments for compounds of Formula (I) wherein X is N or CR⁷, Y is heteroarylene. In one embodiment, the heteroarylene is thiophenylene.

In certain embodiments for compounds of Formula (I) wherein X is N or CR⁷, Y is a bond.

In certain embodiments for compounds of Formula (I), R⁴ is aryl or heteroaryl, each optionally substituted with one or more R⁵. In one embodiment, R⁴ is aryl optionally substituted with one or more R⁵. In one embodiment, the aryl is phenyl optionally substituted with one or more R⁵. In certain embodiments, the phenyl is substituted with one R⁵, wherein R⁵ is C1-C4 alkyl, haloalkyl or -L-N(R⁶)₂.

In one embodiment, R⁵ is -L-N(R⁶)₂, wherein L is methylene and one R⁶ is hydrogen and the second R⁶ is C1-C3 alkyl. In one embodiment, the C1-C3 alkyl is methyl. In another embodiment, R⁵ is -L-N(R⁶)₂, wherein L is methylene and each R⁶ is C1-C3 alkyl. In one embodiment, each of the C1-C3 alkyl is methyl.

In certain embodiments wherein R⁴ is aryl, R⁴ is phenyl substituted with two R⁵, wherein one R⁵ is C1-C4 alkyl and the second R⁵ is haloalkyl. In one embodiment, the C1-C4 alkyl is methyl and the haloalkyl is trifluoromethyl. In certain embodiments, R⁴ is phenyl substituted with two R⁵, wherein one R⁵ is C1-C4 alkyl and the second R⁵ is -L-N(R⁶)₂. In one embodiment, L is methylene and each R⁶ is C1-C3 alkyl.

In one embodiment for compounds of Formula (I), R³ is hydrogen.

In certain embodiments for compounds of Formula (I), R³ is C1-C6 alkyl optionally substituted with one or more R⁹. In one embodiment, the C1-C6 alkyl is methyl, ethyl or isopropyl.

In certain embodiments for compounds of Formula (I), R³ is alkoxy. In one embodiment, the alkoxy is methoxy.

In certain embodiments for compounds of Formula (I), R³ is haloalkyl. In one embodiment, the haloalkyl is trifluoromethyl.

In certain embodiments for compounds of Formula (I), R³ is cycloalkyl optionally substituted with one or more R⁹. In one embodiment, the cycloalkyl is cyclopropyl. In one embodiment, the cycloalkyl is substituted with one R⁹, wherein the one R⁹ is halogen amino, hydroxyl or alkoxy.

In certain embodiments for compounds of Formula (I), R³ is —N(R¹⁰)₂. In one embodiment, each R¹⁰ is C1-C3 alkyl. In certain embodiments, each C1-C3 alkyl is methyl.

In certain embodiments for compounds of Formula (I), R³ is -L-N(R¹⁰)₂. In one embodiment, each R¹⁰ is C1-C3 alkyl. In certain embodiments, each C1-C3 alkyl is methyl.

In certain embodiments for compounds of Formula (I), R³ is heterocyclyl, aryl, or heteroaryl, wherein the heterocyclyl, the aryl, and the heteroaryl are each optionally substituted with one or more R⁹.

In certain embodiments for compounds of Formula (I), R¹ is C1-C2 alkyl. In one embodiment, the C1-C2 alkyl is methyl.

In certain embodiments for compounds of Formula (I), R⁸ is haloC1-C2 alkyl. In one embodiment, the haloC1-C2 alkyl is fluoromethyl, difluoromethyl or trifluoromethyl.

In one embodiment, the compound of Formula (I) is:

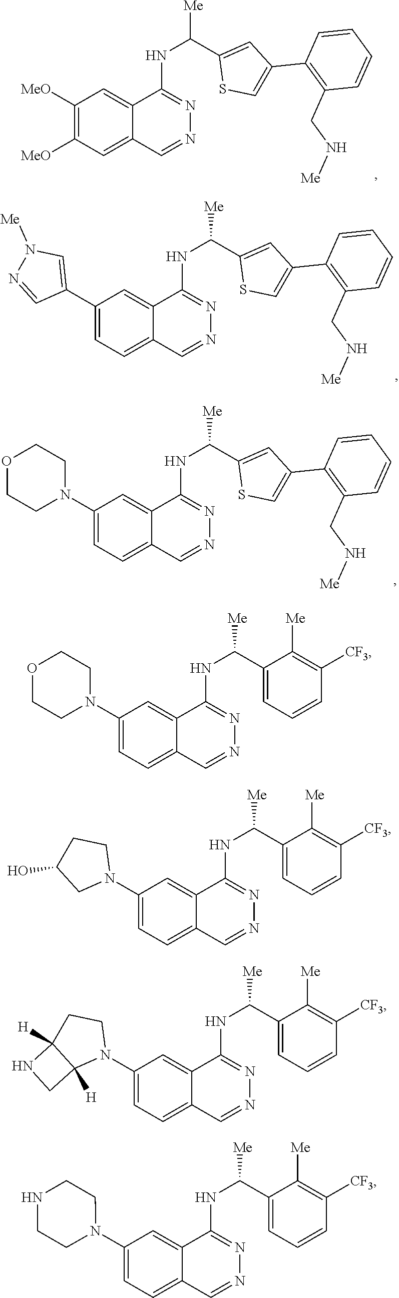

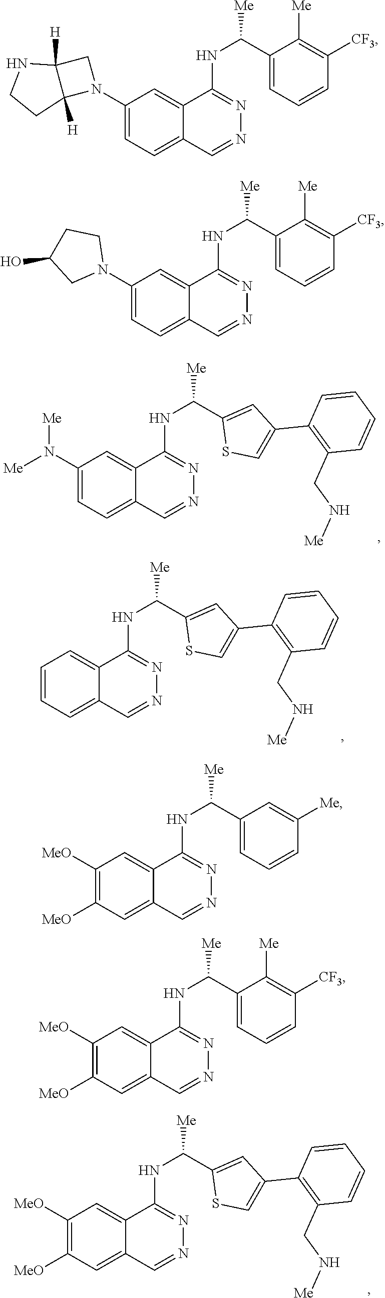

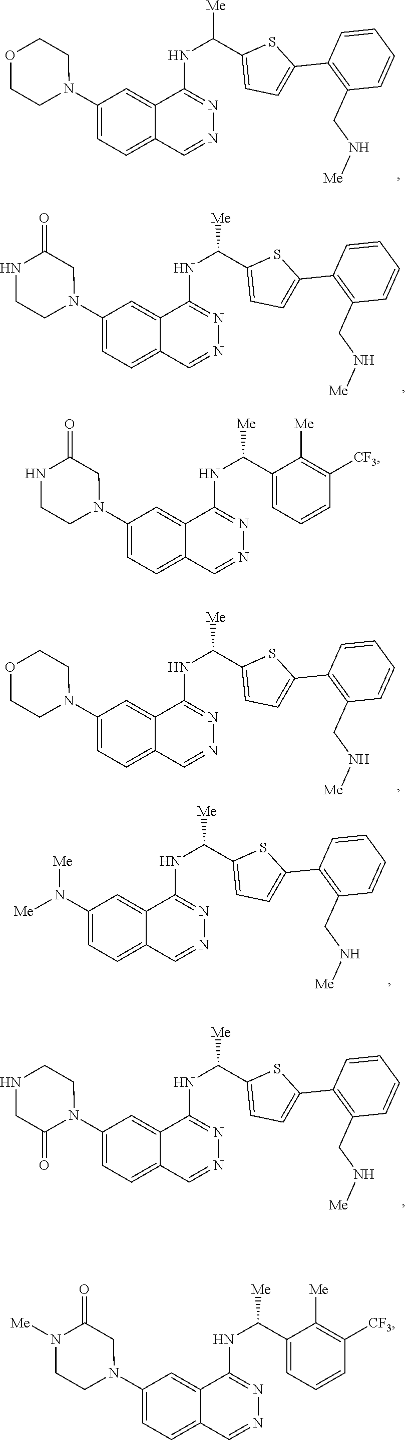

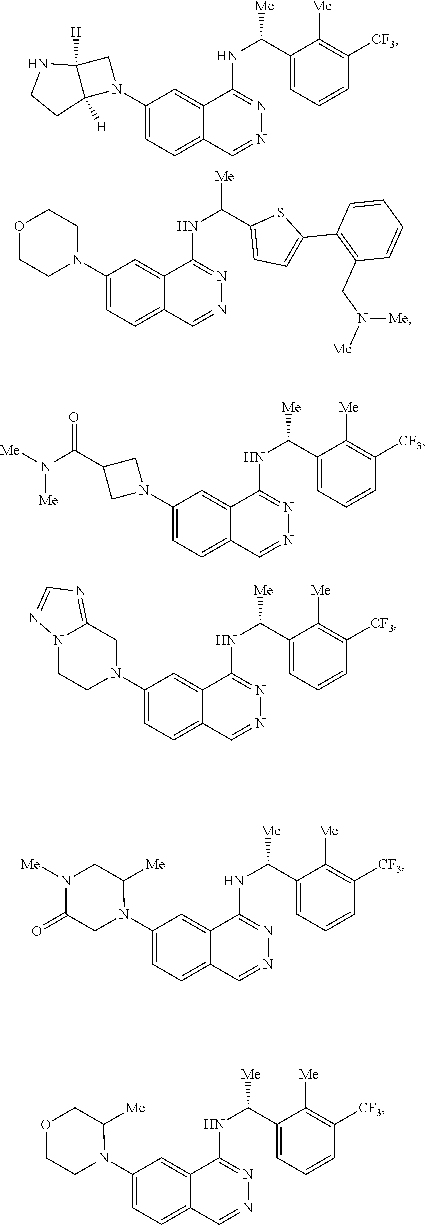

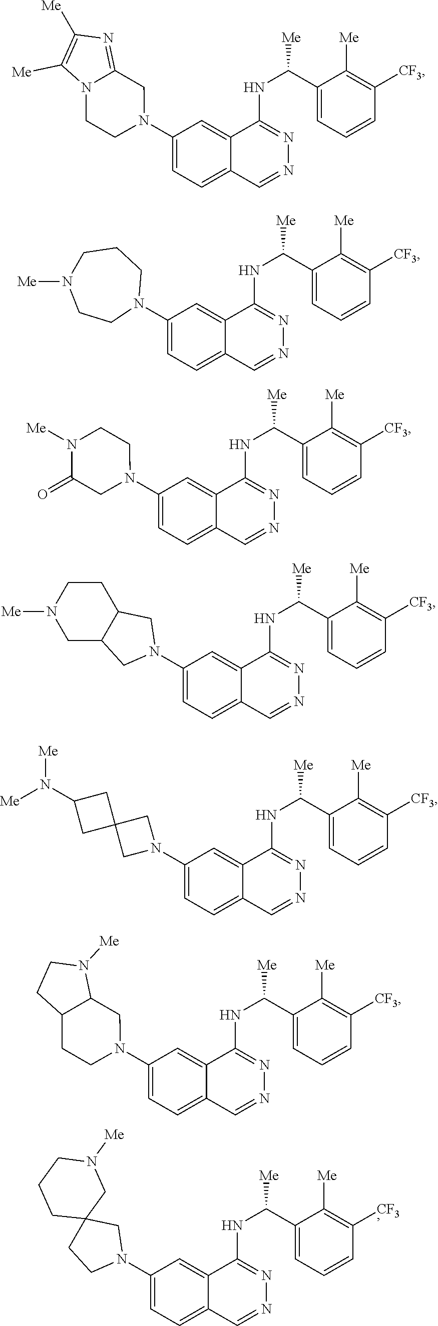

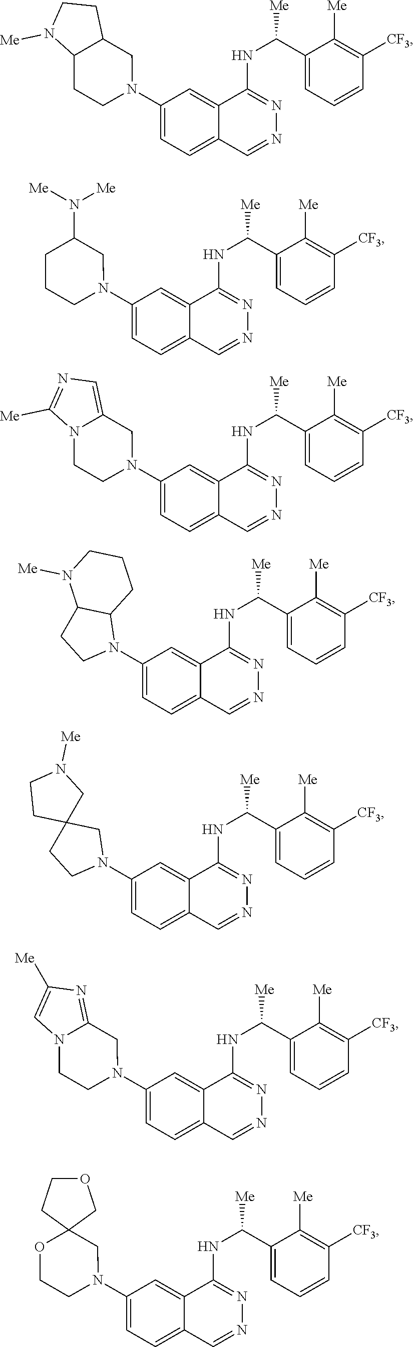

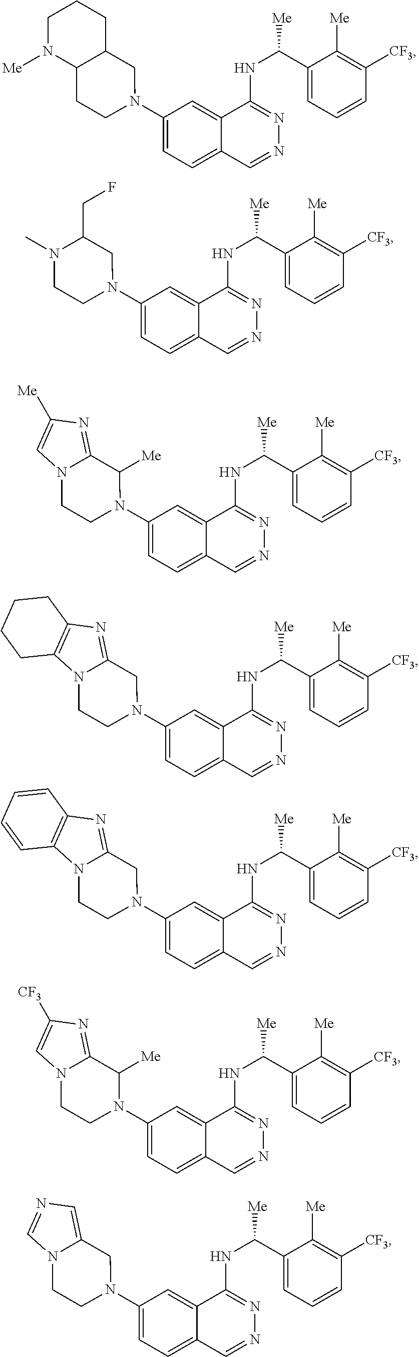

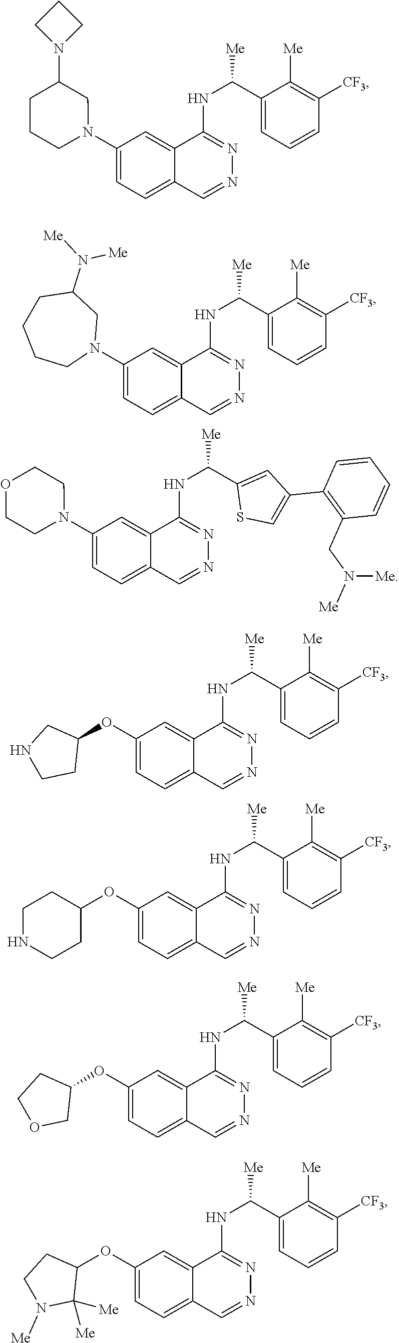

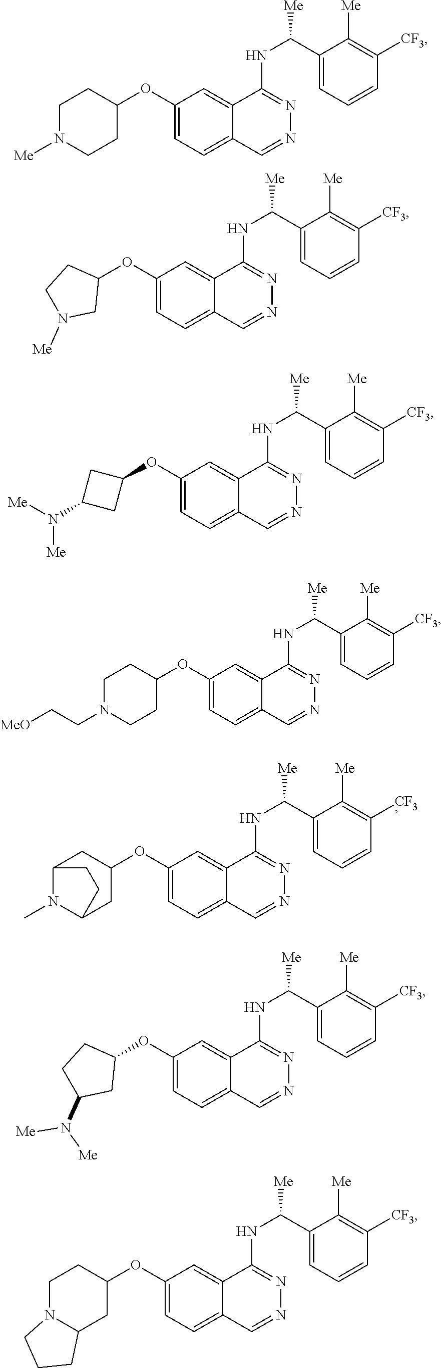

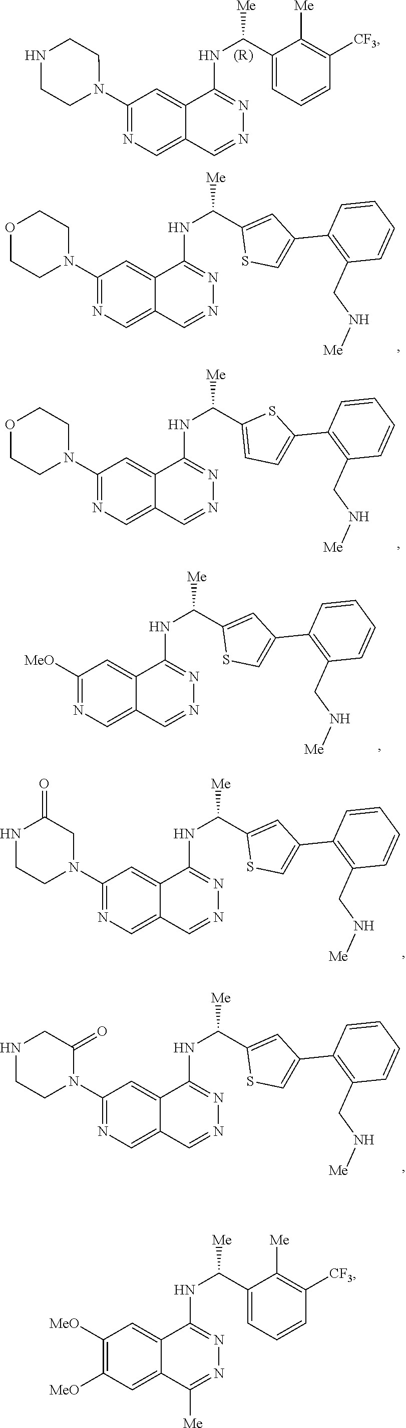

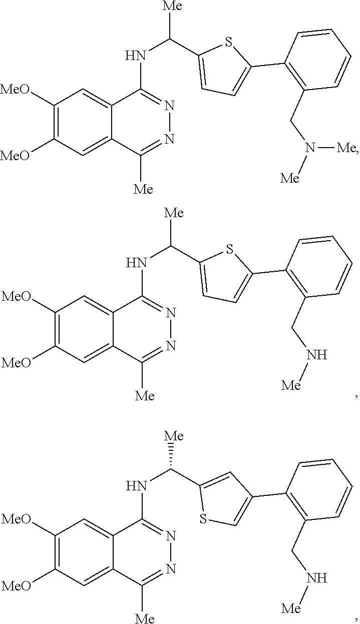

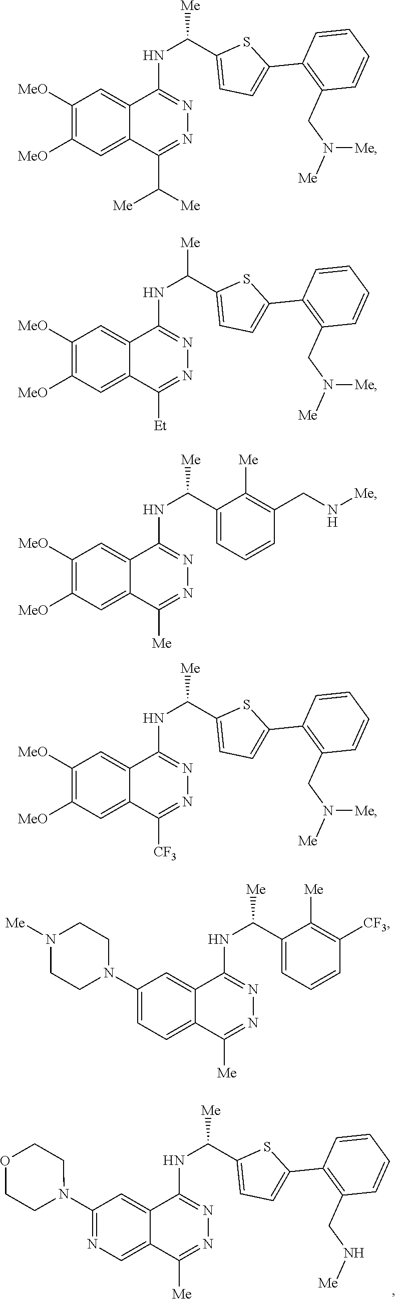

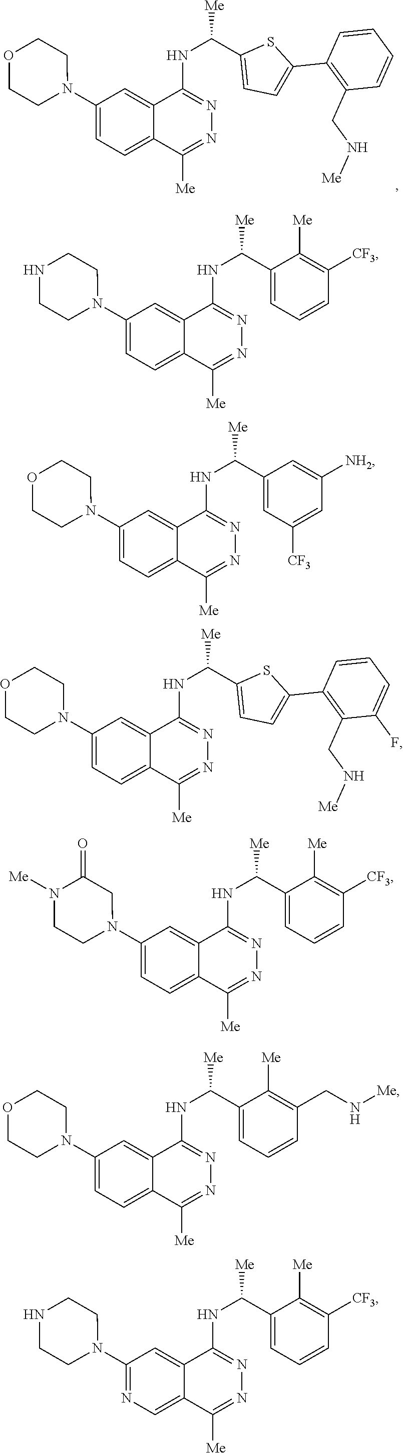

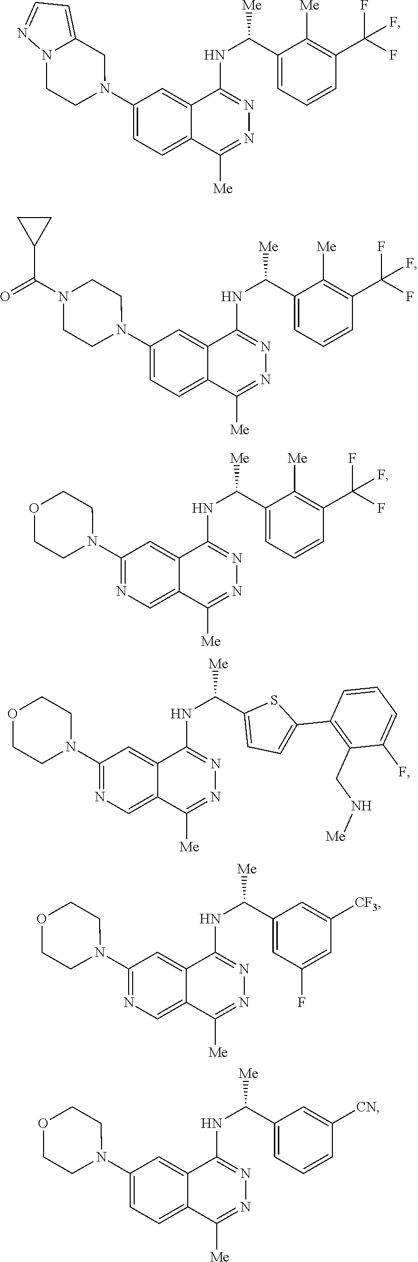

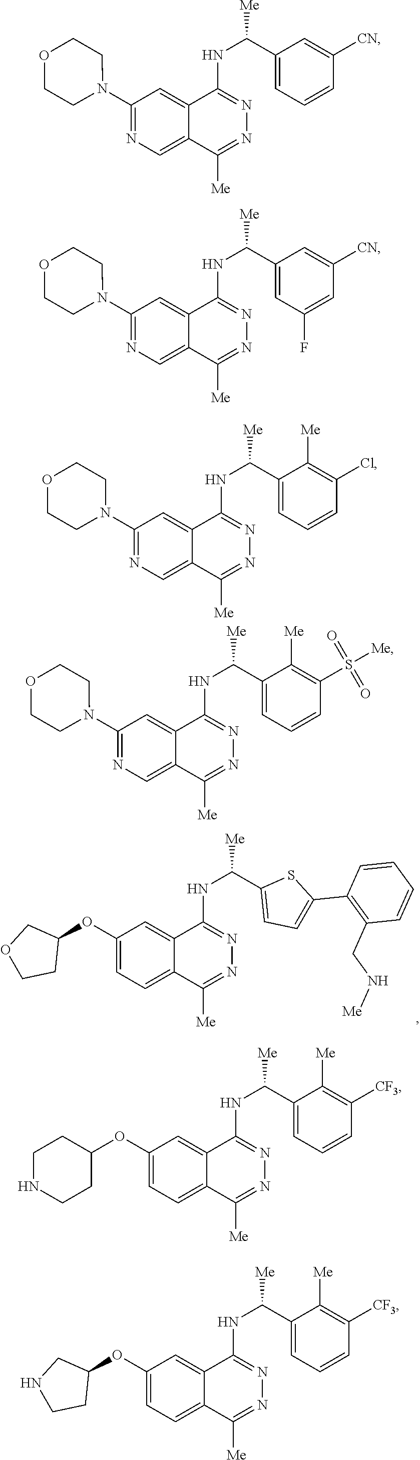

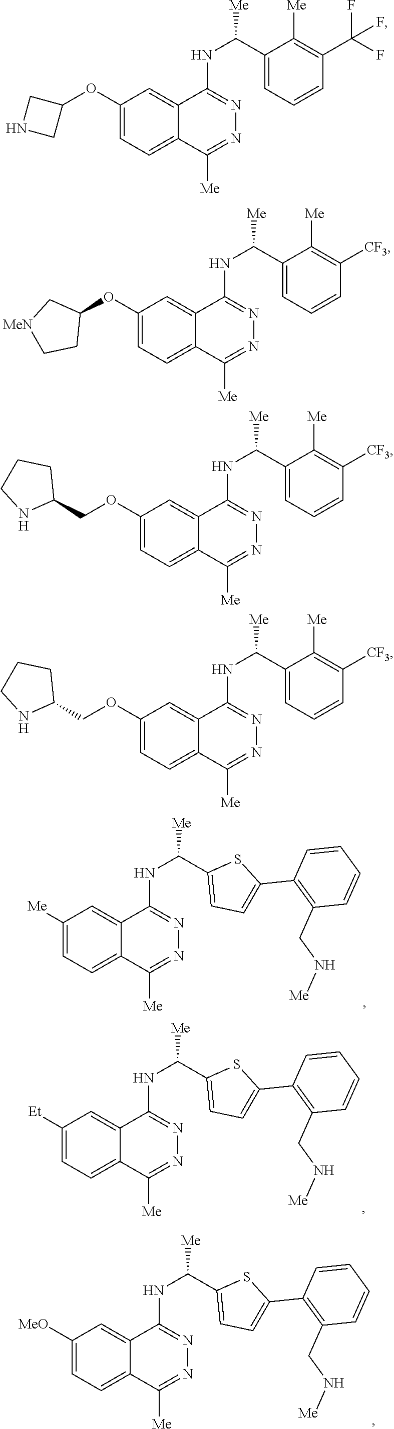

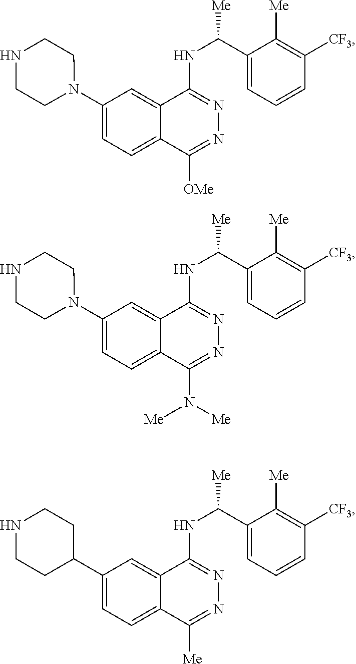

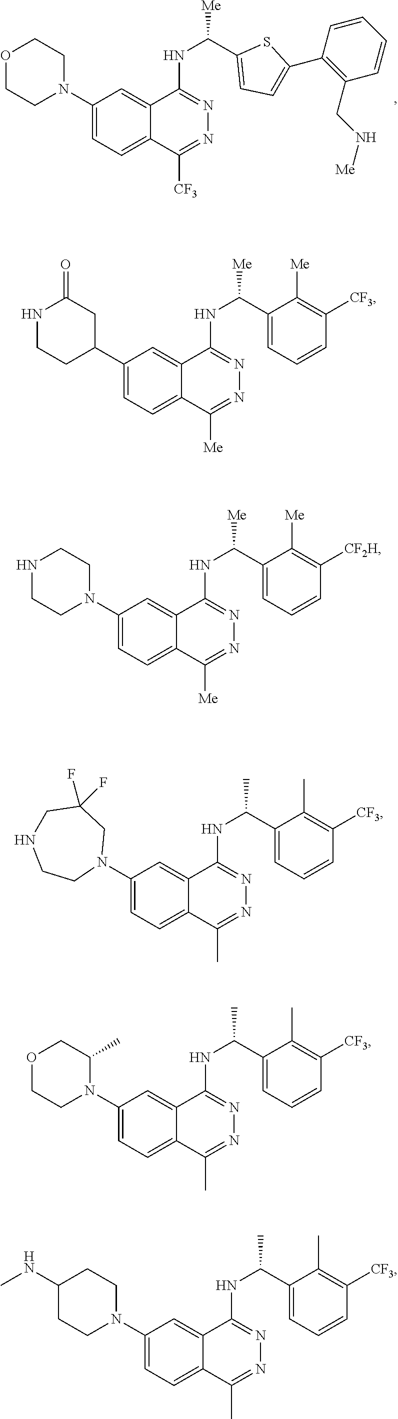 [SMILES: CNCc1ccccc1-c1ccc([C@@H](C)Nc2nnc(C(F)(F)F)c3ccc(N4CCOCC4)cc23)s1 ; Cc1c([C@@H](C)Nc2nnc(C)c3ccc(C4CCNC(=O)C4)cc23)cccc1C(F)(F)F ; Cc1c(C(F)F)cccc1[C@@H](C)Nc1nnc(C)c2ccc(N3CCNCC3)cc12 ; Cc1c([C@@H](C)Nc2nnc(C)c3ccc(N4CCNCC(F)(F)C4)cc23)cccc1C(F)(F)F ; Cc1c([C@@H](C)Nc2nnc(C)c3ccc(N4CCOC[C@@H]4C)cc23)cccc1C(F)(F)F ; CNC1CCN(c2ccc3c(C)nnc(N[C@H](C)c4cccc(C(F)(F)F)c4C)c3c2)CC1]

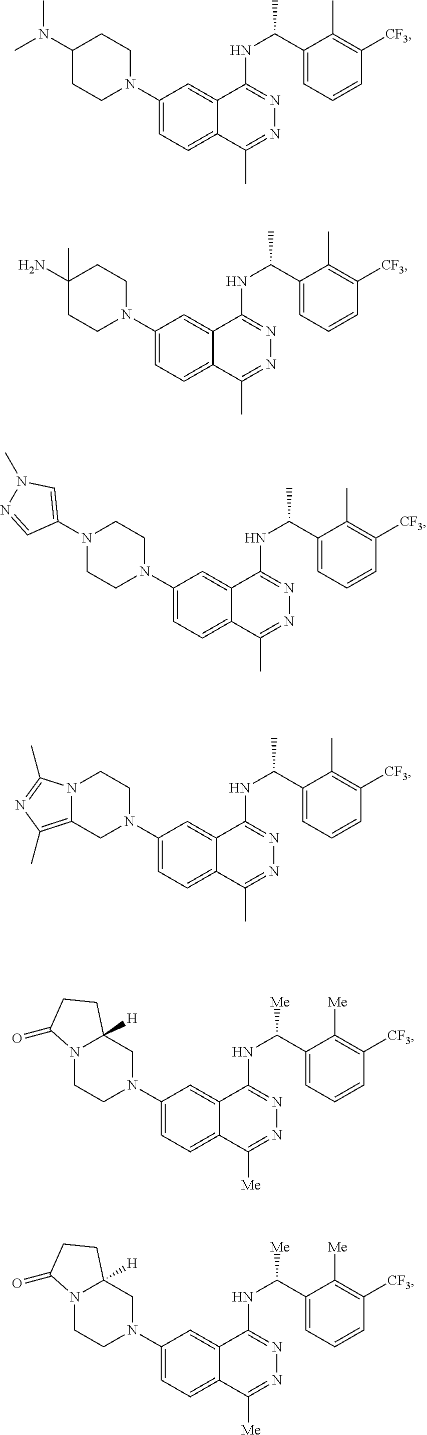 [SMILES: Cc1c([C@@H](C)Nc2nnc(C)c3ccc(N4CCC(N(C)C)CC4)cc23)cccc1C(F)(F)F ; Cc1c([C@@H](C)Nc2nnc(C)c3ccc(N4CCC(C)(N)CC4)cc23)cccc1C(F)(F)F ; Cc1c([C@@H](C)Nc2nnc(C)c3ccc(N4CCN(c5cnn(C)c5)CC4)cc23)cccc1C(F)(F)F ; Cc1nc(C)n2c1CN(c1ccc3c(C)nnc(N[C@H](C)c4cccc(C(F)(F)F)c4C)c3c1)CC2 ; Cc1c([C@@H](C)Nc2nnc(C)c3ccc(N4CCN5C(=O)CC[C@@H]5C4)cc23)cccc1C(F)(F)F ; Cc1c([C@@H](C)Nc2nnc(C)c3ccc(N4CCN5C(=O)CC[C@H]5C4)cc23)cccc1C(F)(F)F]

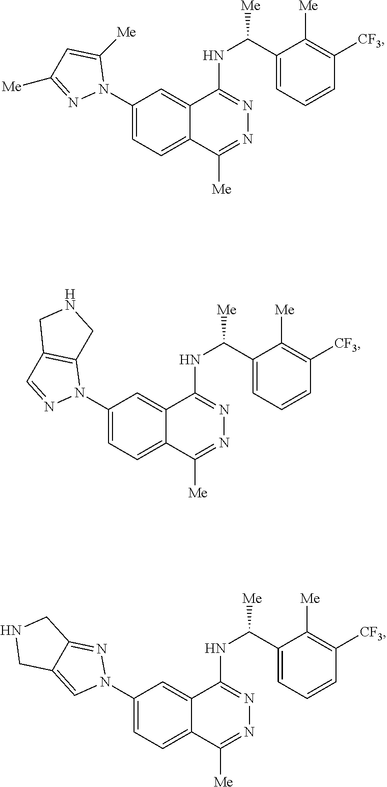 [SMILES: Cc1cc(C)n(-c2ccc3c(C)nnc(N[C@H](C)c4cccc(C(F)(F)F)c4C)c3c2)n1 ; Cc1c([C@@H](C)Nc2nnc(C)c3ccc(-n4ncc5c4CNC5)cc23)cccc1C(F)(F)F ; Cc1c([C@@H](C)Nc2nnc(C)c3ccc(-n4cc5c(n4)CNC5)cc23)cccc1C(F)(F)F]

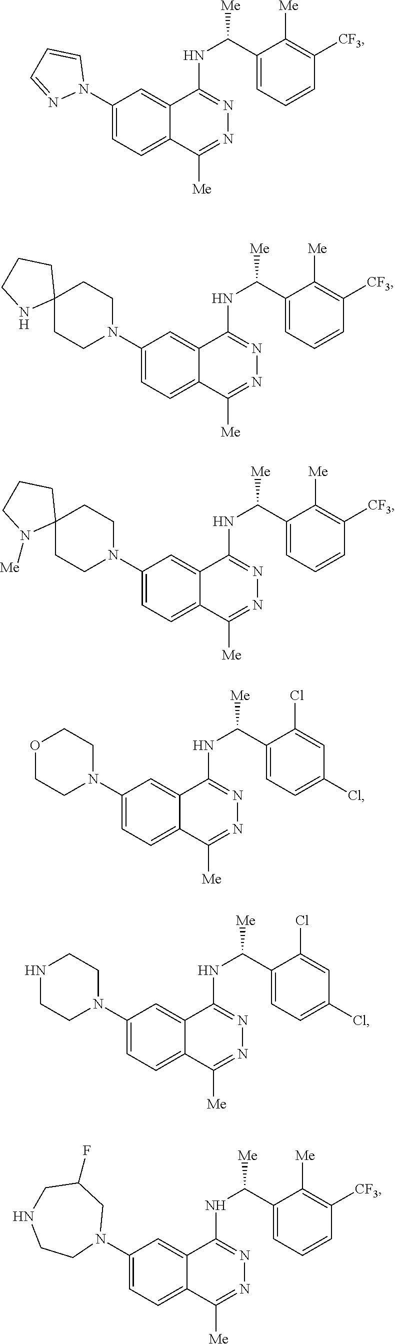

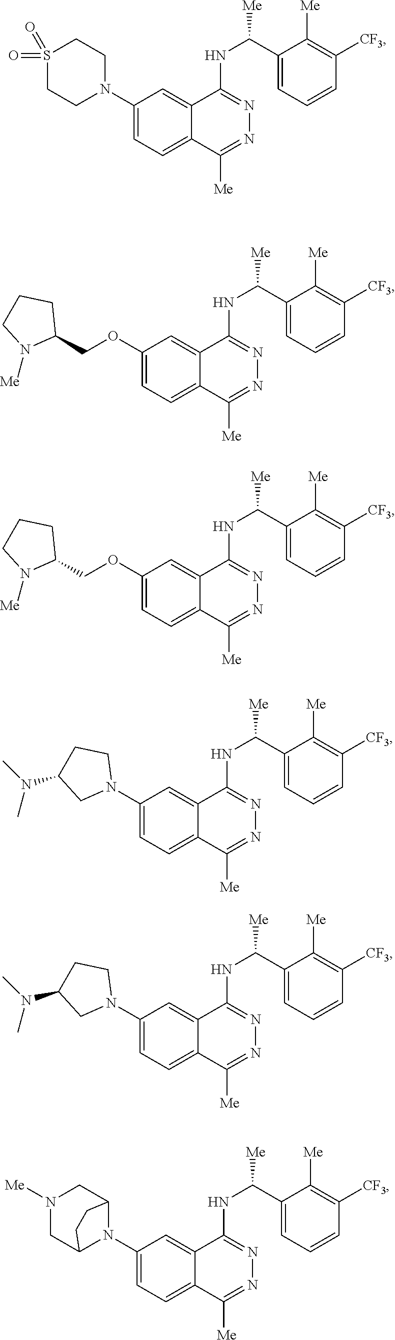

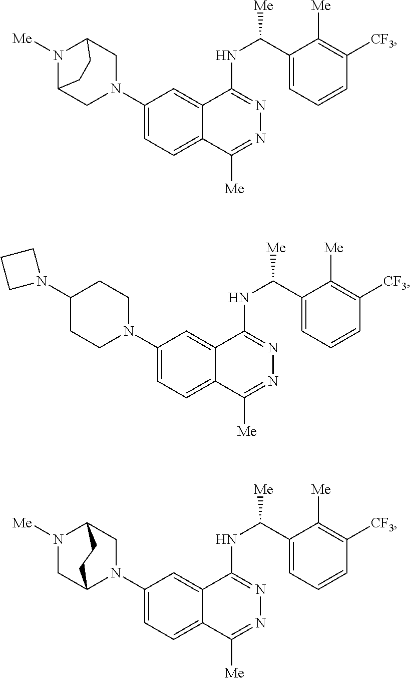 [SMILES: Cc1c([C@@H](C)Nc2nnc(C)c3ccc(N4CC5CCC(C4)N5C)cc23)cccc1C(F)(F)F ; Cc1c([C@@H](C)Nc2nnc(C)c3ccc(N4CCC(N5CCC5)CC4)cc23)cccc1C(F)(F)F ; Cc1c([C@@H](C)Nc2nnc(C)c3ccc(N4C[C@@H]5CC[C@H]4CN5C)cc23)cccc1C(F)(F)F]

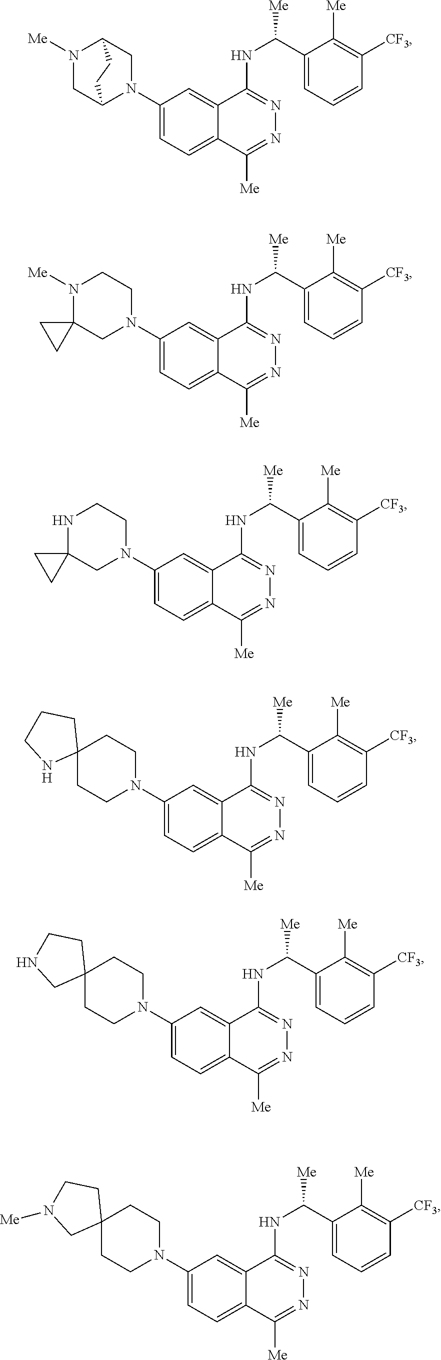 [SMILES: Cc1c([C@@H](C)Nc2nnc(C)c3ccc(N4C[C@H]5CC[C@@H]4CN5C)cc23)cccc1C(F)(F)F ; Cc1c([C@@H](C)Nc2nnc(C)c3ccc(N4CCN(C)C5(CC5)C4)cc23)cccc1C(F)(F)F ; Cc1c([C@@H](C)Nc2nnc(C)c3ccc(N4CCNC5(CC5)C4)cc23)cccc1C(F)(F)F ; Cc1c([C@@H](C)Nc2nnc(C)c3ccc(N4CCC5(CCCN5)CC4)cc23)cccc1C(F)(F)F ; Cc1c([C@@H](C)Nc2nnc(C)c3ccc(N4CCC5(CCNC5)CC4)cc23)cccc1C(F)(F)F ; Cc1c([C@@H](C)Nc2nnc(C)c3ccc(N4CCC5(CCN(C)C5)CC4)cc23)cccc1C(F)(F)F]

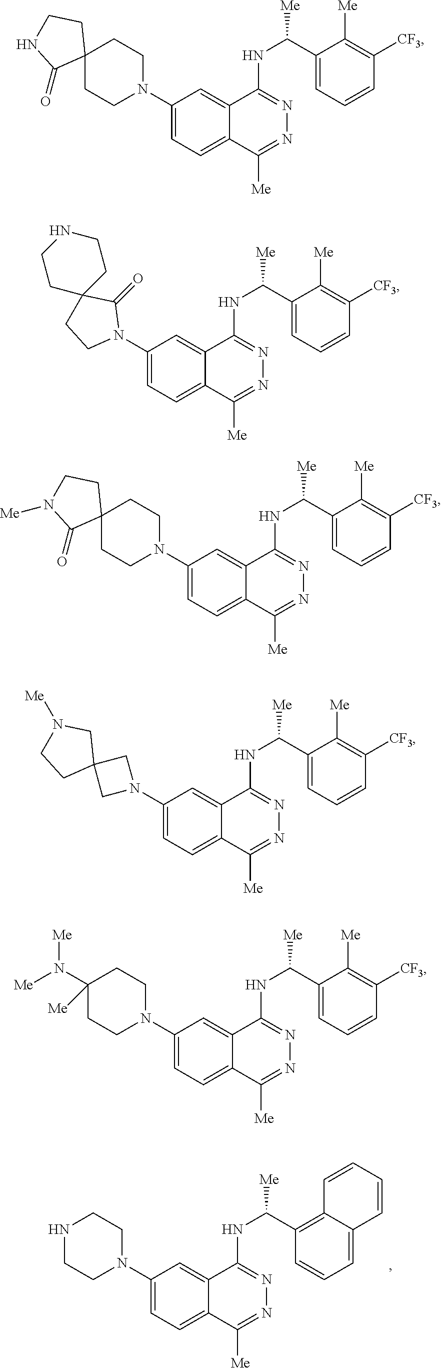 [SMILES: Cc1c([C@@H](C)Nc2nnc(C)c3ccc(N4CCC5(CCNC5=O)CC4)cc23)cccc1C(F)(F)F ; Cc1c([C@@H](C)Nc2nnc(C)c3ccc(N4CCC5(CCNCC5)C4=O)cc23)cccc1C(F)(F)F ; Cc1c([C@@H](C)Nc2nnc(C)c3ccc(N4CCC5(CCN(C)C5=O)CC4)cc23)cccc1C(F)(F)F ; Cc1c([C@@H](C)Nc2nnc(C)c3ccc(N4CC5(CCN(C)C5)C4)cc23)cccc1C(F)(F)F ; Cc1c([C@@H](C)Nc2nnc(C)c3ccc(N4CCC(C)(N(C)C)CC4)cc23)cccc1C(F)(F)F ; Cc1nnc(N[C@H](C)c2cccc3ccccc23)c2cc(N3CCNCC3)ccc12]

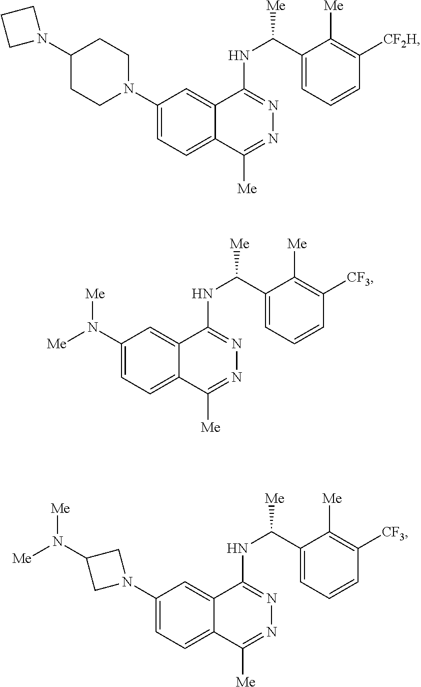 [SMILES: Cc1c(C(F)F)cccc1[C@@H](C)Nc1nnc(C)c2ccc(N3CCC(N4CCC4)CC3)cc12 ; Cc1c([C@@H](C)Nc2nnc(C)c3ccc(N(C)C)cc23)cccc1C(F)(F)F ; Cc1c([C@@H](C)Nc2nnc(C)c3ccc(N4CC(N(C)C)C4)cc23)cccc1C(F)(F)F]

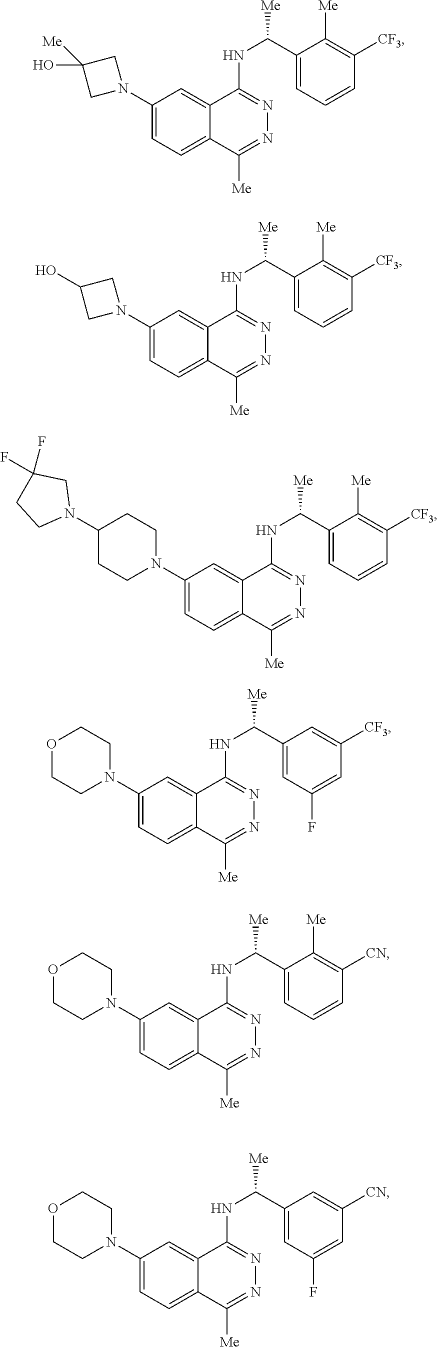

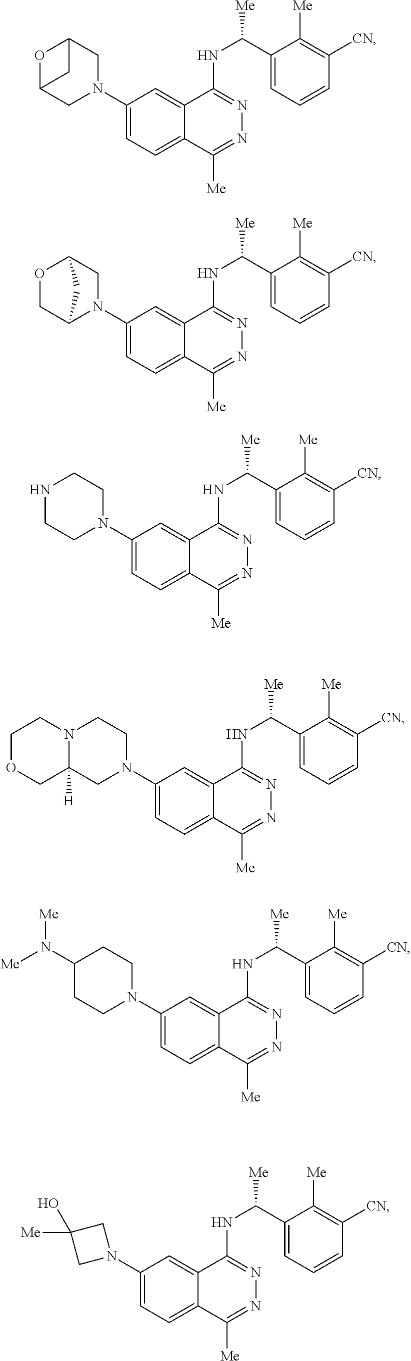

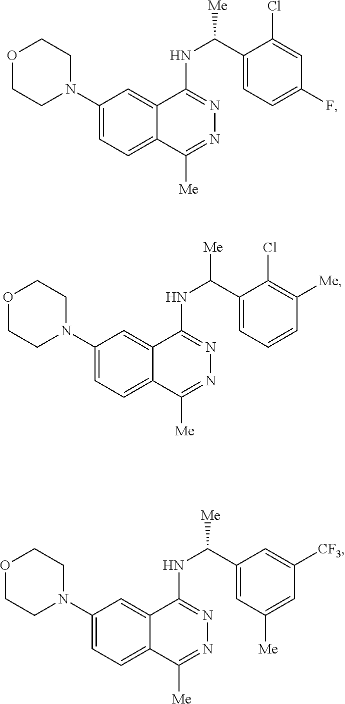 [SMILES: Cc1nnc(N[C@H](C)c2ccc(F)cc2Cl)c2cc(N3CCOCC3)ccc12 ; Cc1cccc(C(C)Nc2nnc(C)c3ccc(N4CCOCC4)cc23)c1Cl ; Cc1cc([C@@H](C)Nc2nnc(C)c3ccc(N4CCOCC4)cc23)cc(C(F)(F)F)c1]

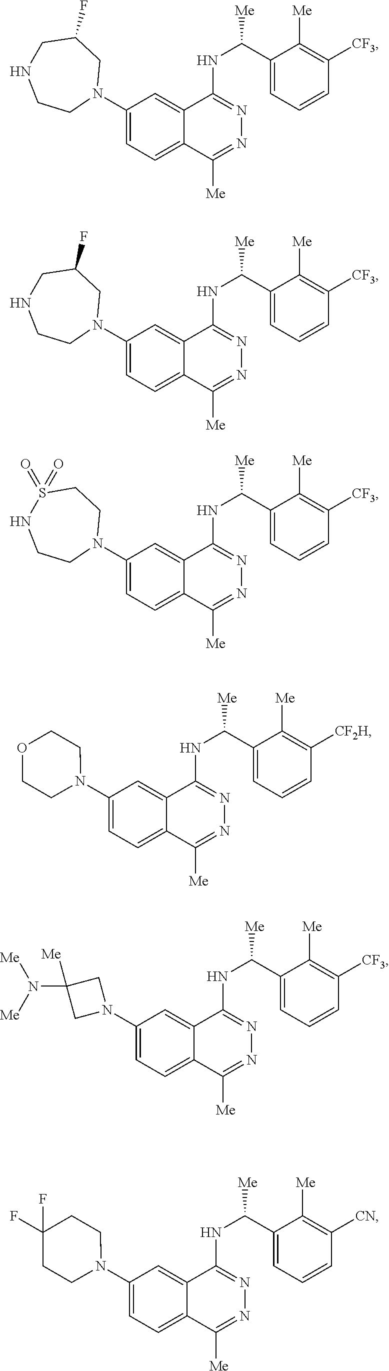

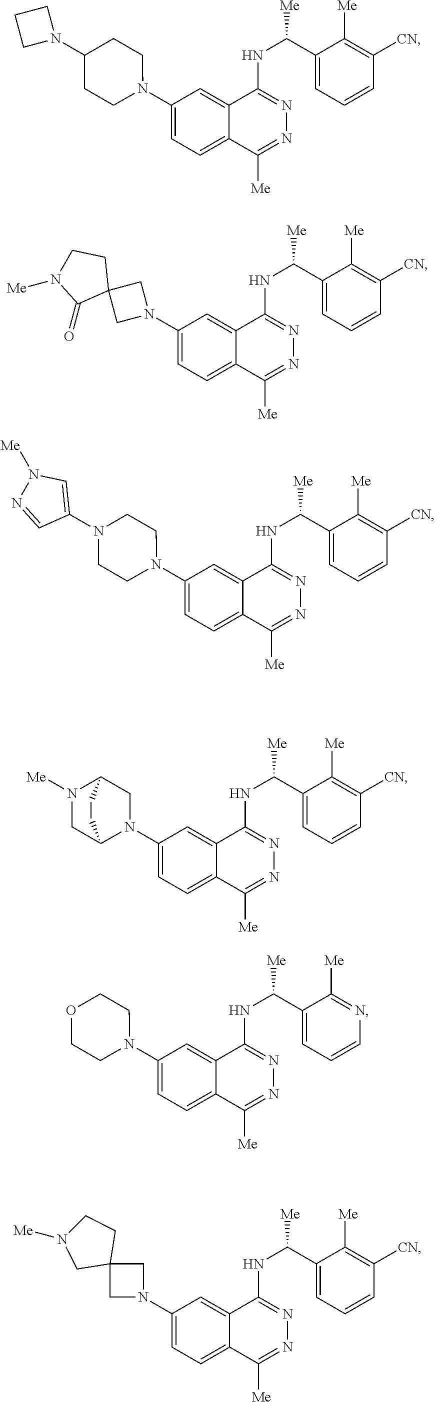

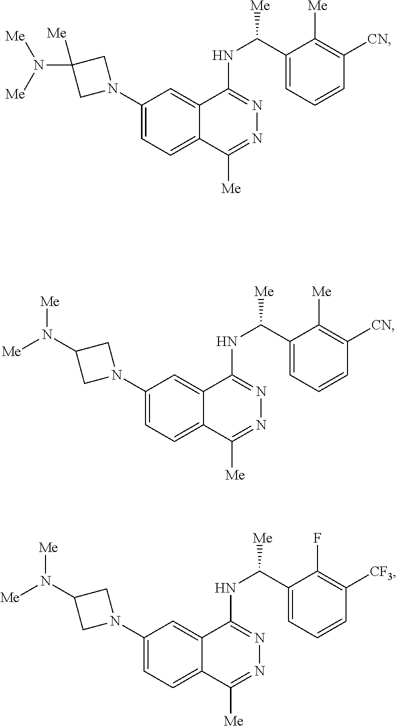 [SMILES: Cc1c(C#N)cccc1[C@@H](C)Nc1nnc(C)c2ccc(N3CC(C)(N(C)C)C3)cc12 ; Cc1c(C#N)cccc1[C@@H](C)Nc1nnc(C)c2ccc(N3CC(N(C)C)C3)cc12 ; Cc1nnc(N[C@H](C)c2cccc(C(F)(F)F)c2F)c2cc(N3CC(N(C)C)C3)ccc12]

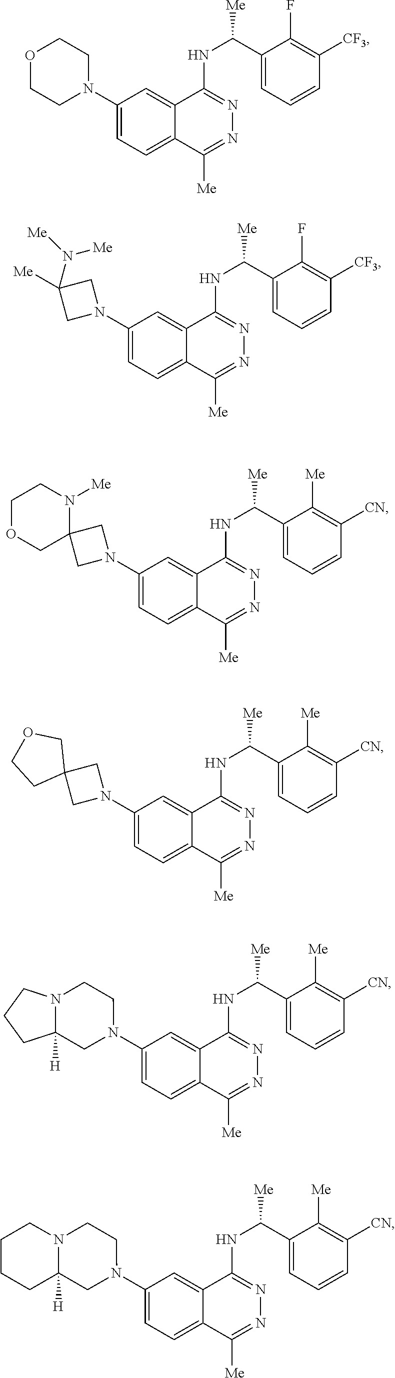 [SMILES: Cc1nnc(N[C@H](C)c2cccc(C(F)(F)F)c2F)c2cc(N3CCOCC3)ccc12 ; Cc1nnc(N[C@H](C)c2cccc(C(F)(F)F)c2F)c2cc(N3CC(C)(N(C)C)C3)ccc12 ; Cc1c(C#N)cccc1[C@@H](C)Nc1nnc(C)c2ccc(N3CC4(COCCN4C)C3)cc12 ; Cc1c(C#N)cccc1[C@@H](C)Nc1nnc(C)c2ccc(N3CC4(CCOC4)C3)cc12 ; Cc1c(C#N)cccc1[C@@H](C)Nc1nnc(C)c2ccc(N3CCN4CCC[C@@H]4C3)cc12 ; Cc1c(C#N)cccc1[C@@H](C)Nc1nnc(C)c2ccc(N3CCN4CCCC[C@@H]4C3)cc12]

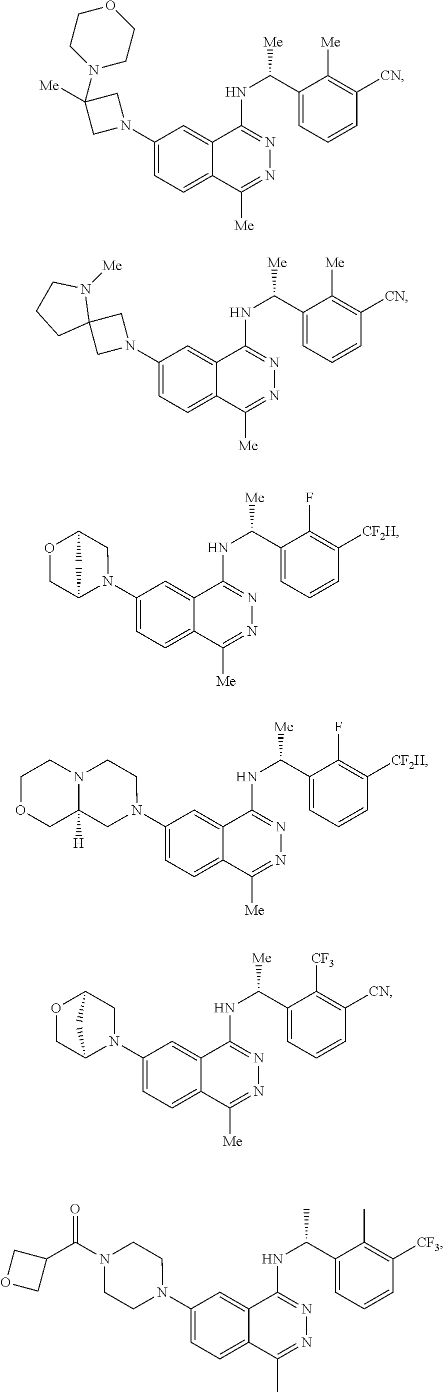 [SMILES: Cc1c(C#N)cccc1[C@@H](C)Nc1nnc(C)c2ccc(N3CC(C)(N4CCOCC4)C3)cc12 ; Cc1c(C#N)cccc1[C@@H](C)Nc1nnc(C)c2ccc(N3CC4(CCCN4C)C3)cc12 ; Cc1nnc(N[C@H](C)c2cccc(C(F)F)c2F)c2cc(N3C[C@H]4C[C@@H]3CO4)ccc12 ; Cc1nnc(N[C@H](C)c2cccc(C(F)F)c2F)c2cc(N3CCN4CCOC[C@@H]4C3)ccc12 ; Cc1nnc(N[C@H](C)c2cccc(C#N)c2C(F)(F)F)c2cc(N3C[C@H]4C[C@@H]3CO4)ccc12 ; Cc1c([C@@H](C)Nc2nnc(C)c3ccc(N4CCN(C(=O)C5COC5)CC4)cc23)cccc1C(F)(F)F]

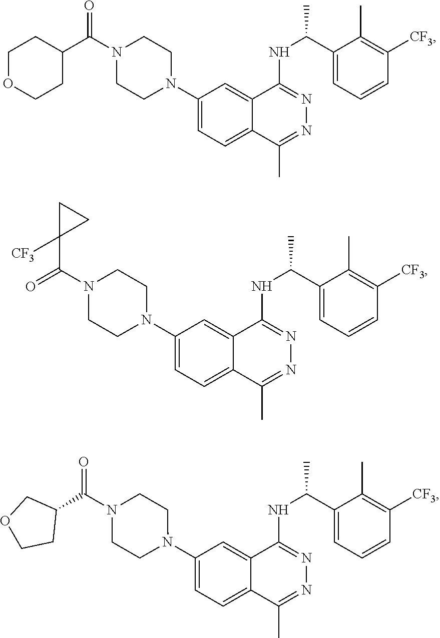 [SMILES: Cc1c([C@@H](C)Nc2nnc(C)c3ccc(N4CCN(C(=O)C5CCOCC5)CC4)cc23)cccc1C(F)(F)F ; Cc1c([C@@H](C)Nc2nnc(C)c3ccc(N4CCN(C(=O)C5(C(F)(F)F)CC5)CC4)cc23)cccc1C(F)(F)F ; Cc1c([C@@H](C)Nc2nnc(C)c3ccc(N4CCN(C(=O)[C@@H]5CCOC5)CC4)cc23)cccc1C(F)(F)F]

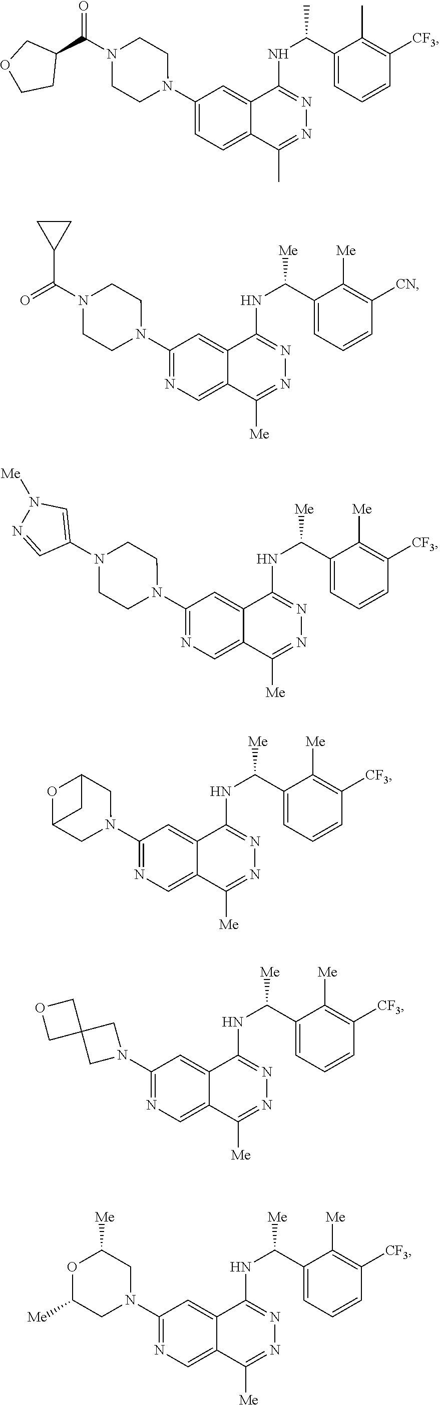 [SMILES: Cc1c([C@@H](C)Nc2nnc(C)c3ccc(N4CCN(C(=O)[C@H]5CCOC5)CC4)cc23)cccc1C(F)(F)F ; Cc1c(C#N)cccc1[C@@H](C)Nc1nnc(C)c2cnc(N3CCN(C(=O)C4CC4)CC3)cc12 ; Cc1c([C@@H](C)Nc2nnc(C)c3cnc(N4CCN(c5cnn(C)c5)CC4)cc23)cccc1C(F)(F)F ; Cc1c([C@@H](C)Nc2nnc(C)c3cnc(N4CC5CC(C4)O5)cc23)cccc1C(F)(F)F ; Cc1c([C@@H](C)Nc2nnc(C)c3cnc(N4CC5(COC5)C4)cc23)cccc1C(F)(F)F ; Cc1c([C@@H](C)Nc2nnc(C)c3cnc(N4C[C@@H](C)O[C@@H](C)C4)cc23)cccc1C(F)(F)F]

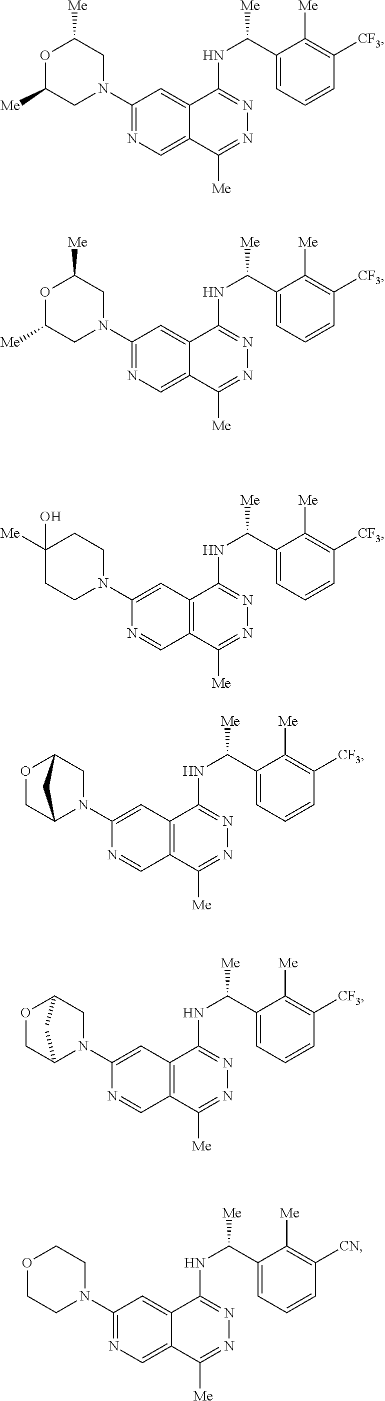

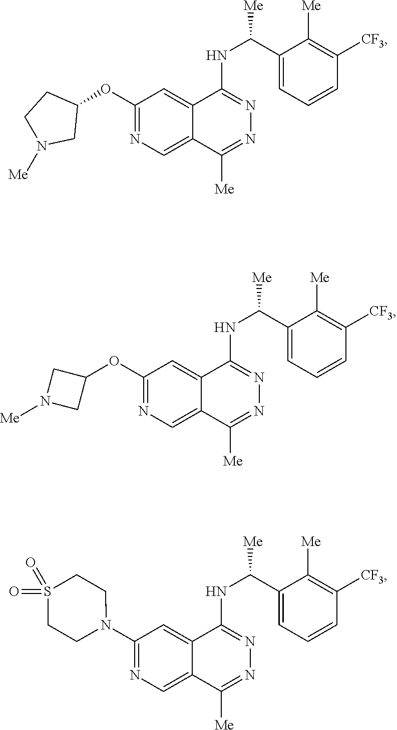 [SMILES: Cc1c([C@@H](C)Nc2nnc(C)c3cnc(O[C@H]4CCN(C)C4)cc23)cccc1C(F)(F)F ; Cc1c([C@@H](C)Nc2nnc(C)c3cnc(OC4CN(C)C4)cc23)cccc1C(F)(F)F ; Cc1c([C@@H](C)Nc2nnc(C)c3cnc(N4CCS(=O)(=O)CC4)cc23)cccc1C(F)(F)F]

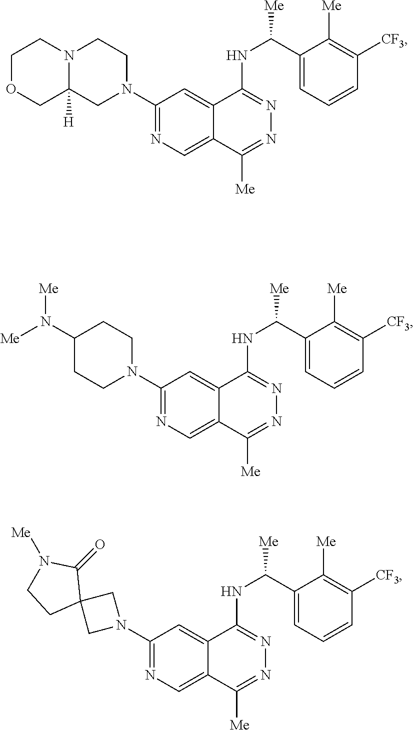 [SMILES: Cc1c([C@@H](C)Nc2nnc(C)c3cnc(N4CCN5CCOC[C@@H]5C4)cc23)cccc1C(F)(F)F ; Cc1c([C@@H](C)Nc2nnc(C)c3cnc(N4CCC(N(C)C)CC4)cc23)cccc1C(F)(F)F ; Cc1c([C@@H](C)Nc2nnc(C)c3cnc(N4CC5(CCN(C)C5=O)C4)cc23)cccc1C(F)(F)F]

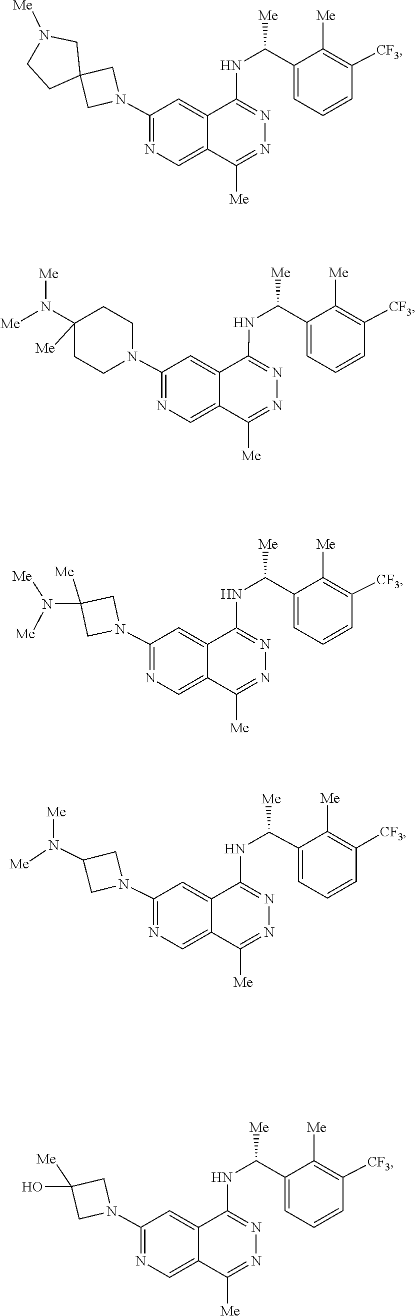 [SMILES: Cc1c([C@@H](C)Nc2nnc(C)c3cnc(N4CC5(CCN(C)C5)C4)cc23)cccc1C(F)(F)F ; Cc1c([C@@H](C)Nc2nnc(C)c3cnc(N4CCC(C)(N(C)C)CC4)cc23)cccc1C(F)(F)F ; Cc1c([C@@H](C)Nc2nnc(C)c3cnc(N4CC(C)(N(C)C)C4)cc23)cccc1C(F)(F)F ; Cc1c([C@@H](C)Nc2nnc(C)c3cnc(N4CC(N(C)C)C4)cc23)cccc1C(F)(F)F ; Cc1c([C@@H](C)Nc2nnc(C)c3cnc(N4CC(C)(O)C4)cc23)cccc1C(F)(F)F]

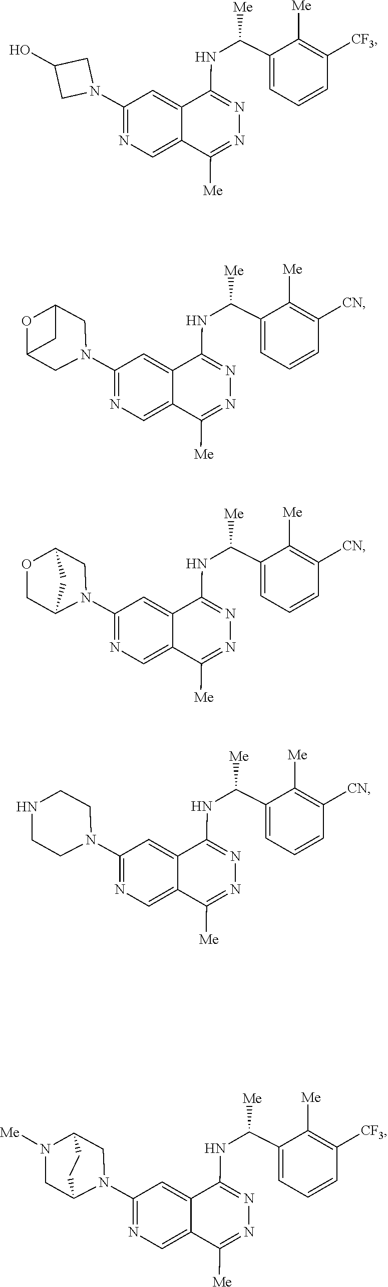 [SMILES: Cc1c([C@@H](C)Nc2nnc(C)c3cnc(N4CC(O)C4)cc23)cccc1C(F)(F)F ; Cc1c(C#N)cccc1[C@@H](C)Nc1nnc(C)c2cnc(N3CC4CC(C3)O4)cc12 ; Cc1c(C#N)cccc1[C@@H](C)Nc1nnc(C)c2cnc(N3C[C@H]4C[C@@H]3CO4)cc12 ; Cc1c(C#N)cccc1[C@@H](C)Nc1nnc(C)c2cnc(N3CCNCC3)cc12 ; Cc1c([C@@H](C)Nc2nnc(C)c3cnc(N4C[C@H]5CC[C@@H]4CN5C)cc23)cccc1C(F)(F)F]

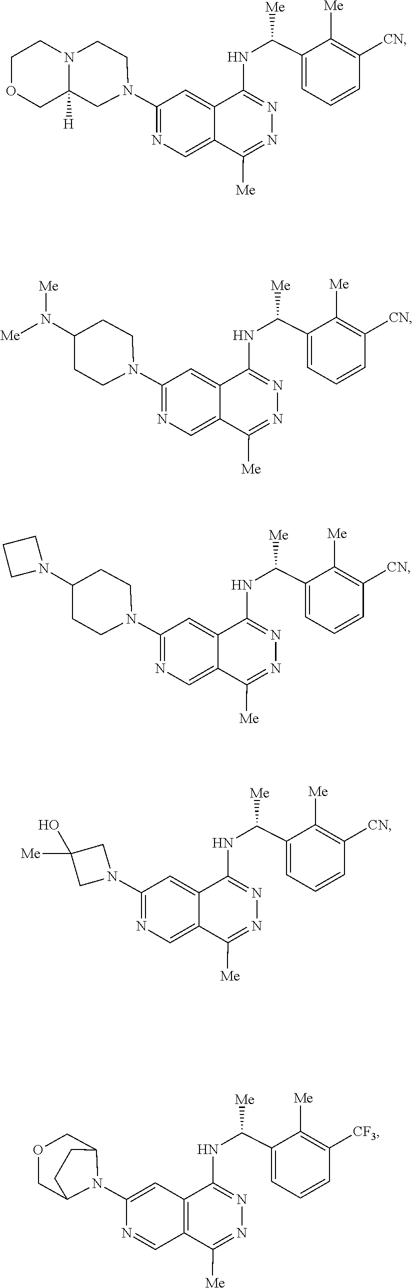 [SMILES: Cc1c(C#N)cccc1[C@@H](C)Nc1nnc(C)c2cnc(N3CCN4CCOC[C@@H]4C3)cc12 ; Cc1c(C#N)cccc1[C@@H](C)Nc1nnc(C)c2cnc(N3CCC(N(C)C)CC3)cc12 ; Cc1c(C#N)cccc1[C@@H](C)Nc1nnc(C)c2cnc(N3CCC(N4CCC4)CC3)cc12 ; Cc1c(C#N)cccc1[C@@H](C)Nc1nnc(C)c2cnc(N3CC(C)(O)C3)cc12 ; Cc1c([C@@H](C)Nc2nnc(C)c3cnc(N4C5CCC4COC5)cc23)cccc1C(F)(F)F]

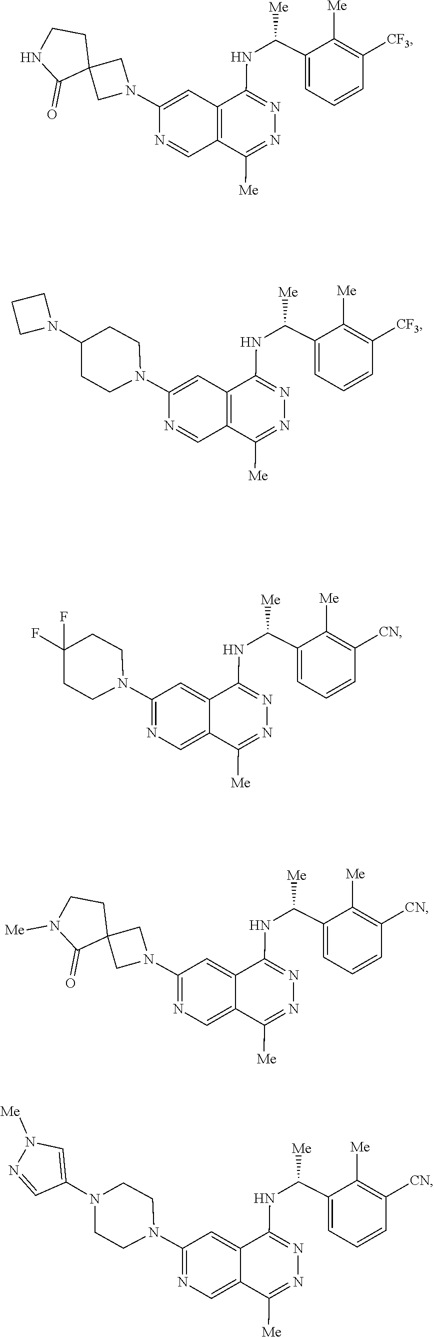 [SMILES: Cc1c([C@@H](C)Nc2nnc(C)c3cnc(N4CC5(CCNC5=O)C4)cc23)cccc1C(F)(F)F ; Cc1c([C@@H](C)Nc2nnc(C)c3cnc(N4CCC(N5CCC5)CC4)cc23)cccc1C(F)(F)F ; Cc1c(C#N)cccc1[C@@H](C)Nc1nnc(C)c2cnc(N3CCC(F)(F)CC3)cc12 ; Cc1c(C#N)cccc1[C@@H](C)Nc1nnc(C)c2cnc(N3CC4(CCN(C)C4=O)C3)cc12 ; Cc1c(C#N)cccc1[C@@H](C)Nc1nnc(C)c2cnc(N3CCN(c4cnn(C)c4)CC3)cc12]

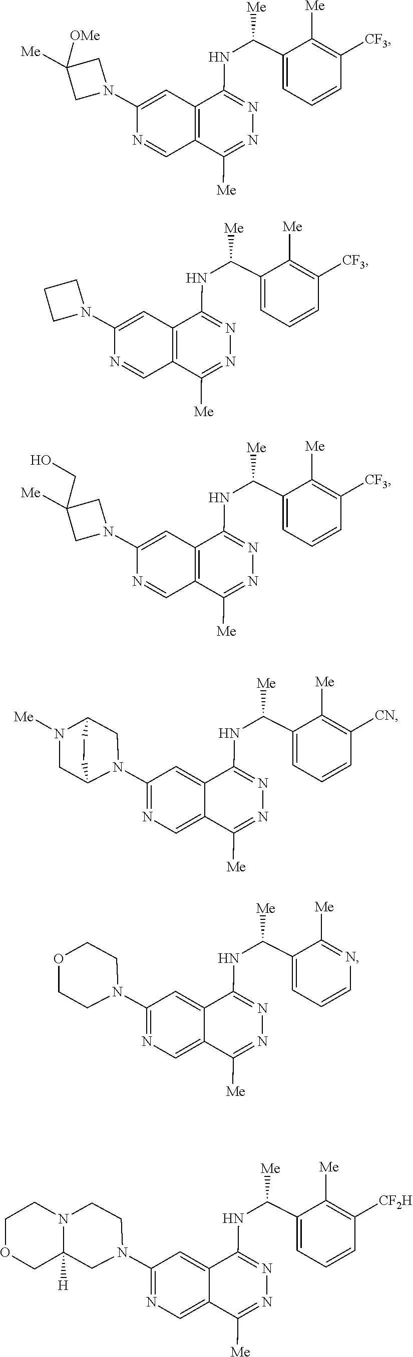

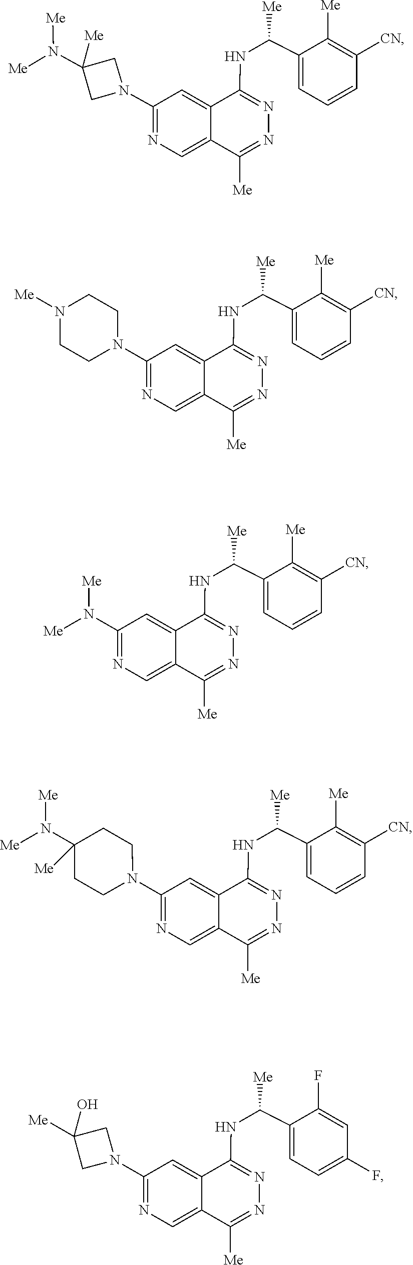 [SMILES: Cc1c(C#N)cccc1[C@@H](C)Nc1nnc(C)c2cnc(N3CC(C)(N(C)C)C3)cc12 ; Cc1c(C#N)cccc1[C@@H](C)Nc1nnc(C)c2cnc(N3CCN(C)CC3)cc12 ; Cc1c(C#N)cccc1[C@@H](C)Nc1nnc(C)c2cnc(N(C)C)cc12 ; Cc1c(C#N)cccc1[C@@H](C)Nc1nnc(C)c2cnc(N3CCC(C)(N(C)C)CC3)cc12 ; Cc1nnc(N[C@H](C)c2ccc(F)cc2F)c2cc(N3CC(C)(O)C3)ncc12]

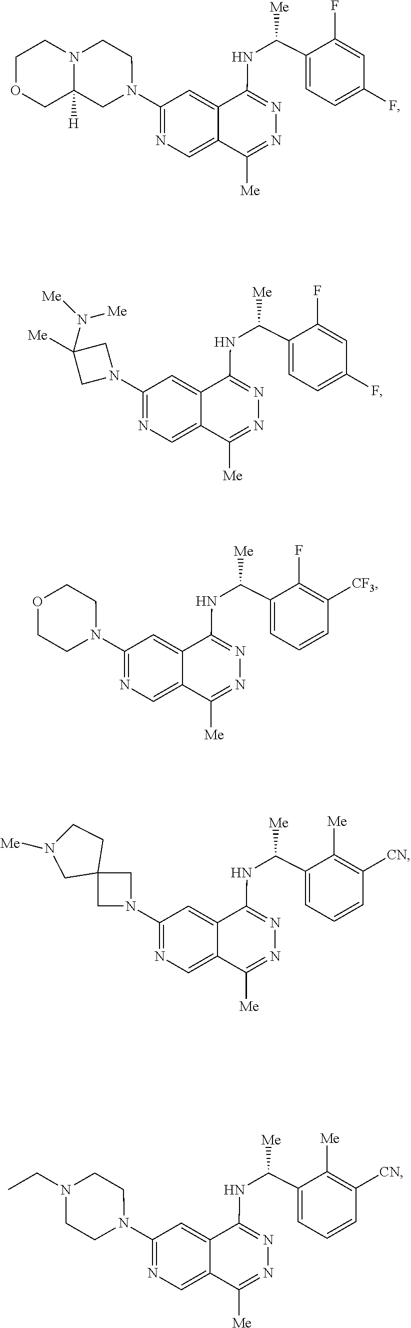 [SMILES: Cc1nnc(N[C@H](C)c2ccc(F)cc2F)c2cc(N3CCN4CCOC[C@@H]4C3)ncc12 ; Cc1nnc(N[C@H](C)c2ccc(F)cc2F)c2cc(N3CC(C)(N(C)C)C3)ncc12 ; Cc1nnc(N[C@H](C)c2cccc(C(F)(F)F)c2F)c2cc(N3CCOCC3)ncc12 ; Cc1c(C#N)cccc1[C@@H](C)Nc1nnc(C)c2cnc(N3CC4(CCN(C)C4)C3)cc12 ; CCN1CCN(c2cc3c(N[C@H](C)c4cccc(C#N)c4C)nnc(C)c3cn2)CC1]

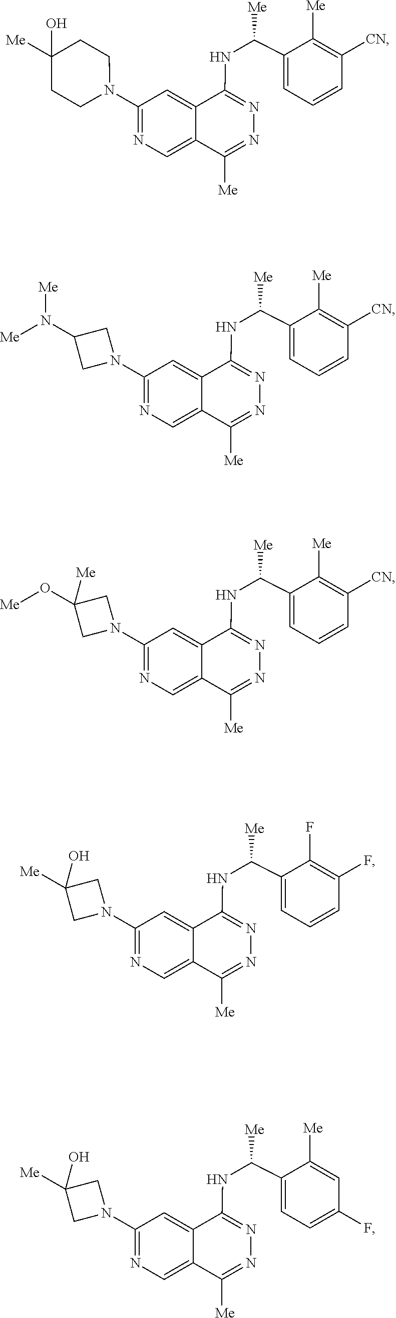 [SMILES: Cc1c(C#N)cccc1[C@@H](C)Nc1nnc(C)c2cnc(N3CCC(C)(O)CC3)cc12 ; Cc1c(C#N)cccc1[C@@H](C)Nc1nnc(C)c2cnc(N3CC(N(C)C)C3)cc12 ; COC1(C)CN(c2cc3c(N[C@H](C)c4cccc(C#N)c4C)nnc(C)c3cn2)C1 ; Cc1nnc(N[C@H](C)c2cccc(F)c2F)c2cc(N3CC(C)(O)C3)ncc12 ; Cc1cc(F)ccc1[C@@H](C)Nc1nnc(C)c2cnc(N3CC(C)(O)C3)cc12]

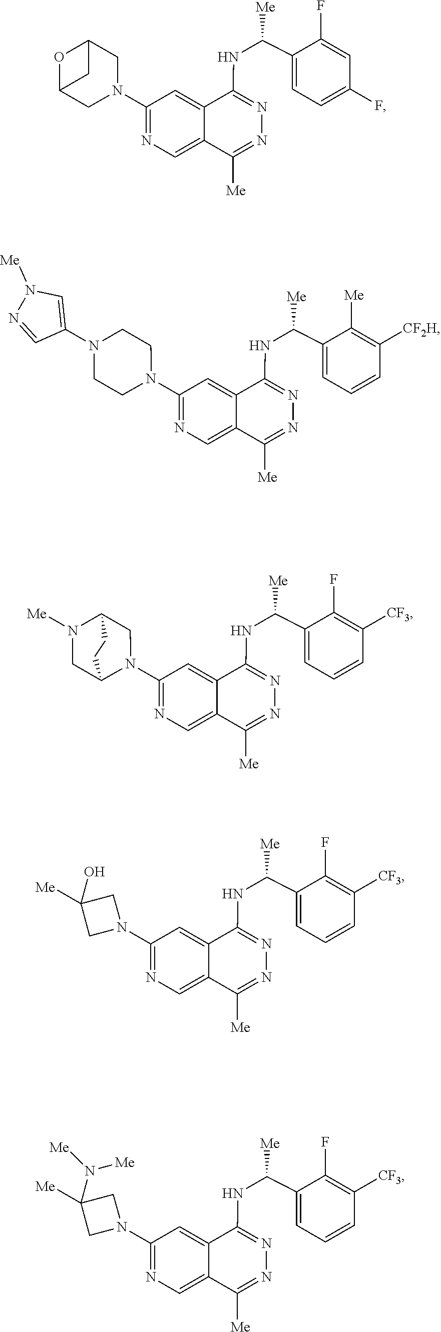 [SMILES: Cc1nnc(N[C@H](C)c2ccc(F)cc2F)c2cc(N3CC4CC(C3)O4)ncc12 ; Cc1c(C(F)F)cccc1[C@@H](C)Nc1nnc(C)c2cnc(N3CCN(c4cnn(C)c4)CC3)cc12 ; Cc1nnc(N[C@H](C)c2cccc(C(F)(F)F)c2F)c2cc(N3C[C@H]4CC[C@@H]3CN4C)ncc12 ; Cc1nnc(N[C@H](C)c2cccc(C(F)(F)F)c2F)c2cc(N3CC(C)(O)C3)ncc12 ; Cc1nnc(N[C@H](C)c2cccc(C(F)(F)F)c2F)c2cc(N3CC(C)(N(C)C)C3)ncc12]

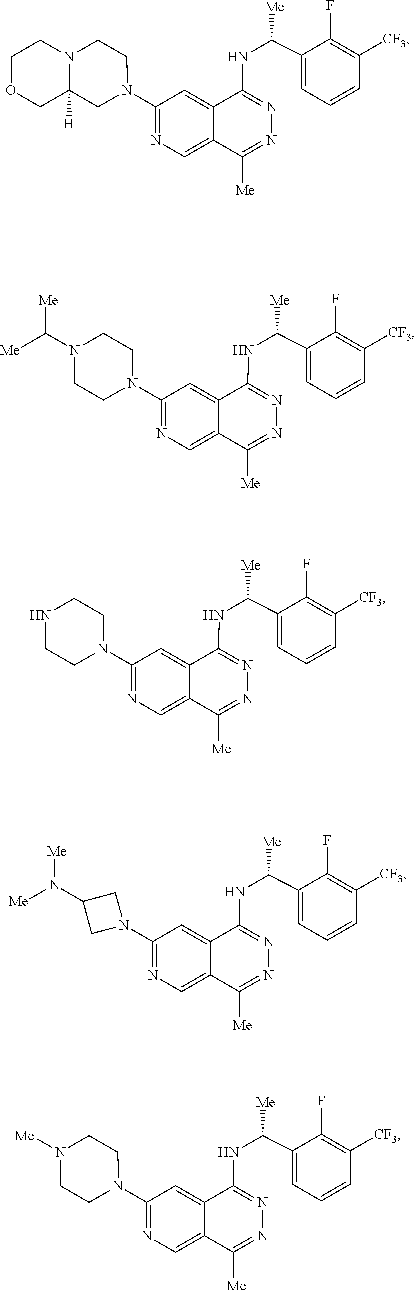 [SMILES: Cc1nnc(N[C@H](C)c2cccc(C(F)(F)F)c2F)c2cc(N3CCN4CCOC[C@@H]4C3)ncc12 ; Cc1nnc(N[C@H](C)c2cccc(C(F)(F)F)c2F)c2cc(N3CCN(C(C)C)CC3)ncc12 ; Cc1nnc(N[C@H](C)c2cccc(C(F)(F)F)c2F)c2cc(N3CCNCC3)ncc12 ; Cc1nnc(N[C@H](C)c2cccc(C(F)(F)F)c2F)c2cc(N3CC(N(C)C)C3)ncc12 ; Cc1nnc(N[C@H](C)c2cccc(C(F)(F)F)c2F)c2cc(N3CCN(C)CC3)ncc12]

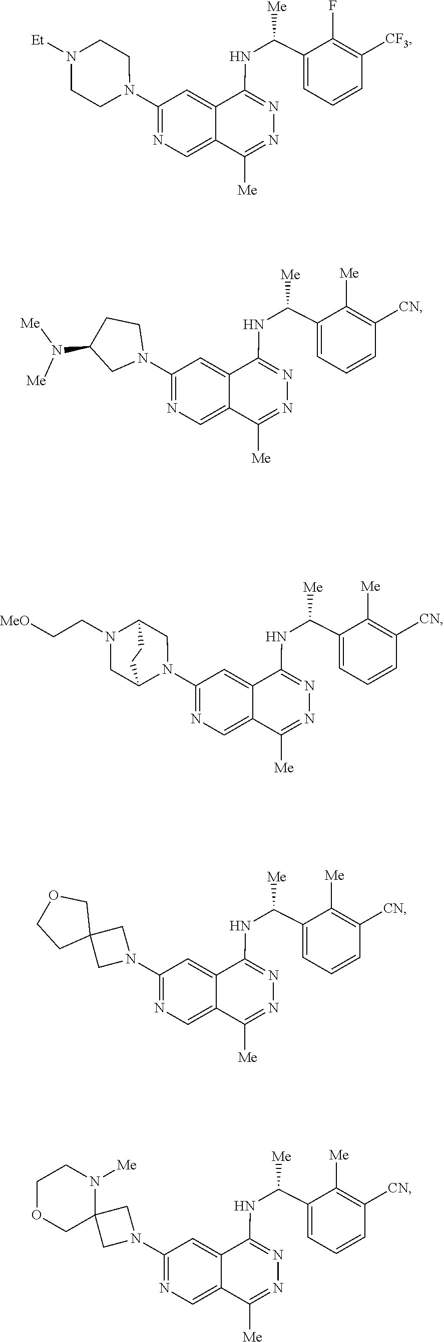 [SMILES: CCN1CCN(c2cc3c(N[C@H](C)c4cccc(C(F)(F)F)c4F)nnc(C)c3cn2)CC1 ; Cc1c(C#N)cccc1[C@@H](C)Nc1nnc(C)c2cnc(N3CC[C@H](N(C)C)C3)cc12 ; COCCN1C[C@H]2CC[C@@H]1CN2c1cc2c(N[C@H](C)c3cccc(C#N)c3C)nnc(C)c2cn1 ; Cc1c(C#N)cccc1[C@@H](C)Nc1nnc(C)c2cnc(N3CC4(CCOC4)C3)cc12 ; Cc1c(C#N)cccc1[C@@H](C)Nc1nnc(C)c2cnc(N3CC4(COCCN4C)C3)cc12]

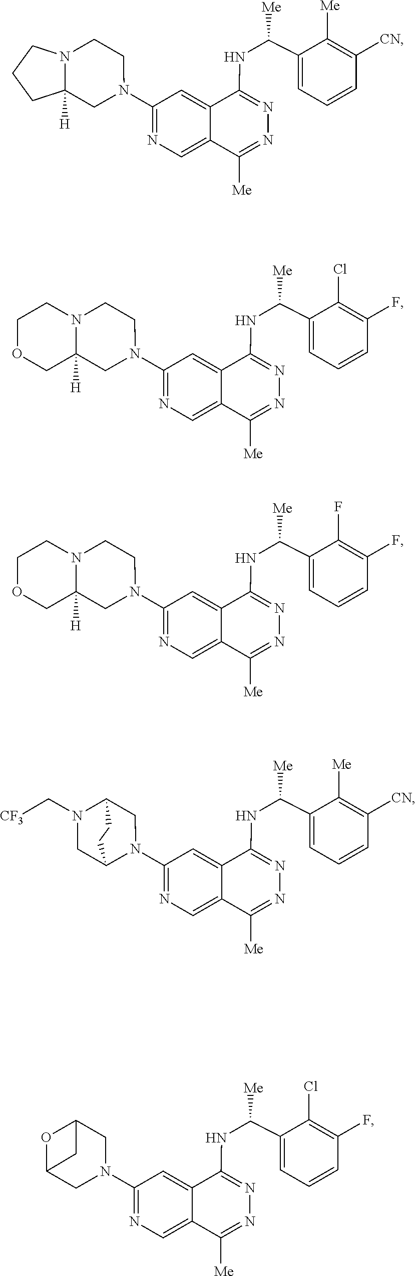 [SMILES: Cc1c(C#N)cccc1[C@@H](C)Nc1nnc(C)c2cnc(N3CCN4CCC[C@@H]4C3)cc12 ; Cc1nnc(N[C@H](C)c2cccc(F)c2Cl)c2cc(N3CCN4CCOC[C@@H]4C3)ncc12 ; Cc1nnc(N[C@H](C)c2cccc(F)c2F)c2cc(N3CCN4CCOC[C@@H]4C3)ncc12 ; Cc1c(C#N)cccc1[C@@H](C)Nc1nnc(C)c2cnc(N3C[C@H]4CC[C@@H]3CN4CC(F)(F)F)cc12 ; Cc1nnc(N[C@H](C)c2cccc(F)c2Cl)c2cc(N3CC4CC(C3)O4)ncc12]

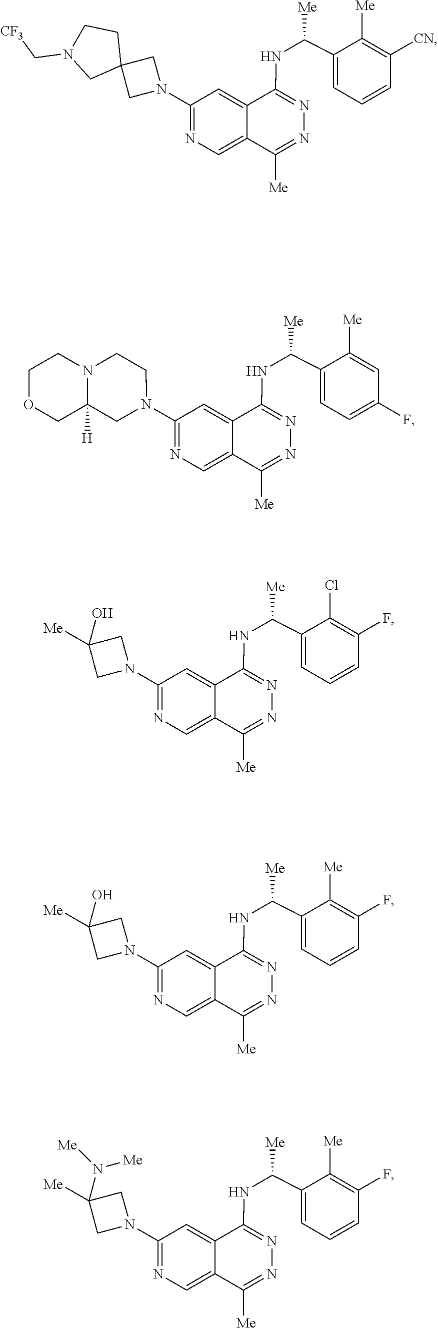 [SMILES: Cc1c(C#N)cccc1[C@@H](C)Nc1nnc(C)c2cnc(N3CC4(CCN(CC(F)(F)F)C4)C3)cc12 ; Cc1cc(F)ccc1[C@@H](C)Nc1nnc(C)c2cnc(N3CCN4CCOC[C@@H]4C3)cc12 ; Cc1nnc(N[C@H](C)c2cccc(F)c2Cl)c2cc(N3CC(C)(O)C3)ncc12 ; Cc1c(F)cccc1[C@@H](C)Nc1nnc(C)c2cnc(N3CC(C)(O)C3)cc12 ; Cc1c(F)cccc1[C@@H](C)Nc1nnc(C)c2cnc(N3CC(C)(N(C)C)C3)cc12]

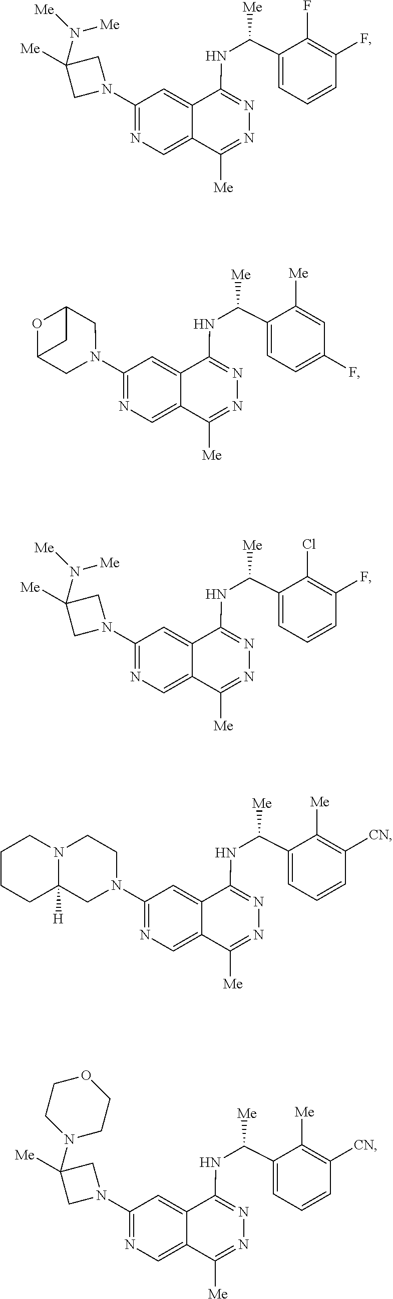 [SMILES: Cc1nnc(N[C@H](C)c2cccc(F)c2F)c2cc(N3CC(C)(N(C)C)C3)ncc12 ; Cc1cc(F)ccc1[C@@H](C)Nc1nnc(C)c2cnc(N3CC4CC(C3)O4)cc12 ; Cc1nnc(N[C@H](C)c2cccc(F)c2Cl)c2cc(N3CC(C)(N(C)C)C3)ncc12 ; Cc1c(C#N)cccc1[C@@H](C)Nc1nnc(C)c2cnc(N3CCN4CCCC[C@@H]4C3)cc12 ; Cc1c(C#N)cccc1[C@@H](C)Nc1nnc(C)c2cnc(N3CC(C)(N4CCOCC4)C3)cc12]

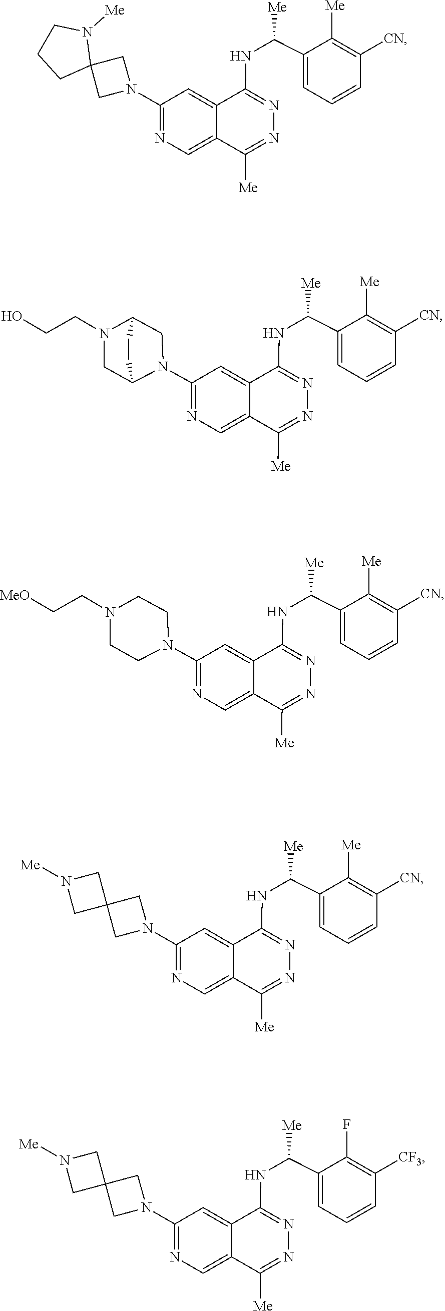 [SMILES: Cc1c(C#N)cccc1[C@@H](C)Nc1nnc(C)c2cnc(N3CC4(CCCN4C)C3)cc12 ; Cc1c(C#N)cccc1[C@@H](C)Nc1nnc(C)c2cnc(N3C[C@H]4CC[C@@H]3CN4CCO)cc12 ; COCCN1CCN(c2cc3c(N[C@H](C)c4cccc(C#N)c4C)nnc(C)c3cn2)CC1 ; Cc1c(C#N)cccc1[C@@H](C)Nc1nnc(C)c2cnc(N3CC4(CN(C)C4)C3)cc12 ; Cc1nnc(N[C@H](C)c2cccc(C(F)(F)F)c2F)c2cc(N3CC4(CN(C)C4)C3)ncc12]

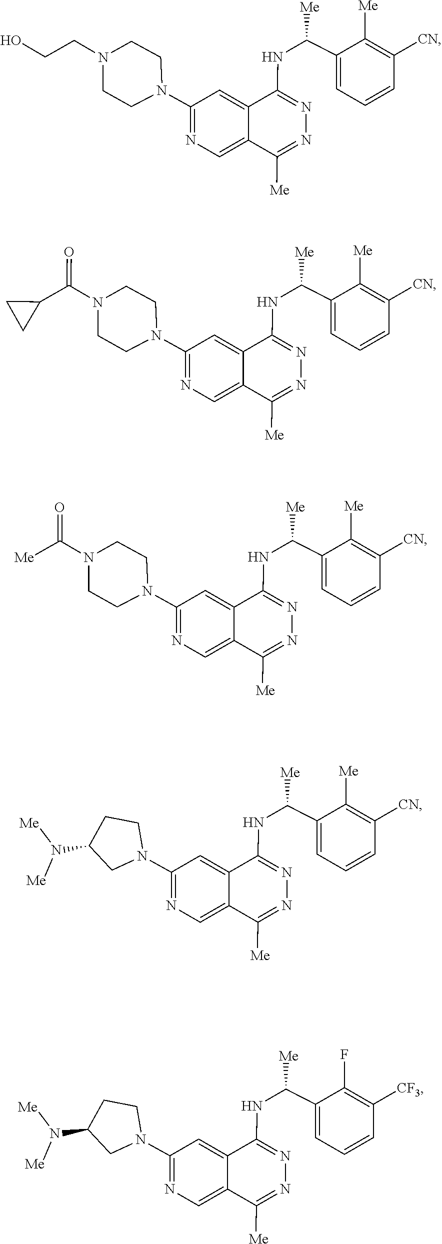 [SMILES: Cc1c(C#N)cccc1[C@@H](C)Nc1nnc(C)c2cnc(N3CCN(CCO)CC3)cc12 ; Cc1c(C#N)cccc1[C@@H](C)Nc1nnc(C)c2cnc(N3CCN(C(=O)C4CC4)CC3)cc12 ; CC(=O)N1CCN(c2cc3c(N[C@H](C)c4cccc(C#N)c4C)nnc(C)c3cn2)CC1 ; Cc1c(C#N)cccc1[C@@H](C)Nc1nnc(C)c2cnc(N3CC[C@@H](N(C)C)C3)cc12 ; Cc1nnc(N[C@H](C)c2cccc(C(F)(F)F)c2F)c2cc(N3CC[C@H](N(C)C)C3)ncc12]

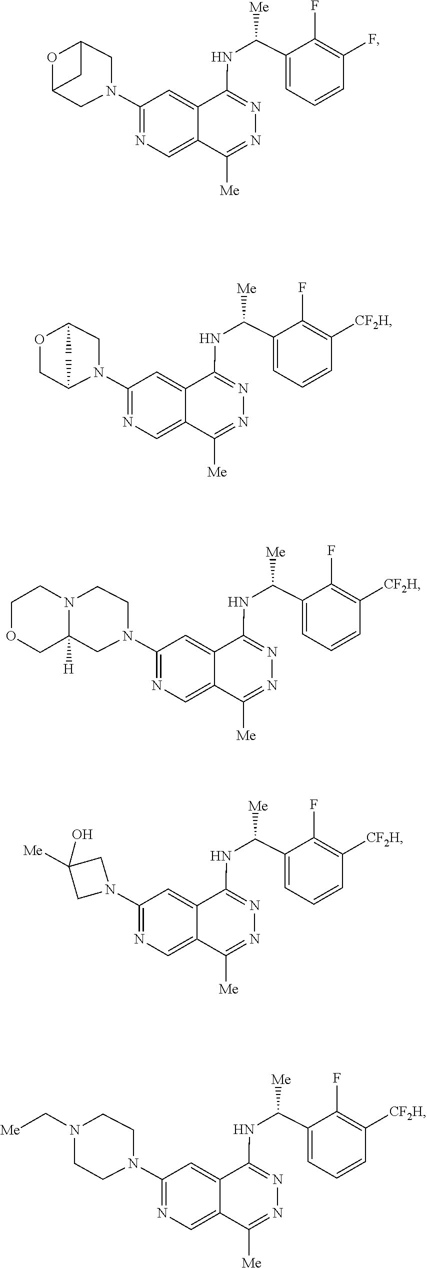 [SMILES: Cc1nnc(N[C@H](C)c2cccc(F)c2F)c2cc(N3CC4CC(C3)O4)ncc12 ; Cc1nnc(N[C@H](C)c2cccc(C(F)F)c2F)c2cc(N3C[C@H]4C[C@@H]3CO4)ncc12 ; Cc1nnc(N[C@H](C)c2cccc(C(F)F)c2F)c2cc(N3CCN4CCOC[C@@H]4C3)ncc12 ; Cc1nnc(N[C@H](C)c2cccc(C(F)F)c2F)c2cc(N3CC(C)(O)C3)ncc12 ; CCN1CCN(c2cc3c(N[C@H](C)c4cccc(C(F)F)c4F)nnc(C)c3cn2)CC1]

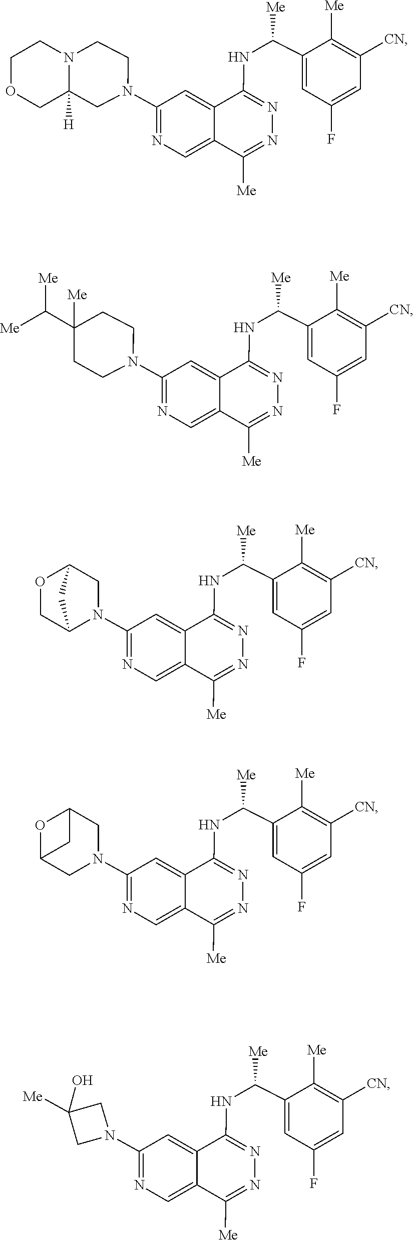 [SMILES: Cc1c(C#N)cc(F)cc1[C@@H](C)Nc1nnc(C)c2cnc(N3CCN4CCOC[C@@H]4C3)cc12 ; Cc1c(C#N)cc(F)cc1[C@@H](C)Nc1nnc(C)c2cnc(N3CCC(C)(C(C)C)CC3)cc12 ; Cc1c(C#N)cc(F)cc1[C@@H](C)Nc1nnc(C)c2cnc(N3C[C@H]4C[C@@H]3CO4)cc12 ; Cc1c(C#N)cc(F)cc1[C@@H](C)Nc1nnc(C)c2cnc(N3CC4CC(C3)O4)cc12 ; Cc1c(C#N)cc(F)cc1[C@@H](C)Nc1nnc(C)c2cnc(N3CC(C)(O)C3)cc12]

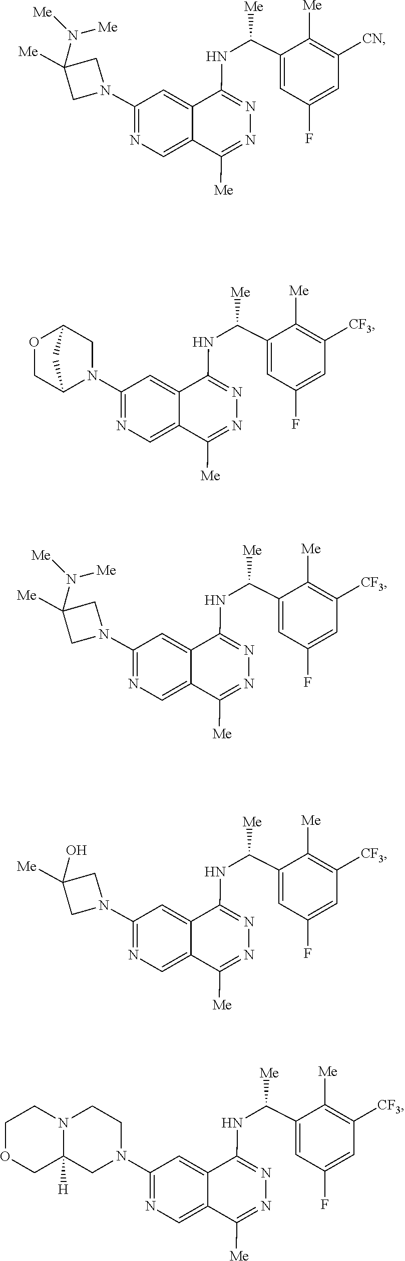 [SMILES: Cc1c(C#N)cc(F)cc1[C@@H](C)Nc1nnc(C)c2cnc(N3CC(C)(N(C)C)C3)cc12 ; Cc1c([C@@H](C)Nc2nnc(C)c3cnc(N4C[C@H]5C[C@@H]4CO5)cc23)cc(F)cc1C(F)(F)F ; Cc1c([C@@H](C)Nc2nnc(C)c3cnc(N4CC(C)(N(C)C)C4)cc23)cc(F)cc1C(F)(F)F ; Cc1c([C@@H](C)Nc2nnc(C)c3cnc(N4CC(C)(O)C4)cc23)cc(F)cc1C(F)(F)F ; Cc1c([C@@H](C)Nc2nnc(C)c3cnc(N4CCN5CCOC[C@@H]5C4)cc23)cc(F)cc1C(F)(F)F]

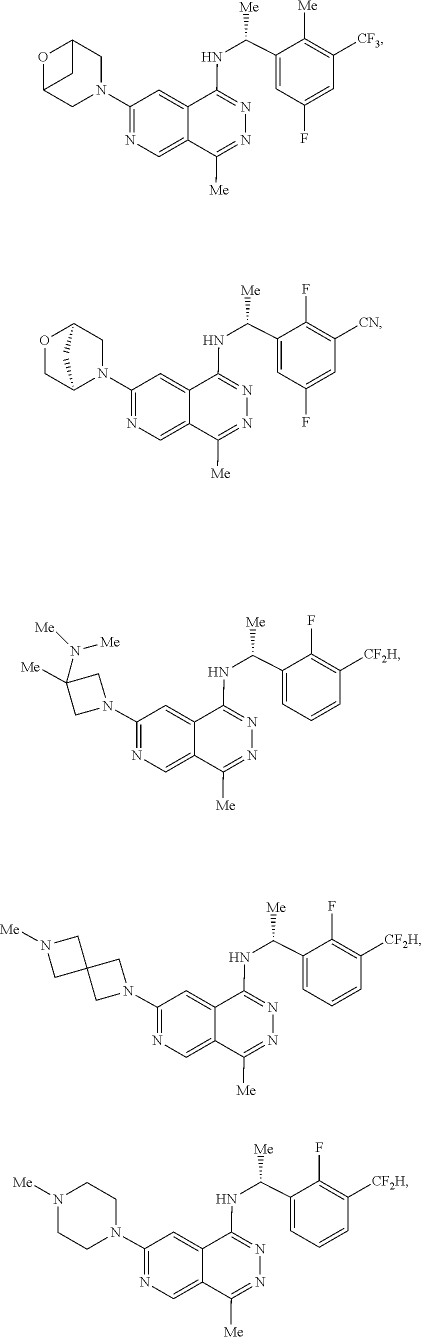 [SMILES: Cc1c([C@@H](C)Nc2nnc(C)c3cnc(N4CC5CC(C4)O5)cc23)cc(F)cc1C(F)(F)F ; Cc1nnc(N[C@H](C)c2cc(F)cc(C#N)c2F)c2cc(N3C[C@H]4C[C@@H]3CO4)ncc12 ; Cc1nnc(N[C@H](C)c2cccc(C(F)F)c2F)c2cc(N3CC(C)(N(C)C)C3)ncc12 ; Cc1nnc(N[C@H](C)c2cccc(C(F)F)c2F)c2cc(N3CC4(CN(C)C4)C3)ncc12 ; Cc1nnc(N[C@H](C)c2cccc(C(F)F)c2F)c2cc(N3CCN(C)CC3)ncc12]

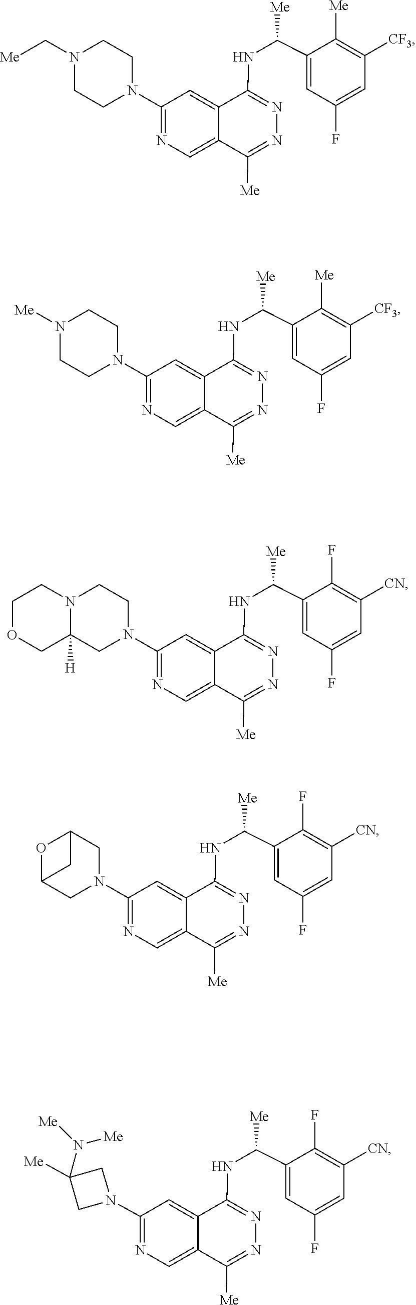 [SMILES: CCN1CCN(c2cc3c(N[C@H](C)c4cc(F)cc(C(F)(F)F)c4C)nnc(C)c3cn2)CC1 ; Cc1c([C@@H](C)Nc2nnc(C)c3cnc(N4CCN(C)CC4)cc23)cc(F)cc1C(F)(F)F ; Cc1nnc(N[C@H](C)c2cc(F)cc(C#N)c2F)c2cc(N3CCN4CCOC[C@@H]4C3)ncc12 ; Cc1nnc(N[C@H](C)c2cc(F)cc(C#N)c2F)c2cc(N3CC4CC(C3)O4)ncc12 ; Cc1nnc(N[C@H](C)c2cc(F)cc(C#N)c2F)c2cc(N3CC(C)(N(C)C)C3)ncc12]

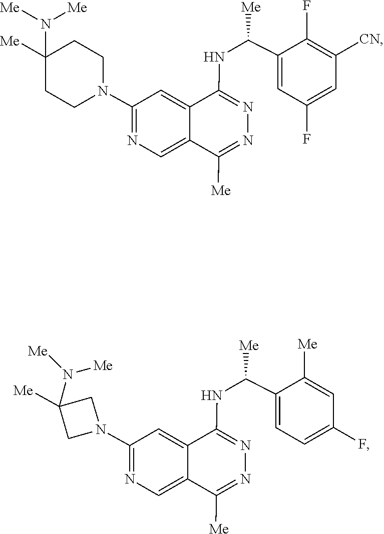 [SMILES: Cc1nnc(N[C@H](C)c2cc(F)cc(C#N)c2F)c2cc(N3CCC(C)(N(C)C)CC3)ncc12 ; Cc1cc(F)ccc1[C@@H](C)Nc1nnc(C)c2cnc(N3CC(C)(N(C)C)C3)cc12]

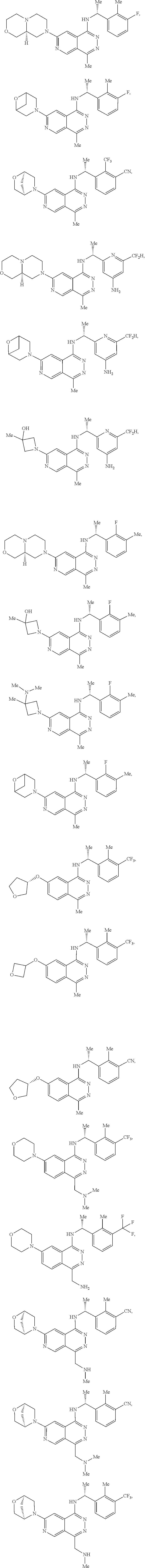

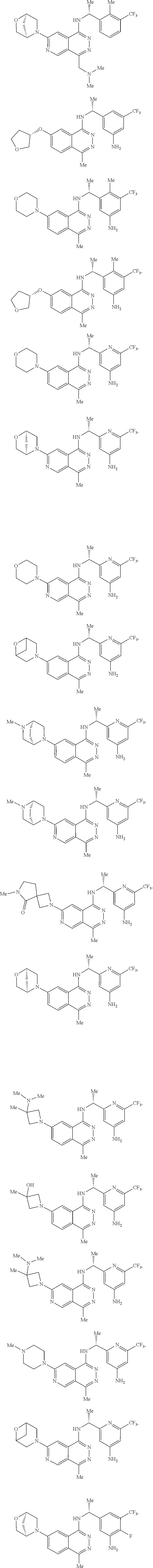

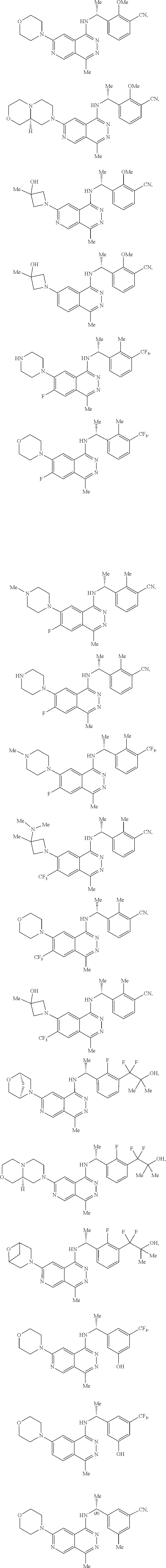

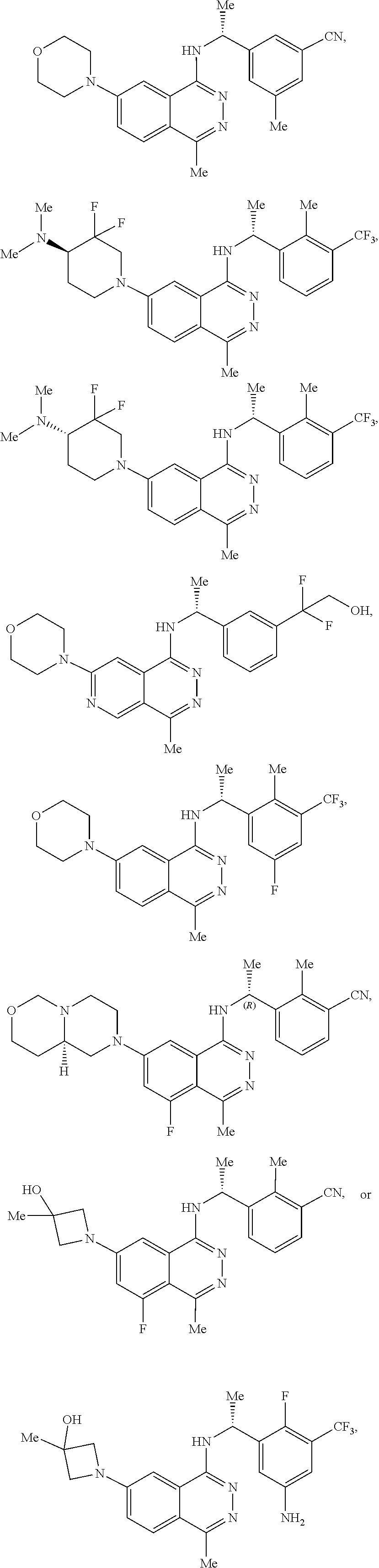

and pharmaceutically acceptable salts of the foregoing compounds.

In certain embodiment of the method of the disclosure as described herein, the SOS1 inhibitor is

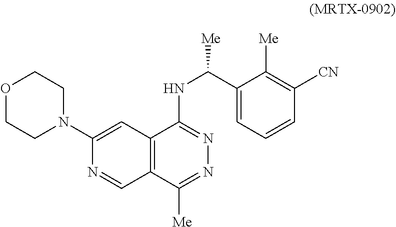 [SMILES: Cc1c(C#N)cccc1[C@@H](C)Nc1nnc(C)c2cnc(N3CCOCC3)cc12]

or a pharmaceutical salt thereof.

### PRMT5 Inhibitors

As provided above, the PRMT5 inhibitor is also administered in the methods of the disclosure. A “PRMT5 inhibitor” as used herein refers to compounds of the disclosure as described herein. These compounds are capable of negatively modulating or inhibiting all or a portion of the enzymatic activity of the PRMT5, particularly, in the presence of bound MTA in vitro or in vivo or in cells expressing elevated levels of MTA. In certain embodiments, the PRMT5 inhibitor is a MTA-cooperative PRMT5 inhibitor.

In certain embodiments, the PRMT5 inhibitor of the disclosure is any one of the PRMT5 inhibitors disclosed in International patent publication No. WO 2021/050915 A1, published 18 Mar. 2021, incorporated by reference in its entirety.

In certain other embodiments, the PRMT5 inhibitor of the disclosure is any one of the PRMT5 inhibitors disclosed in International patent publication No. WO 2022/192745, published 15 Sep. 2022, incorporated by reference in its entirety.

In certain other embodiments, the PRMT5 inhibitor of the disclosure is any one of the PRMT5 inhibitors disclosed in International patent publication No. WO2023/081367, published 3 Aug. 2023, incorporated by reference in its entirety.

In certain other embodiments, the PRMT 5 inhibitor of the disclosure is any one of the PRMT5 inhibitors disclosed in International patent publication No. WO2023/278564, published 5 Jan. 2023, incorporated by reference in its entirety.

For example, the PRMT5 inhibitor in the methods of the disclosure as described herein is a compound of Formula IIA, IIB or IIC 1:

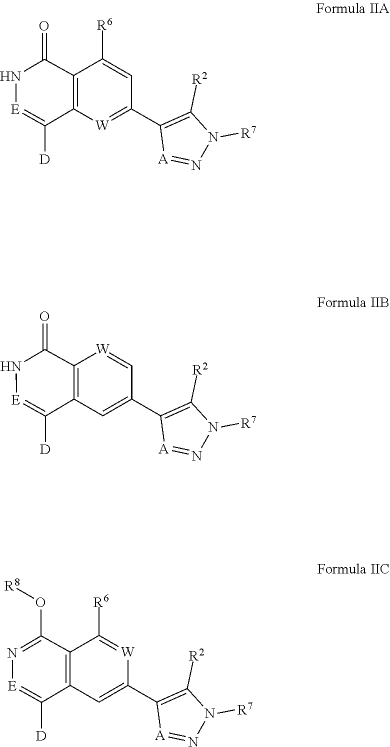

or a pharmaceutically acceptable salt thereof, wherein:
A is CR⁹ or N;
D is (C(R⁹)₂)₁₋₂—NH₂,

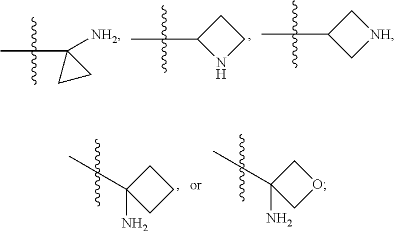 [SMILES: *C1(N)CC1 ; *C1CCN1 ; *C1CNC1 ; *C1(N)CCC1 ; *C1(N)COC1]

### or D is

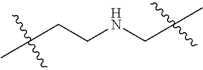 [SMILES: *CCNC*]

where the methylene is bonded to E where E is C;
E is C, CR⁹ or N;
each L is independently a bond or C₁-C₃ alkylene;
W is CR⁹ or N;
each X is independently a bond, O, S, —NR⁴— or —NR⁴C(O)—;
each Z is independently a bond, —SO—, —SO₂—, —CH(OH)— or —C(O)—;
each R² is independently hydroxy, halogen, cyano, cyanomethyl, —(NR⁴)₂, hydroxyalkyl, alkoxy, —SO₂C₁-C₃alkyl, —X-arC₁-C₃alkyl, heteroalkyl, C₂-C₄ alkynyl, —X-haloalkyl, —X—C₁-C₅ alkyl, —Z—C₁-C₅ alkyl, heterocyclyl, —X-L-cycloalkyl, —Z-cycloalkyl, —X-aryl, —Z-aryl, or —X— heteroaryl, wherein the heterocyclyl, the cycloalkyl, the aryl and the heteroaryl are optionally substituted with one or more R⁵;
each R⁴ is independently hydrogen or C₁-C₃ alkyl;
each R⁵ is independently cyano, oxo, halogen, C₁-C₃ alkyl, hydroxyalkyl, hydroxy, alkoxy, alkoxy-C₁-C₃ alkyl, —X-haloalkyl, —Z-cycloalkyl, —X-arC₁-C₃alkyl, —X-arC₁-C₃alkyl substituted with cyano, —X-L-cycloalkyl optionally substituted with C₁-C₃ alkyl or oxo, —X-L-heteroaryl optionally substituted with one or more C₁-C₃ alkyl or oxo, —X-L-heterocyclyl optionally substituted with one or more C₁-C₃ alkyl or oxo, or —X-aryl;
R⁶ is hydrogen, halogen, C₁-C₃ alkyl, haloalkyl, hydroxy, alkoxy, C₁-C₃ alkyl-alkoxy, N(R⁹)₂, NR⁹C(O)R⁹, C(O)R⁹, oxetane and THF;
R⁷ is H or C₁-C₃ alkyl optionally substituted with one or more halogen;
R⁸ is H or C₁-C₃ alkyl; and
each R⁹ is independently H or C₁-C₃ alkyl, halogen or haloalkyl.

Embodiment 2 provides the PRMT5 inhibitor in the methods of the disclosure as a compound of Formula IIA:

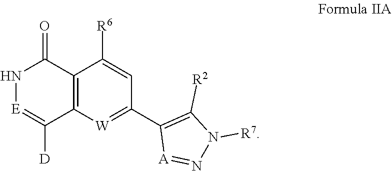

In certain embodiments, the PRMT5 inhibitor in the methods of the disclosure as a compound of Formula IIB:

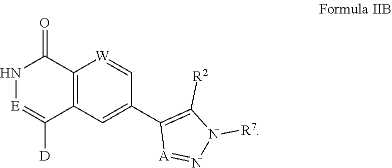

In certain embodiments, the PRMT5 inhibitor in the methods of the disclosure as a compound of Formula IIC:

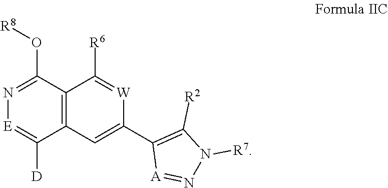

In certain embodiments of Formula IIA, IIB, and IIC, W is CR⁹.

In certain embodiments of Formula IIA, IIB, and IIC, A is CR⁹.

In certain embodiments of Formula IIA, IIB, and IIC, E is N.

In certain embodiments of Formula IIA, IIB, and IIC, W is CR⁹, A is CR⁹ and E is N.

Embodiment 9 provides the method of any of embodiments 1-8, wherein R² is selected from: benzothiophene, naphthalene, quinoline, chromane, isochromane, dihydrobenzodioxine, indolazine, tetrahydroindolazine, dihydroisobenzofuran, benzene, isoquinolinone, benzodioxone, thienopyridine, tetrahydroindolone, indolizine, dihydroindolizinone, imadazopyridinone, thienopyrimidine, thiophene, pyrrolopyrimidinone, thiazolopyridinone, dihydropyrrolizine, isoindalone and tetrahydroisoquinoline.

In certain embodiments of Formula IIA, IIB, and IIC, each R⁵ is independently cyano, oxo, halogen, C1-C3 alkyl, hydroxy, hydroxyalkyl, alkoxy-C1-C3alkyl, —X-L-heterocyclyl optionally substituted with one or more C1-C3alkyl or oxo, —X-L-cycloalkyl optionally substituted with C1-C3 alkyl or oxo.

In certain embodiments of Formula IIA, IIB, and IIC, R⁶ is selected from hydrogen, hydroxy, chlorine, —NHC(O)CH₃, —C(O)CF₂H, —NH₂, —CF₂, —CH₃, —O—CH₂CH₃, —CH₂—CH₂—O—CH₃, oxetane and THF.

In certain embodiments of Formula IIA, IIB, and IIC, one of L, X and Z is a bond. In certain embodiments, all of L, X and Z are bonds.

One aspect of the disclosure provides the method wherein the PRMT5 inhibitor is a compound of the Formula IIIC:

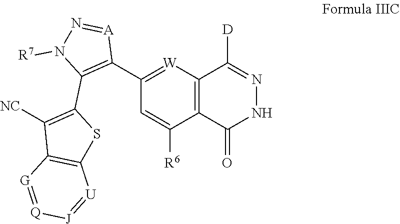

or a pharmaceutically acceptable salt thereof, wherein
A is CR⁹ or N;
D is —CH₂—NH₂,

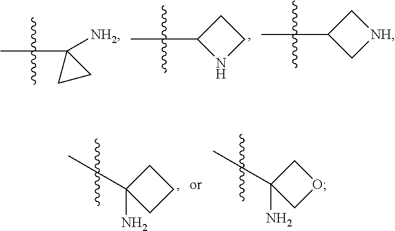 [SMILES: *C1(N)CC1 ; *C1CCN1 ; *C1CNC1 ; *C1(N)CCC1 ; *C1(N)COC1]

W is CR⁹ or N, where R⁹ is H or C₁-C₃ alkyl;
G, Q, J and U are independently selected from C(H), C(R⁵), and N, provided only one or two of G, Q, J, and U can be N;
   each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl;
R⁶ is hydrogen, halogen, C₁-C₆ alkyl, C₁-C₆ haloalkyl, hydroxy, C₁-C₆ alkoxy, C₁-C₃ alkoxyC₁-C₃ alkyl, C₃-C₆ heterocycloalkyl, —C(O)—C₁-C₃ haloalkyl, —N(R⁹)₂,
   or —NR¹⁵(CO)R¹⁶, where each R⁹ is independently H or C₁-C₃ alkyl, R¹⁵ is hydrogen or methyl, and R¹⁶ is C₁-C₃ alkyl; and
R⁷ is C₁-C₃ alkyl or C₁-C₃ haloalkyl.

In certain embodiments of Formula IIIC, A is CH.

In certain embodiments of Formula IIIC, W is N.

In certain embodiments of Formula IIIC, W is CH.

In certain embodiments of Formula IIIC, D is —CH₂—NH₂.

In certain embodiments, the PRMT5 inhibitor is a compound according Formula IIIC having the formula:

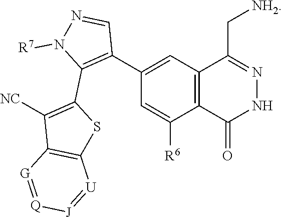

In certain embodiments of Formula IIIC, R⁶ is hydrogen, halogen, C₁-C₆ alkyl, C₁-C₆ haloalkyl, hydroxy, C₁-C₆ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

In certain embodiments of Formula IIIC, R⁶ is hydrogen, halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

In certain embodiments of Formula IIIC, R⁶ is hydrogen, chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

In certain embodiments of Formula IIIC, R⁶ is halogen, C₁-C₆ alkyl, C₁-C₆ haloalkyl, hydroxy, C₁-C₆ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

In certain embodiments of Formula IIIC, R⁶ is halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

In certain embodiments of Formula IIIC, R⁶ is chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

In certain embodiments of Formula IIIC, each G, Q, J and U is independently C(H).

In certain embodiments of Formula IIIC, G, Q, J and U are independently selected from C(H) and C(R⁵).

In certain embodiments of Formula IIIC, G, Q, J and U are independently selected from C(H) and N.

In certain embodiments of Formula IIIC,
R⁶ is hydrogen;
at least one of G, Q, J, and U is C(R⁵), and the remaining G, Q, J, and U are independently selected from C(H), C(R⁵) and N, wherein each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl.

For example, in certain embodiments, one or two of G, Q, J and U is N.

In certain embodiments of Formula IIIC,
R⁶ is hydrogen;
at least one of G, Q, J, and U is C(R⁵), and the remaining G, Q, J, and U are independently selected from C(H) and C(R⁵), wherein each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl.

For example, in certain embodiments, at least one of G, Q, J, and U is C(R⁵), and the remaining G, Q, J, and U are independently C(H); for example only one of G, Q, J, and U is C(R⁵). In certain embodiments, two of G, Q, J, and U is C(R⁵), and the remaining G, Q, J, and U are independently C(H). In certain embodiments, three of G, Q, J, and U is C(R⁵), and the remaining G, Q, J, and U is C(H).

In certain embodiments of Formula IIIC, G, Q, J, and U together with the thiophene to which they are attached form:

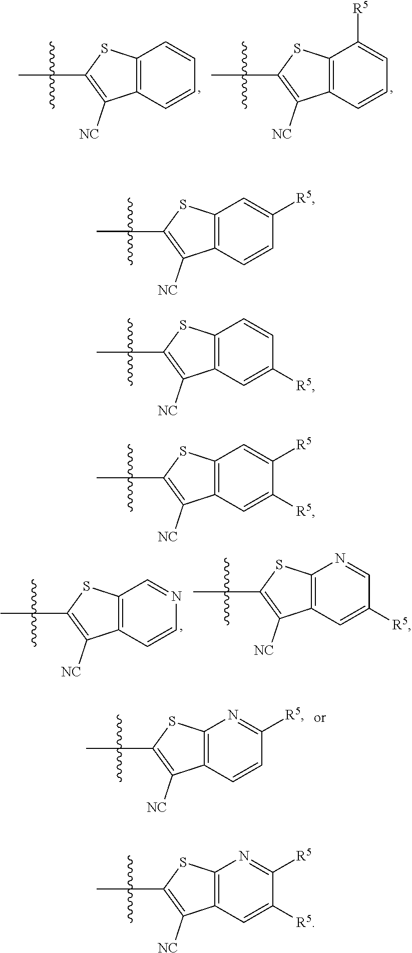

In certain embodiments of Formula IIIC, G, Q, J, and U together with the thiophene ring to which they are attached form a benzo[b]thiophene.

In certain embodiments of Formula IIIC, R⁵, if present, is hydroxy, halogen, C₁-C₃ alkyl, C₁-C₃ haloalkyl, C₁-C₃ alkoxy, C₁-C₃ haloalkoxy, C₃-C₆ cycloalkoxy, C₃-C₆ cycloalkyl, C₃-C₆ heterocycloalkyl, or C₁-C₃ alkoxyC₁-C₃ alkyl.

In certain embodiments of Formula IIIC, R⁵, if present, is hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl.

In certain embodiments of Formula IIIC, R⁵, if present, is hydroxy, chloro, fluoro, methyl, ethyl, methoxy, ethoxy, 2,2-difluoroethoxy, oxetanyl, tetrahydrofuranyl, (methoxy)methyl, (ethoxy)methyl, (methoxy)ethyl, or (ethoxy)ethyl.

In certain embodiments of Formula IIIC, R⁷ is methyl.

In certain embodiments of Formula IIIC, R⁷ is ethyl.

In certain embodiments of Formula IIIC, R⁷ is propyl (e.g., isopropyl).

In certain embodiments of Formula IIIC, R⁷ is difluoromethyl or trifluoromethyl.

In certain embodiments of Formula IIIC, the PRMT5 inhibitor is of the formula:

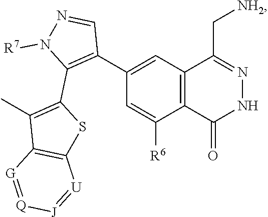

wherein
G, Q, J, and U together with the thiophene to which they are attached form:

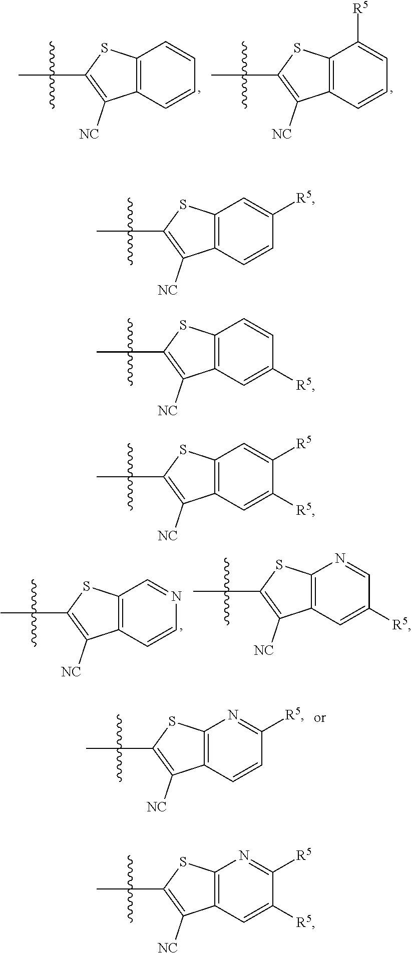

where each R⁵ is independently hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl; and
R⁶ is hydrogen, halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

In certain embodiments of Formula IIIC, the PRMT5 inhibitor is of the formula:

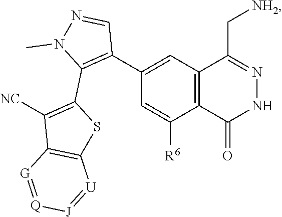

wherein
G, Q, J, and U together with the thiophene to which they are attached form:

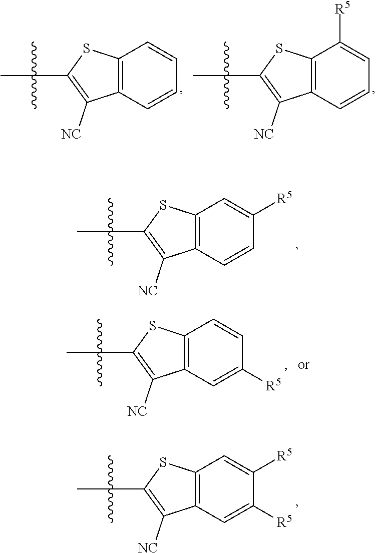

where each R⁵ is independently hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl; and
R⁶ is halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶

In certain embodiments of Formula IIIC, the PRMT5 inhibitor is of the formula:

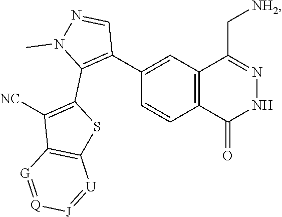

wherein
G, Q, J, and U together with the thiophene to which they are attached form:

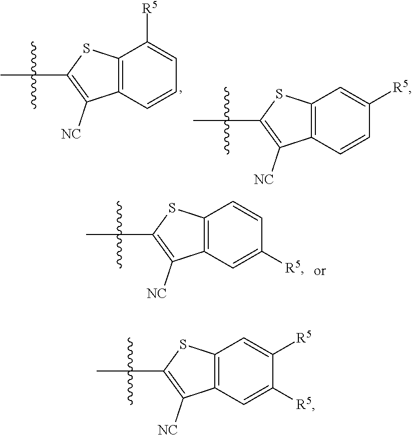

where each R⁵ is independently hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl.

In certain embodiments of the methods as described herein, the PRMT5 inhibitor is: the PRMT5 inhibitor is:

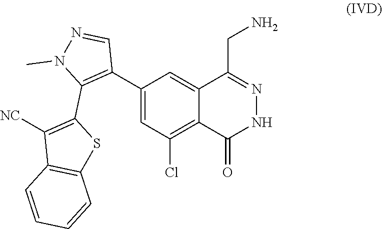 [SMILES: Cn1ncc(-c2cc(Cl)c3c(=O)[nH]nc(CN)c3c2)c1-c1sc2ccccc2c1C#N]

or a pharmaceutically acceptable salt thereof.

In certain embodiments, the PRMT5 inhibitor is a compound of the Formula IIIB:

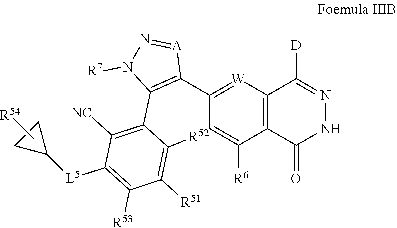

or a pharmaceutically acceptable salt thereof, wherein
A is CR⁹ or N;
D is —CH₂—NH₂,

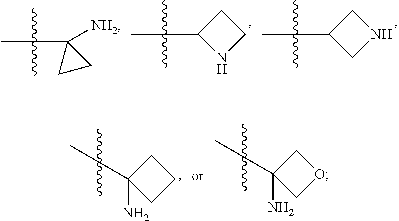

W is CR⁹ or N, where R⁹ is H or C₁-C₃ alkyl;
R⁵¹ is hydrogen, fluoro, chloro, or methyl, or R¹¹ and R⁵² together with atoms to which they are attached form a C₄-C₆ heterocycloalkyl (e.g, hydrofuranyl);
R⁵² is fluoro, chloro, or methyl, or R⁵² and R⁵³ together with atoms to which they are attached form a phenyl;
R⁵³ is hydrogen, fluoro, chloro, or methyl;
R⁵⁴ is hydrogen, halogen, C₁-C₃ alkyl, or C₁-C₃ alkoxy; L⁵ is —O— or —CH₂—;
R⁶ is hydrogen, halogen, C₁-C₆ alkyl, hydroxy, C₁-C₆ alkoxy, C₁-C₃ alkoxyC₁-C₃ alkyl, C₃-C₆ heterocycloalkyl, —C(O)—C₁-C₃ haloalkyl, or —NR¹⁵(CO)R¹⁶, where R¹⁵ is hydrogen or methyl, and R¹⁶ is C₁-C₃ alkyl;
R⁷ is C₁-C₃ alkyl or C₁-C₃ haloalkyl.

In certain embodiments of Formula IIIB:
A is —CH or —CCH₃;
D is —CH₂—NH₂;
W is —CH, —CCH₃, or N;
R⁵¹, R⁵², R⁵³, and R⁵⁴ are each independently selected from hydrogen, fluoro, chloro, or methyl;
L⁵ is —O—;
R⁶ is hydrogen, fluoro, chloro, or methyl; and
R⁷ is C₁-C₂ alkyl or C₁-C₂ haloalkyl.

In certain embodiments of Formula IIIB:
A and W are —CH;
D is —CH₂—NH₂;
R⁵¹, R⁵², and R⁵³ are each independently selected from hydrogen, fluoro, chloro, and methyl;
R⁵⁴ is hydrogen;
L⁵ is —O—;
R⁶ is hydrogen; and
R⁷ is methyl.

In certain embodiments of Formula IIIB:
A and W are —CH;
D is —CH₂—NH₂;
R⁵¹ and R⁵² are each independently selected from fluoro, chloro, and methyl;
R⁵³ and R⁵⁴ are hydrogen;
L⁵ is —O—;
R⁶ is hydrogen; and
R⁷ is methyl.

In certain embodiments of Formula IIIB, A is CH.

In certain embodiments of Formula IIIB, W is N.

In certain embodiments of Formula IIIB, W is CH.

In certain embodiments of Formula IIIB, D is —CH₂—NH₂.

In certain embodiments of Formula IIIB, R⁵⁴ is hydrogen or methyl.

In certain embodiments of Formula IIIB, R⁵⁴ is hydrogen.

In certain embodiments of Formula IIIB, R⁵⁴ is methyl.

In certain embodiments of Formula IIIB, the PRMT5 inhibitor is of the formula:

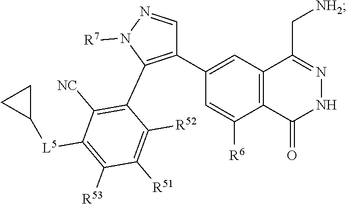

such as e.g.,

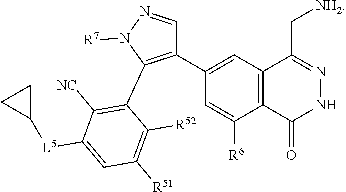

In certain embodiments of Formula IIIB, L⁵ is —CH₂—.

In certain embodiments of Formula IIIB, L⁵ is —O—.

In certain embodiments of Formula IIIB, R⁶ is hydrogen, halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶; for example, wherein R⁶ is hydrogen, chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)— difluoromethyl, —NH₂, or —NH(CO)CH₃.

In certain embodiments of Formula IIIB, R⁶ is hydrogen, halogen, C₁-C₆ alkyl, or C₁-C₆ alkoxy; for example, R⁶ is hydrogen, halogen, C₁-C₃ alkyl, or C₁-C₃ alkoxy.

In certain embodiments of Formula IIIB, R⁶ is hydrogen, chloro, fluoro, methyl, ethyl, methoxy, or ethoxy.

In certain embodiments of Formula IIIB, R⁶ is halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶; for example, wherein R⁶ is chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

In certain embodiments of Formula IIIB, R⁶ is halogen, C₁-C₆ alkyl, or C₁-C₆ alkoxy; for example, R⁶ is halogen, C₁-C₃ alkyl, or C₁-C₃ alkoxy.

In certain embodiments of Formula IIIB, R⁶ is chloro, fluoro, methyl, ethyl, methoxy, or ethoxy.

In certain embodiments of Formula IIIB, R⁷ is methyl.

In certain embodiments of Formula IIIB, R⁷ is ethyl.

In certain embodiments of Formula IIIB, R⁷ is propyl (e.g., isopropyl).

In certain embodiments of Formula IIIB, R⁷ is difluoromethyl or trifluoromethyl.

In certain embodiments of Formula IIIB, R⁵³ is hydrogen or methoxy; or wherein R¹³ is hydrogen.

In certain embodiments of Formula IIIB, the PRMT5 inhibitor is of the formula:

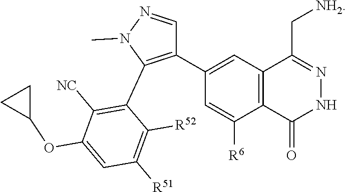

In certain embodiments, R⁵² is fluoro, and R⁵¹ is hydrogen, fluoro, chloro, or methyl.

In certain embodiments of Formula IIIB, R⁵² is fluoro, and R⁵¹ is chloro.

In certain embodiments of Formula IIIB, R⁵² is fluoro, and R⁵¹ is methyl or hydrogen (for example, R⁵² is fluoro and R¹¹ is methyl; or R⁵² is fluoro and R⁵¹ is hydrogen).

In certain embodiments of Formula IIIB, R⁵¹ and R⁵² together with atoms to which they are attached form a hydrofuranyl

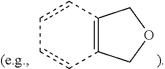

In certain embodiments of Formula IIIB, the PRMT5 inhibitor is

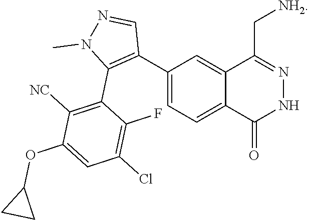 [SMILES: Cn1ncc(-c2ccc3c(=O)[nH]nc(CN)c3c2)c1-c1c(F)c(Cl)cc(OC2CC2)c1C#N]

In certain embodiments of Formula IIIB, the PRMT5 inhibitor is

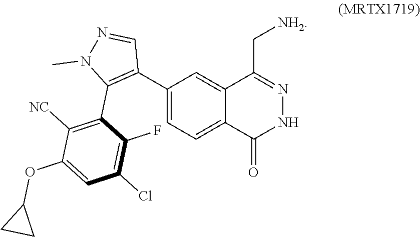

One aspect of the disclosure provides the method wherein the PRMT5 inhibitor is a compound of the Formula IIIA:

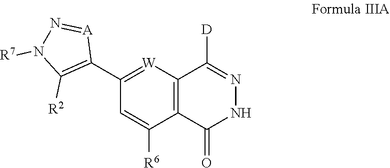

or a pharmaceutically acceptable salt thereof, wherein
A is CR⁹ or N;
D is —CH₂—NH₂,

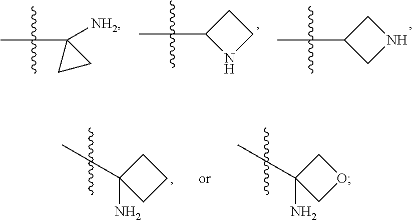 [SMILES: *C1(N)CC1 ; *C1CCN1 ; *C1CNC1 ; *C1(N)CCC1 ; *C1(N)COC1]

W is CR⁹ or N, where R⁹ is H or C₁-C₃ alkyl;
R² is

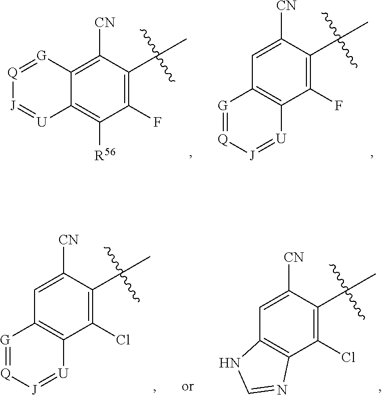

where R⁵⁶ is hydrogen, fluoro, chloro, or methyl,
G, Q, J and U are independently selected from C(H), C(R⁵), and N, provided only one or two of G, Q, J, and U can be N;
   each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl;
R⁶ is hydrogen, halogen, C₁-C₆ alkyl, hydroxy, C₁-C₆ alkoxy, C₁-C₃ alkoxyC₁-C₃ alkyl, C₃-C₆ heterocycloalkyl, —C(O)—C₁-C₃ haloalkyl, or —NR¹⁵(CO)R¹⁶, where R¹⁵ is hydrogen or methyl, and R¹⁶ is C₁-C₃ alkyl; and
R⁷ is C₁-C₃ alkyl or C₁-C₃ haloalkyl.

One aspect of the disclosure provides the method wherein the PRMT5 inhibitor is a compound of the Formula IIIA:

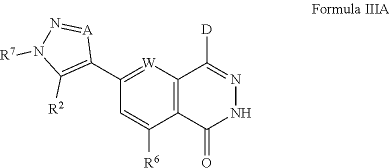

or a pharmaceutically acceptable salt thereof, wherein
A is CR⁹ or N;
D is —CH₂—NH₂,

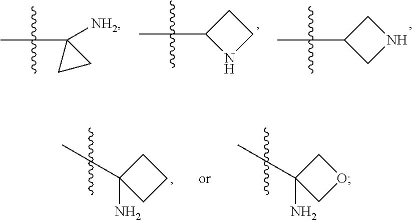 [SMILES: *C1(N)CC1 ; *C1CCN1 ; *C1CNC1 ; *C1(N)CCC1 ; *C1(N)COC1]

W is CR⁹ or N, where R⁹ is H or C₁-C₃ alkyl;
R² is

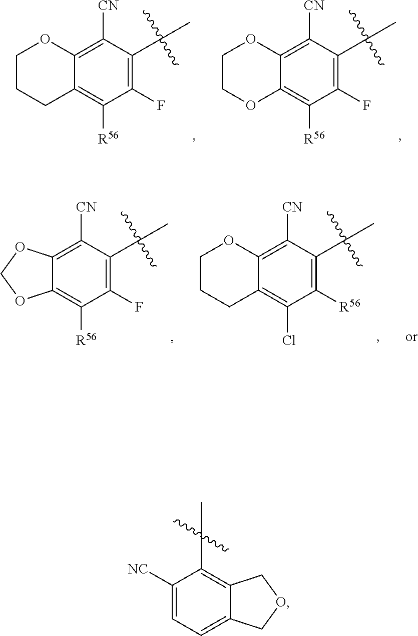

where R⁵⁶ is hydrogen, halogen, C₁-C₆ alkyl, C₁-C₆ haloalkyl, C₁-C₆ alkoxy, or C₁-C₆ haloalkoxy;
R⁶ is hydrogen, halogen, C₁-C₆ alkyl, hydroxy, C₁-C₆ alkoxy, C₁-C₃ alkoxyC₁-C₃ alkyl, C₃-C₆ heterocycloalkyl, —C(O)—C₁-C₃ haloalkyl, or —NR¹⁵(CO)R¹⁶, where R¹⁵ is hydrogen or methyl, and R¹⁶ is C₁-C₃ alkyl; and
R⁷ is C₁-C₃ alkyl or C₁-C₃ haloalkyl.

In certain embodiments of Formula IIIA, A is CH.

In certain embodiments of Formula IIIA, W is N.

In certain embodiments of Formula IIIA, W is CH.

In certain embodiments of Formula IIIA, D is —CH₂—NH₂.

In certain embodiments of Formula IIIA, the PRMT5 inhibitor is of the formula:

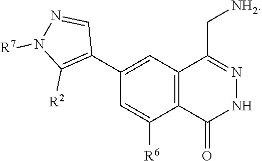

In certain embodiments of Formula IIIA, R² is

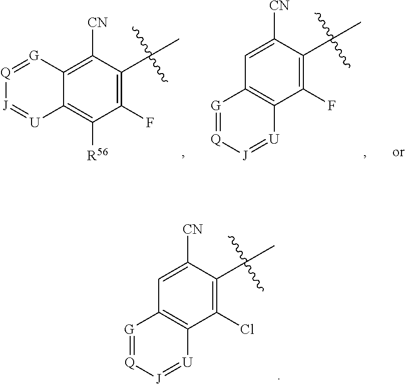

In certain embodiments of Formula IIIA, G, Q, J and U are independently selected from C(H) and C(R⁵).

In certain embodiments of Formula IIIA, G, Q, J and U are independently C(H).

In certain embodiments of Formula IIIA, at least one of G, Q, J, and U is C(R⁵), and the remaining G, Q, J, and U are independently C(H); for example only one of G, Q, J, and U is C(R⁵).

In certain embodiments of Formula IIIA, U is N, and G, Q, and J are independently selected from C(H) and C(R⁵).

In certain embodiments of Formula IIIA, G is N, and Q, J, and U are independently selected from C(H) and C(R⁵).

In certain embodiments of Formula IIIA, R⁵, if present, is hydroxy, halogen, C₁-C₃ alkyl, C₁-C₃ haloalkyl, C₁-C₃ alkoxy, C₁-C₃ haloalkoxy, C₃-C₆ cycloalkoxy, C₃-C₆ cycloalkyl, C₃-C₆ heterocycloalkyl, or C₁-C₃ alkoxyC₁-C₃ alkyl.

In certain embodiments of Formula IIIA, R⁵, if present, is hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl.

In certain embodiments of Formula IIIA, R⁵, if present, is hydroxy, chloro, fluoro, methyl, ethyl, methoxy, ethoxy, 2,2-difluoroethoxy, oxetanyl, tetrahydrofuranyl, (methoxy)methyl, (ethoxy)methyl, (methoxy)ethyl, or (ethoxy)ethyl.

In certain embodiments of Formula IIIA, R⁵, if present, is halogen, C₁-C₆ alkyl, or C₁-C₆ alkoxy; for example, R⁶ is halogen, C₁-C₃ alkyl, or C₁-C₃ alkoxy.

In certain embodiments of Formula IIIA, R⁵, if present, is chloro, fluoro, methyl, ethyl, methoxy, or ethoxy.

In certain embodiments of Formula IIIA, R⁵⁶ is fluoro, chloro, or methyl.

In certain embodiments of Formula IIIA, R² is

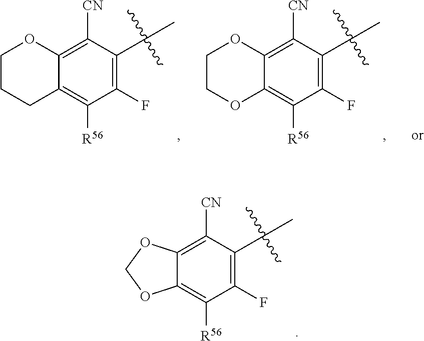

In certain embodiments of Formula IIIA, R⁵⁶ is hydrogen, fluoro, chloro, or methyl.

In certain embodiments of Formula IIIA, R⁶ is hydrogen, halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶; for example, wherein R⁶ is hydrogen, chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)— difluoromethyl, —NH₂, or —NH(CO)CH₃.

In certain embodiments of Formula IIIA, R⁶ is hydrogen, halogen, C₁-C₆ alkyl, or C₁-C₆ alkoxy; for example, R⁶ is hydrogen, halogen, C₁-C₃ alkyl, or C₁-C₃ alkoxy.

In certain embodiments of Formula IIIA, R⁶ is hydrogen, chloro, fluoro, methyl, ethyl, methoxy, or ethoxy.

In certain embodiments of Formula IIIA, R⁶ is halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶; for example, wherein R⁶ is chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

In certain embodiments of Formula IIIA, R⁶ is halogen, C₁-C₆ alkyl, or C₁-C₆ alkoxy; for example, R⁶ is halogen, C₁-C₃ alkyl, or C₁-C₃ alkoxy.

In certain embodiments of Formula IIIA, R⁶ is chloro, fluoro, methyl, ethyl, methoxy, or ethoxy.

In certain embodiments of Formula IIIA, R⁷ is methyl.

In certain embodiments of Formula IIIA, R⁷ is ethyl.

In certain embodiments of Formula IIIA, R⁷ is propyl (e.g., isopropyl).

In certain embodiments of Formula IIIA, R⁷ is difluoromethyl or trifluoromethyl.

In certain embodiments of the methods of the disclosure as described herein, the PRMT5 inhibitor is:

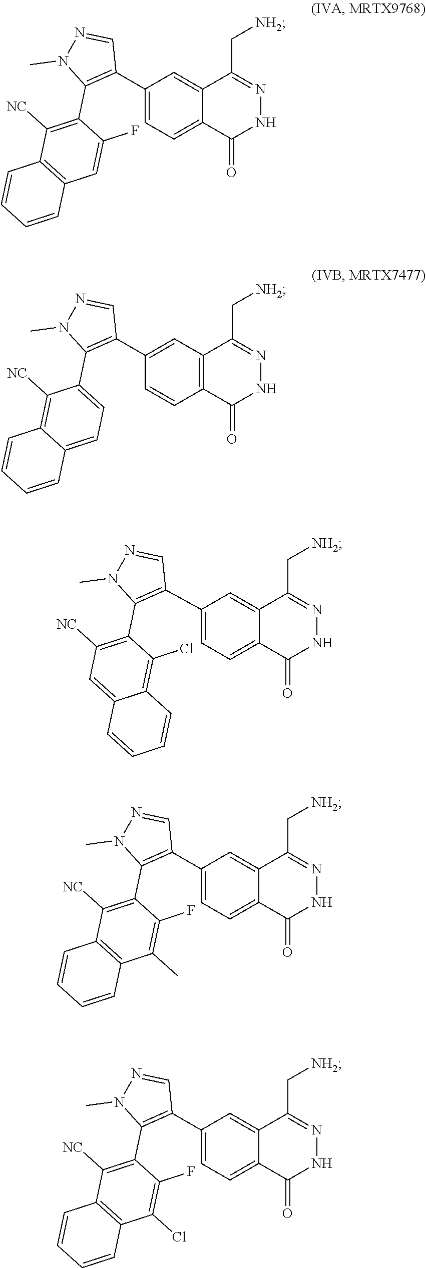 [SMILES: Cn1ncc(-c2ccc3c(=O)[nH]nc(CN)c3c2)c1-c1c(F)cc2ccccc2c1C#N ; Cn1ncc(-c2ccc3c(=O)[nH]nc(CN)c3c2)c1-c1ccc2ccccc2c1C#N ; Cn1ncc(-c2ccc3c(=O)[nH]nc(CN)c3c2)c1-c1c(C#N)cc2ccccc2c1Cl ; Cc1c(F)c(-c2c(-c3ccc4c(=O)[nH]nc(CN)c4c3)cnn2C)c(C#N)c2ccccc12 ; Cn1ncc(-c2ccc3c(=O)[nH]nc(CN)c3c2)c1-c1c(F)c(Cl)c2ccccc2c1C#N]

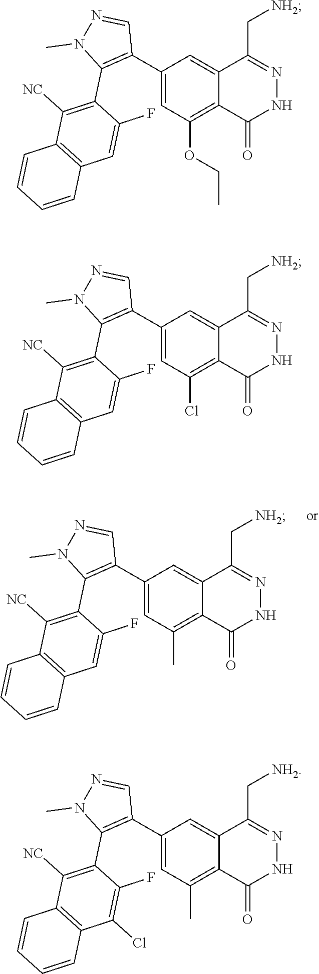 [SMILES: CCOc1cc(-c2cnn(C)c2-c2c(F)cc3ccccc3c2C#N)cc2c(CN)n[nH]c(=O)c12 ; Cn1ncc(-c2cc(Cl)c3c(=O)[nH]nc(CN)c3c2)c1-c1c(F)cc2ccccc2c1C#N ; Cc1cc(-c2cnn(C)c2-c2c(F)cc3ccccc3c2C#N)cc2c(CN)n[nH]c(=O)c12 ; Cc1cc(-c2cnn(C)c2-c2c(F)c(Cl)c3ccccc3c2C#N)cc2c(CN)n[nH]c(=O)c12]

In certain embodiments of the methods of the disclosure as described herein, the PRMT5 inhibitor is:

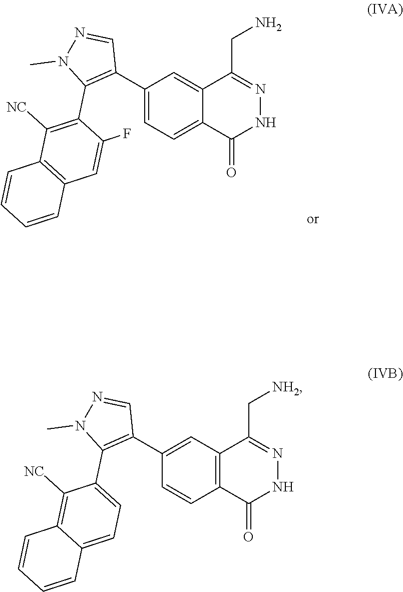 [SMILES: Cn1ncc(-c2ccc3c(=O)[nH]nc(CN)c3c2)c1-c1c(F)cc2ccccc2c1C#N ; Cn1ncc(-c2ccc3c(=O)[nH]nc(CN)c3c2)c1-c1ccc2ccccc2c1C#N]

or a pharmaceutically acceptable salt thereof.

In certain embodiments of the methods of the disclosure as described herein, the PRMT5 inhibitor is:

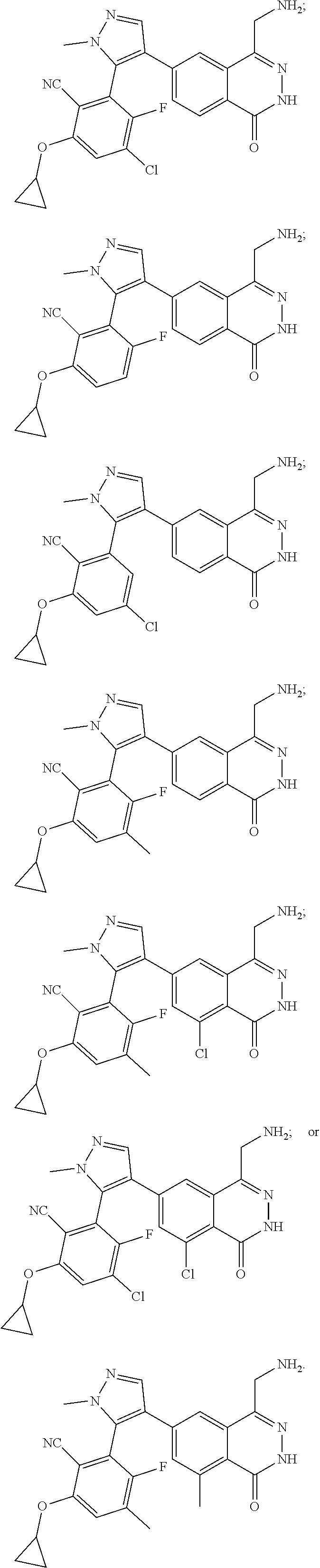

In certain embodiments of the methods of the disclosure as described herein, the PRMT5 inhibitor is:

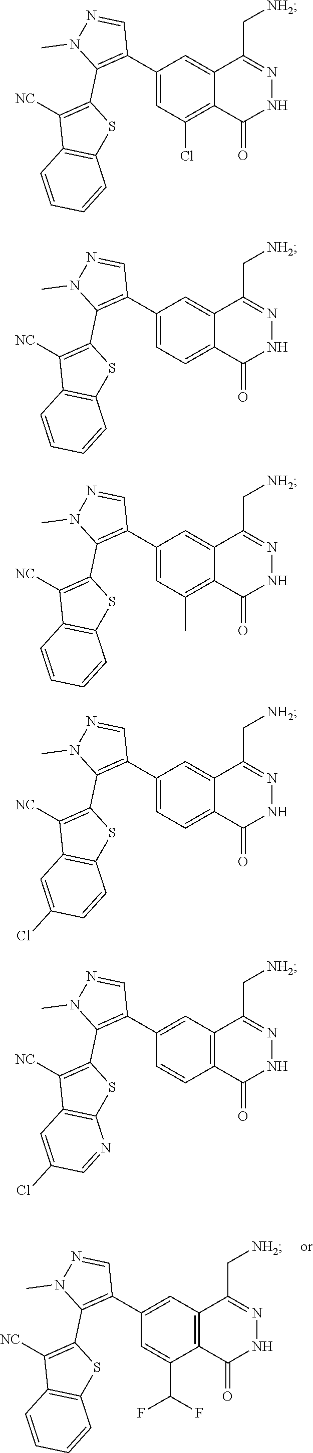

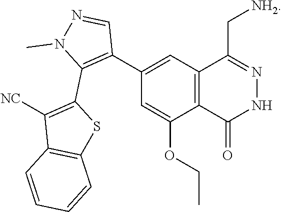 [SMILES: CCOc1cc(-c2cnn(C)c2-c2sc3ccccc3c2C#N)cc2c(CN)n[nH]c(=O)c12]

In certain embodiments of the methods of the disclosure as described herein, the PRMT5 inhibitor is:

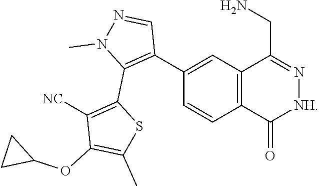 [SMILES: Cc1sc(-c2c(-c3ccc4c(=O)[nH]nc(CN)c4c3)cnn2C)c(C#N)c1OC1CC1]

In certain embodiments as described herein, the PRMT5 inhibitor is

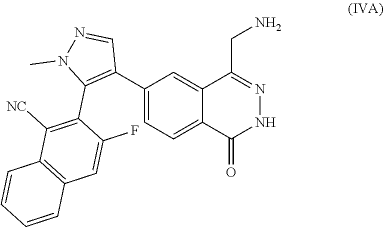 [SMILES: Cn1ncc(-c2ccc3c(=O)[nH]nc(CN)c3c2)c1-c1c(F)cc2ccccc2c1C#N]

or a pharmaceutically acceptable salt, and the SOS1 inhibitor is compound of Formula I or a pharmaceutically acceptable salt thereof. For example, in som embodiments, the PRMT5 inhibitor is

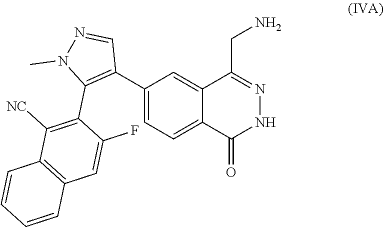 [SMILES: Cn1ncc(-c2ccc3c(=O)[nH]nc(CN)c3c2)c1-c1c(F)cc2ccccc2c1C#N]

or a pharmaceutically acceptable salt, and the SOS1 inhibitor is MRTX-0902 or a pharmaceutically acceptable salt thereof.

In certain embodiments as described herein, the PRMT5 inhibitor is

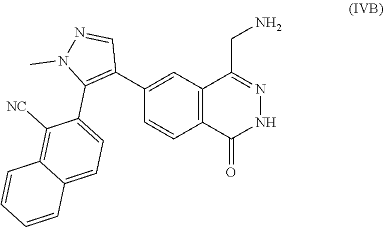 [SMILES: Cn1ncc(-c2ccc3c(=O)[nH]nc(CN)c3c2)c1-c1ccc2ccccc2c1C#N]

or a pharmaceutically acceptable salt, and the SOS1 inhibitor is compound of Formula I or a pharmaceutically acceptable salt thereof. For example, in some embodiments, the PRMT5 inhibitor is

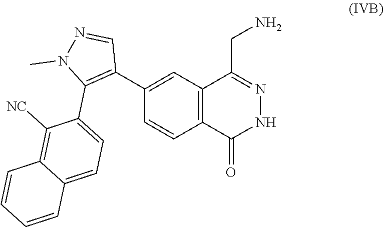 [SMILES: Cn1ncc(-c2ccc3c(=O)[nH]nc(CN)c3c2)c1-c1ccc2ccccc2c1C#N]

or a pharmaceutically acceptable salt, and the SOS1 inhibitor is MRTX-0902 or a pharmaceutically acceptable salt thereof.

In certain embodiments as described herein, the PRMT5 inhibitor is

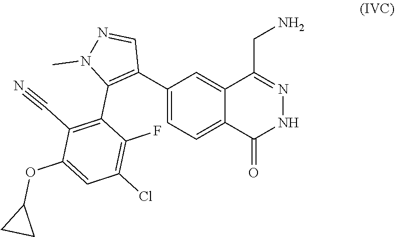 [SMILES: Cn1ncc(-c2ccc3c(=O)[nH]nc(CN)c3c2)c1-c1c(F)c(Cl)cc(OC2CC2)c1C#N]

or a pharmaceutically acceptable salt thereof, and the SOS1 inhibitor is a compound of Formula 1 or a pharmaceutically acceptable salt thereof. For example, in some embodiments, the PRMT5 inhibitor is

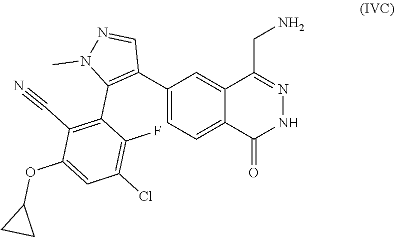 [SMILES: Cn1ncc(-c2ccc3c(=O)[nH]nc(CN)c3c2)c1-c1c(F)c(Cl)cc(OC2CC2)c1C#N]

or a pharmaceutically acceptable salt thereof, and the SOS1 inhibitor is MRTX-0902 or a pharmaceutically acceptable salt thereof.

In some embodiments as described herein, the PRMT5 inhibitor is MRTX1719 or a pharmaceutically acceptable salt thereof, and the SOS1 inhibitor is MRTX0902 or a pharmaceutically acceptable salt thereof.

In certain embodiments as described herein, the PRMT5 inhibitor is:

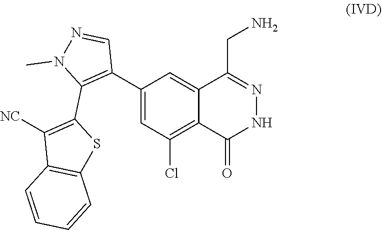 [SMILES: Cn1ncc(-c2cc(Cl)c3c(=O)[nH]nc(CN)c3c2)c1-c1sc2ccccc2c1C#N]

or a pharmaceutically acceptable salt thereof, and the SOS1 inhibitor is a compound of Formula 1 or a pharmaceutically acceptable salt thereof. For example, in some embodiments, the PRMT5 inhibitor is:

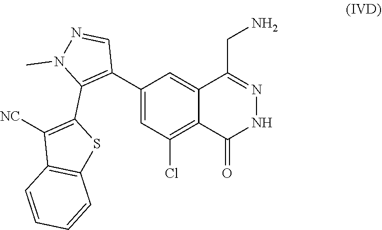 [SMILES: Cn1ncc(-c2cc(Cl)c3c(=O)[nH]nc(CN)c3c2)c1-c1sc2ccccc2c1C#N]

or a pharmaceutically acceptable salt thereof, and the SOS1 inhibitor is MRTX-0902 or a pharmaceutically acceptable salt thereof.

### Definitions

For simplicity, chemical moieties are defined and referred to throughout primarily as univalent chemical moieties (e.g., alkyl, aryl, etc.). Nevertheless, such terms may also be used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for 0, and 2, 4, or 6 for S, depending on the oxidation state of the S).

The term “amino” refers to —NH₂.

The term “acetyl” refers to “—C(O)CH₃.

As herein employed, the term “acyl” refers to an alkylcarbonyl or arylcarbonyl substituent wherein the alkyl and aryl portions are as defined herein.

The term “alkyl” as employed herein refers to saturated straight and branched chain aliphatic groups having from 1 to 12 carbon atoms. As such, “alkyl” encompasses C₁, C₂, C₃, C₄, C₅, C₆, C₇, C₈, C₉, C₁₀, C₁₁ and C₁₂ groups. Examples of alkyl groups include, without limitation, methyl, ethyl, propyl, isopropyl, butyl, isobutyl, sec-butyl, tert-butyl, pentyl, and hexyl.

The term “alkenyl” as used herein means an unsaturated straight or branched chain aliphatic group with one or more carbon-carbon double bonds, having from 2 to 12 carbon atoms. As such, “alkenyl” encompasses C₂, C₃, C₄, C₅, C₆, C₇, C₈, C₉, C₁₀, C₁₁ and C₁₂ groups.

Examples of alkenyl groups include, without limitation, ethenyl, propenyl, butenyl, pentenyl, and hexenyl.

The term “alkynyl” as used herein means an unsaturated straight or branched chain aliphatic group with one or more carbon-carbon triple bonds, having from 2 to 12 carbon atoms. As such, “alkynyl” encompasses C₂, C₃, C₄, C₅, C₆, C₇, C₈, C₉, C₁₀, C₁₁ and C₁₂ groups. Examples of alkynyl groups include, without limitation, ethynyl, propynyl, butynyl, pentynyl, and hexynyl.

An “alkylene,” “alkenylene,” or “alkynylene” group is an alkyl, alkenyl, or alkynyl group, as defined hereinabove, that is positioned between and serves to connect two other chemical groups. Examples of alkylene groups include, without limitation, methylene, ethylene, propylene, and butylene. Exemplary alkenylene groups include, without limitation, ethenylene, propenylene, and butenylene. Exemplary alkynylene groups include, without limitation, ethynylene, propynylene, and butynylene.

The term “alkoxy” refers to —OC₁-C₆ alkyl.

The term “cycloalkyl” as employed herein is a saturated and partially unsaturated cyclic hydrocarbon group having 3 to 12 carbons. As such, “cycloalkyl” includes C₃, C₄, C₅, C₆, C₇, C₈, C₉, C₁₀, C₁₁ and C₁₂ cyclic hydrocarbon groups. Examples of cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” refers to an alkyl group, as defined hereinabove, wherein one or more carbon atoms in the chain are independently replaced O, S, or NR^{x}, wherein R^{x} is hydrogen or C₁-C₃ alkyl. Examples of heteroalkyl groups include methoxymethyl, methoxyethyl and methoxypropyl.

An “aryl” group is a C₆-C₁₄ aromatic moiety comprising one to three aromatic rings. As such, “aryl” includes C₆, C₁₀, C₁₃, and C₁₄ cyclic hydrocarbon groups. An exemplary aryl group is a C₆-C₁₀ aryl group. Particular aryl groups include, without limitation, phenyl, naphthyl, anthracenyl, and fluorenyl. An “aryl” group also includes fused multicyclic (e.g., bicyclic) ring systems in which one or more of the fused rings is non-aromatic, provided that at least one ring is aromatic, such as indenyl.

An “aralkyl” or “arylalkyl” group comprises an aryl group covalently linked to an alkyl group wherein the moiety is linked to another group via the alkyl moiety. An exemplary aralkyl group is —(C₁-C₆)alkyl(C₆-C₁₀)aryl, including, without limitation, benzyl, phenethyl, and naphthylmethyl. For example, an arC1-C₃alkyl is an aryl group covalently linked to a C₁-C₃ alkyl.

A “heterocyclyl” or “heterocyclic” group is a mono- or bicyclic (fused or spiro) ring structure having from 3 to 12 atoms, (3, 4, 5, 6, 7, 8, 9, 10, 11 or 12 atoms), for example 4 to 8 atoms, wherein one or more ring atoms are independently —C(O)—, N, NR⁴, O, or S, and the remainder of the ring atoms are quaternary or carbonyl carbons. Examples of heterocyclic groups include, without limitation, epoxy, oxiranyl, oxetanyl, azetidinyl, aziridinyl, tetrahydrofuranyl, tetrahydropyranyl, tetrahydrothiophenyl, pyrrolidinyl, piperidinyl, piperazinyl, imidazolidinyl, thiazolidinyl, thiatanyl, dithianyl, trithianyl, azathianyl, oxathianyl, dioxolanyl, oxazolidinyl, oxazolidinonyl, decahydroquinolinyl, piperidonyl, 4-piperidonyl, thiomorpholinyl, dimethyl-morpholinyl, and morpholinyl. Specifically excluded from the scope of this term are compounds having adjacent ring O and/or S atoms.

As used herein, “L-heterocyclyl” refers to a heterocyclyl group covalently linked to another group via an alkylene linker.

As used herein, the term “heteroaryl” refers to a group having 5 to 14 ring atoms, preferably 5, 6, 10, 13 or 14 ring atoms; having 6, 10, or 14 π electrons shared in a cyclic array; and having, in addition to carbon atoms, from one to three heteroatoms that are each independently N, O, or S. Heteroaryl also includes fused multicyclic (e.g., bicyclic) ring systems in which one or more of the fused rings is non-aromatic, provided that at least one ring is aromatic and at least one ring contains an N, O, or S ring atom. Examples of heteroaryl groups include acridinyl, azocinyl, benzimidazolyl, benzofuranyl, benzo[d]oxazol-2(3H)-one, 2H-benzo[b][1,4]oxazin-3(4H)-one, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

A “L-heteroaralkyl” or “L-heteroarylalkyl” group comprises a heteroaryl group covalently linked to another group via an alkylene linker. Examples of heteroalkyl groups comprise a C₁-C₆ alkyl group and a heteroaryl group having 5, 6, 9, or 10 ring atoms. Examples of heteroaralkyl groups include pyridylmethyl, pyridylethyl, pyrrolylmethyl, pyrrolylethyl, imidazolylmethyl, imidazolylethyl, thiazolylmethyl, thiazolylethyl, benzimidazolylmethyl, benzimidazolylethyl quinazolinylmethyl, quinolinylmethyl, quinolinylethyl, benzofuranylmethyl, indolinylethyl isoquinolinylmethyl, isoinodylmethyl, cinnolinylmethyl, and benzothiophenylethyl. Specifically excluded from the scope of this term are compounds having adjacent ring O and/or S atoms.

An “arylene,” “heteroarylene,” or “heterocyclylene” group is a bivalent aryl, heteroaryl, or heterocyclyl group, respectively, as defined hereinabove, that is positioned between and serves to connect two other chemical groups.

As employed herein, when a moiety (e.g., cycloalkyl, aryl, heteroaryl, heterocyclyl, urea, etc.) is described as “optionally substituted” without expressly stating the substituents it is meant that the group optionally has from one to four, preferably from one to three, more preferably one or two, non-hydrogen substituents.

The term “halogen” or “halo” as employed herein refers to chlorine, bromine, fluorine, or iodine.

The term “haloalkyl” refers to an alkyl chain in which one or more hydrogens have been replaced by a halogen. Exemplary haloalkyls are trifluoromethyl, difluoromethyl, flurochloromethyl, chloromethyl, and fluoromethyl.

The term “hydroxyalkyl” refers to -alkylene-OH.

### EXAMPLE

The methods of the disclosure are illustrated further by the following examples, which is not to be construed as limiting the disclosure in scope or spirit to the specific procedures and compounds described in them.

### MRTX1719+SOS1 Combination Study Procedure:

Immunodeficient female mice were implanted with human cancer models with homozygous deletion of the MTAP gene (MTAP^{DEL}). Mouse health was monitored daily, and caliper measurements began when tumors were palpable. Tumor volume measurements were determined utilizing the formula 0.5×L×W2 in which L refers to length and W refers to width of each tumor. When tumors reached approximately 100-175 mm³. Animals were randomized to receive A) vehicle (0.5% methylcellulose (4000 cps)/0.2% Tween80 in water), B) a PRMT5 inhibitor, C) SOS1 inhibitor, or D) the PRMT5 inhibitor and SOS1 inhibitor, all administered orally (PO). Tumor volumes were measured twice a week (n=4 or 5/treatment group). Tumor Growth Inhibition (% TGI) was calculated when the average final treated tumor volume was greater than initial treated tumor volume using the formula: (1−(Final Drug Treated Tumor Volume—Initial Drug Treated Tumor Volume)/(Final Vehicle Treated Tumor Volume—Initial Vehicle Treated Tumor Volume))*100. Percent Regression (% Regression) was calculated when the average tumor volume of final treated tumors was less than initial treated tumor volume using the formula: (-100%)*(1−((Final treated tumor volume)/(Initial treated tumor volume)).

### Example 1

This example was conducted in immunodeficient female NMRI-Foxnlnu mice that were implanted with a human Malignant Peripheral Nerve Sheath Tumor (MPNST) patient derived xenograft (PDX) model according to the study procedure described above. The PRMT5 inhibitor was MRTX1719 administered at 100 mg/kg once a day (QD). MRTX1719 used is as described herein.

The SOS1 inhibitor used in this example was MRTX0902 administered at 50 mg/kg twice a day (BID). MRTX0902 used is as described herein.

Results are provided in Table 1. The combination of MRTX1719 and MRTX0902 led to greater antitumor activity compared to either inhibitor alone in the MPNST MTAP^{DEL} and NF1 mutant MPNST human PDX model.

TABLE 1

| | Monotherapy Treatment Groups | | | |
|---|---|---|---|---|
| | | MRTX1719 | MRTX0902 | Combination |
| | | 100 mg/kg | 50 mg/kg | MRTX1719 + |
| Study Day | Vehicle | QD | BID | MRTX0902 |
| 0 | 124 | 109 | 115 | 115 |
| 3 | 151 | 128 | 143 | 120 |
| 7 | 175 | 146 | 173 | 119 |
| 10 | 196 | 124 | 185 | 109 |
| 14 | 210 | 113 | 157 | 91 |
| 17 | 251 | 138 | 211 | 104 |
| 21 | 236 | 112 | 168 | 82 |
| 24 | 244 | 116 | 158 | 72 |
| 28 | 237 | 116 | 170 | 66 |
| 31 | 264 | 121 | 178 | 66 |
| 35 | 291 | 122 | 199 | 61 |
| % TGI | — | 92.3 | 49.9 | — |
| % Regression | — | — | — | −46.8 |

### Example 2

This example was conducted in immunodeficient female nu/nu mice that were implanted with 5×10⁶ MIA-Paca-2 pancreatic cancer cells in 50% Matrigel. according to the study procedure described above. The PRMT5 inhibitor was MRTX1719 administered at 100 mg/kg once a day (QD). MRTX1719 used is as described herein.

The SOS11 inhibitor used in this example was MRTX0902 administered at 50 mg/kg twice a day (BID). MRTX0912 used is as described herein.

Results are provided in Table 2. The combination of MRTX1719 and MRTX0902 led to greater antitumor activity compared to either inhibitor alone in the MIA-PaCa-2 MTAP^{DEL} and KRAS^{G12C} pancreatic human tumor xenograft model.

TABLE 2

| | Monotherapy Treatment Groups | | | Combination |
|---|---|---|---|---|
| | | MRTX1719 | MRTX0902 | MRTX1719 + |
| Study Day | Vehicle | 100 mg/kg QD | 50 mg/kg BID | MRTX0902 |
| −1 | 162 | 165 | 163 | 169 |
| 7 | 374 | 442 | 314 | 199 |
| 10 | 530 | 533 | 425 | 194 |
| 13 | 590 | 571 | 467 | 235 |
| 16 | 653 | 675 | 530 | 259 |
| 20 | 742 | 745 | 567 | 275 |
| % TGI | — | 0.0 | 30.2 | 81.6 |

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

1. A method for treating cancer in a subject, the method comprising:
administering to the subject a therapeutically effective amount of a SOS1 inhibitor and a therapeutically effective amount of a methylthioadenosine (MTA)-cooperative protein arginine N-methyl transferase 5 (PRMT5) inhibitor.

2. The method of claim 1, wherein the cancer comprises methylthioadenosine phosphorylase (MTAP) gene homozygous deletion.

3. The method of claim 1, wherein the cancer comprises NF1 gene mutation.

4. The method of claim 1, wherein the cancer is a malignant peripheral nerve sheath tumors (MPNST).

5. The method of claim 1, wherein the SOS1 inhibitor is selected from BI 1701963, BTX-B01, RGT-018, HM99462, RMC-5845, and combinations thereof.

6. The method of claim 1, wherein the SOS1 inhibitor is 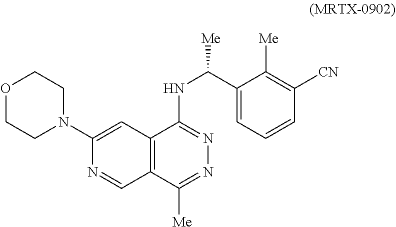 [SMILES: Cc1c(C#N)cccc1[C@@H](C)Nc1nnc(C)c2cnc(N3CCOCC3)cc12]
or a pharmaceutical salt thereof.

7. The method of claim 16, wherein the PRMT5 inhibitor is: 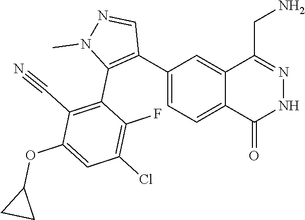 [SMILES: Cn1ncc(-c2ccc3c(=O)[nH]nc(CN)c3c2)c1-c1c(F)c(Cl)cc(OC2CC2)c1C#N]
or a pharmaceutically acceptable salt thereof.

8. The method of claim 16, wherein the PRMT5 inhibitor is: 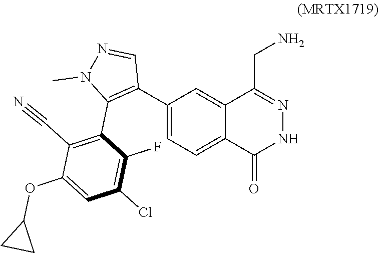
or a pharmaceutically acceptable salt thereof.

9. The method of claim 1, wherein the PRMT5 inhibitor is 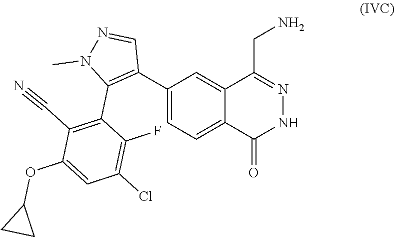 [SMILES: Cn1ncc(-c2ccc3c(=O)[nH]nc(CN)c3c2)c1-c1c(F)c(Cl)cc(OC2CC2)c1C#N]
or a pharmaceutically acceptable salt thereof, and the SOS1 inhibitor is a compound of Formula 1 or a pharmaceutically acceptable salt thereof.

10. The method of claim 1, wherein the PRMT5 inhibitor is 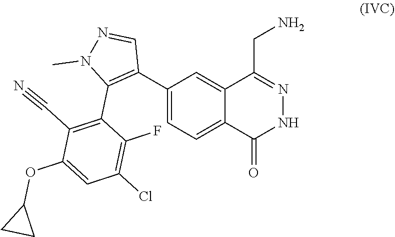 [SMILES: Cn1ncc(-c2ccc3c(=O)[nH]nc(CN)c3c2)c1-c1c(F)c(Cl)cc(OC2CC2)c1C#N]
or a pharmaceutically acceptable salt thereof, and the SOS1 inhibitor is MRTX-0902 or a pharmaceutically acceptable salt thereof.

11. The method of claim 1, wherein the PRMT5 inhibitor is MRTX1719 or a pharmaceutically acceptable salt thereof, and the SOS1 inhibitor is MRTX0902 or a pharmaceutically acceptable salt thereof.

12. The method of claim 1, wherein the therapeutically effective amount of the PRMT5 inhibitor is in the range of about 0.01 to 300 mg/kg per day.

13. The method of claim 1, wherein the therapeutically effective amount of the PRMT5 inhibitor is in the range of about 0.1 to 100 mg/kg per day.

14. The method of claim 1, wherein the therapeutically effective amount of the PRMT5 inhibitor is less than 1% of, e.g., less than 10%, or less than 25%, or less than 50% of the clinically-established therapeutic amount.

15. The method of claim 12, wherein the therapeutically effective amount of the PRMT5 inhibitor is administered once daily.

16. The method of claim 1, wherein the therapeutically effective amount of the SOS1 inhibitor is in the range of about 0.01 to 300 mg/kg per day.

17. The method of claim 1, wherein the therapeutically effective amount of the SOS1 inhibitor is in the range of about 0.1 to 100 mg/kg per day.

18. The method of claim 1, wherein the therapeutically effective amount of the SOS1 inhibitor is less than 1% of, e.g., less than 10%, or less than 25%, or less than 50% of the clinically-established therapeutic amount.

19. The method of claim 16, wherein the therapeutically effective amount of the PRMT5 inhibitor is administered twice daily.

20. The method of claim 1, wherein the SOS1 inhibitor and the PRMT5 inhibitor are administered sequentially.

21. The method of claim 1, wherein the SOS1 inhibitor and the PRMT5 inhibitor are administered simultaneously.

22. The method of claim 1, wherein the subject previously received or completed a first-line chemotherapy.

23. The method of claim 1, wherein the subject did not previously received or complete a first-line chemotherapy.

24. The method of claim 22, wherein the first-line chemotherapy is platinum- and/or taxane-based chemotherapy.
